(19) United States
(12) Patent Application Publication
(10) Pub. No.: US 2024/0216476 A1
(43) Pub. Date: Jul. 4, 2024

(54) COMBINATION OF BITTER RECEPTOR AGONIST AND GUT-SIGNALING COMPOUND

(71) Applicant: AARDVARK THERAPEUTICS, INC., San Diego, CA (US)

(72) Inventors: Zhenhuan ZHENG, San Diego, CA (US); Andreas NIETHAMMER, San Diego, CA (US); Anjuli TIMMER, San Diego, CA (US); Tien-Li LEE, San Diego, CA (US)

(73) Assignee: AARDVARK THERAPEUTICS, INC., San Diego, CA (US)

(21) Appl. No.: 18/557,182

(22) PCT Filed: Apr. 26, 2022
(86) PCT No.: PCT/US2022/026381

§ 371 Date: Oct. 25, 2023

Related U.S. Application Data
(60) Provisional application No. 63/180,224, filed on Apr. 27, 2021.
(60) Provisional application No. 63/229,499, filed on Aug. 4, 2021.
(60) Provisional application No. 63/245,925, filed on Sep. 19, 2021.
(60) Provisional application No. 63/305,037, filed on Jan. 31, 2022.

(51) Int. Cl. A61K 38/26; A61K 9/20; A61K 9/48; A61K 31/167; A61K 31/4985; A61K 45/06; A61P 3/04

(52) U.S. Cl. CPC A61K 38/26; A61K 9/2086; A61K 9/4825; A61K 31/167; A61K 31/4985; A61K 45/06; A61P 3/04

(57) ABSTRACT

There is disclosed a combination oral dosage form pharmaceutical composition comprising a bitter receptor agonist and a gut-signaling compound. i.e., a gut-signaling peptide analog and/or gut-signaling hormone enhancer. And there is disclosed a method for treating obesity, diabetes, metabolic syndrome, glycemic control hyperlipidemia, and effecting weight loss comprising administering an effective amount of a pharmaceutical composition comprising a bitter receptor agonist and a gut-signaling compound, i.e., a gut-signaling peptide analog and/or gut-signaling hormone enhancer, as described above and herein. There is further disclosed a method for preventing progression and/or treating a fatty liver disease, comprising administering an effective amount of a combination comprising a bitter receptor agonist comprising a denatonium salt, wherein the denatonium salt is selected from the group consisting of denatonium acetate (DA), denatonium citrate, denatonium maleate, denatonium saccharide, and denatonium tartrate: and a GLP-1 receptor agonist.

### CROSS-REFERENCE TO RELATED APPLICATIONS

This application claims the benefit of priority of U.S. Provisional Application No. 63/180,224, filed Apr. 27, 2021; U.S. Provisional Application No. 63/229,499, filed Aug. 4, 2021; U.S. Provisional Application No. 63/245,925, filed Sep. 19, 2021; and U.S. Provisional Application No. 63/305,037, filed Jan. 31, 2022, each of which is incorporated by reference herein in its entirety for any purpose.

### TECHNICAL FIELD

The present disclosure provides a combination of a bitter receptor agonist (alternatively referred to as a TAS2R or T2R agonist), and at least one gut-signaling compound selected from a gut-signaling peptide analog and/or gut-signaling hormone enhancer. Also provided are therapeutic uses of such a combination, e.g., for treating glucagon-related diseases, disorders, and conditions, as defined herein, including, for example, diabetes, prediabetes syndrome, obesity, weight and/or appetite control, hyperlipidemia, and hyperglycemia. The present disclosure further provides, inter alia, a method for preventing progression of, or treating, a fatty liver disease, comprising administering a combination comprising a bitter receptor agonist and a GLP-1 receptor agonist.

### INTRODUCTION

Over the past 40 years, global levels of obesity have more than doubled. Obesity predisposes to metabolic syndrome and has been linked to coronary heart disease, stroke, type 2 diabetes, and certain forms of cancer. Obesity has even been linked to greater risk of severe illness and higher risk of death due to the coronavirus, one of the most significant current global health challenges. In tandem with the emergence of this problem has been an increase in understanding the pathological mechanisms which link an obese state to the development of disease. Central to these mechanisms is the heightened state of systemic inflammation arising from obesity, resulting in a multitude of pathologies. Therefore, there is a significant need for treatments and preventives to address appetite and inflammatory signals and optimize metabolism. The present disclosure addresses this need and provides other benefits.

According to the HIS Division of Diabetes Treatment and Prevention (last updated April 2021), when a patient has metabolic syndrome and is overweight, that individual has a higher risk for developing type 2 diabetes. Generally, the initial treatment algorithm for treating such a patient (measured by fasting glucose levels and HbA1c for glycemic control) is to treat with oral agents before transitioning to injectable drugs which may be implemented as signs and symptoms indicate insufficient glycemic control.

For example, an overweight patient who presents as diabetic or pre-diabetic typically may be treated by (1) starting with generic metformin; (2) if not sufficiently treated then add an oral DPP-4 inhibitor or use a combination of metformin/DPP-4 inhibitor oral formulation; (3) if still not sufficiently treated, increase the doses of metformin and DPP-4 inhibitor; and lastly (4) failing sufficient treatment, switch to injectable insulin. There are also branded GLP-1 analogs that are primarily injectable with label claims to lower HbA1c and current clinical studies for weight loss and GIP analogs. Both GLP-1 and GIP analogs, alone and in combination, are dual incretin peptide mimetic compounds that agonize receptors for both human GIP and GLP-1. But most GLP-1 and GIP analogs are injectable except Novo Nordisk's Rybelsus® (semaglutide), which is an oral GLP-1 analog. However, as published in Wadden et al. (JAMA 2021:325(14): 1403-1413 published online 24 Feb. 2021) and Aroda et al., “PIONEER 1: Randomized Clinical Trial of the Efficacy and Safety of Oral Semaglutide Monotherapy in Comparison with Placebo in Patients with Type 2 Diabetes” Diabetes Care 43:1724-1732, 2019) it appears that efficacy drops significantly for oral semaglutide versus an injected version of the same GLP-1 analog.

### Type 2 Diabetes and Weight Loss

DPP-4 inhibitors (DPP-4i) inhibit dipeptidyl peptidase-4 (DPP-4), thus leading to increased endogenous incretin levels (including GLP-1 and GIP). They represent a class of effective oral therapeutics for the treatment of diabetes, with sitagliptin (Januvia®) being a representative agent of this class. However, despite their utility, DPP-4i's have been found to be limited in their ability to address obesity. There have been observations of relatively minor weight reductions in obese patients taking DPP-4i drugs, but those effects are limited and often transient, presumably due to increased tolerance to the DPP-4i drugs over prolonged and repeated exposure.

In an 18-week trial of in 800 patients with inadequately controlled type-2 diabetes mellitus (T2DM) on metformin, saxagliptin 5 mg daily vs. sitagliptin 100 mg showed similar reductions in hemoglobin A1c (HbA1c) (−0.52 vs. −0.26%) (Scheen et al., “Efficacy and safety of saxagliptin in combination with metformin compared with sitagliptin in combination with metformin in adult patients with type 2 diabetes mellitus.” Diabetes Metab. Res. Rev. (2010) 26:540-9. doi: 10.1002/dmrr.1114)). The risk of hypoglycemia with DPP-4 inhibitors is low given their GLP-1 mediated glucose dependent mechanism of action.

GLP-1 receptor agonists (GLP-1 RAs) are peptide derivatives of either exendin-4 or human GLP-1 designed to resist the activity of DPP-4 and, therefore, have a prolonged half-life. In clinical trials, GLP-1 RAs demonstrated efficacy, improved weight loss and a low risk of hypoglycemia. However, GI adverse events, particularly nausea, vomiting, and diarrhea are seen, as well as severe Black Box warnings of thyroid cancer.

Several clinical trials have directly compared the efficacy and safety of DPP-4 inhibitors and GLP-1 RAs. These studies have generally demonstrated that the GLP-1 RAs provided superior glycemic control and weight loss relative to the DPP-4 inhibitors. Both treatments were associated with a low and comparable incidence of hypoglycemia, but treatment with GLP-1 RAs were associated with a higher incidence of adverse events. According to current clinical guidelines, GLP-1RAs and DPP-4 inhibitors are both indicated for the glycemic management of patients with T2DM across the spectrum of disease. GLP-1RA may be preferred over DPP-4 inhibitors for many patients because of the greater reductions in hemoglobin A1c and weight loss observed in the clinical trials. Therefore, given better side effect profiles, there is a need for a better combination with DPP-4 inhibitors for weight loss without severe side effects of GLP-1 agonists.

Therefore, there is a need in the art for delaying progression towards the requirement of treating type 2 diabetes with insulin or to slow down or prevent progression to a status that requires insulin treatment. This need may be addressed with an invention described herein which can provide surprisingly beneficial effects to either (1) increase the maximum effect (e.g., in some embodiments, weight loss to 10-15% of body weight) of GLP-1 RAs and/or GIP analogs with a combination orally active agent to enhance treatment efficacy for glycemic control or to delay progression to insulin, and/or (2) improve effectiveness of DPP-4 inhibitors to be at least equivalent or superior to GLP-1 analogs (or combination of GLP-1 analogs with GIP analogs) to provide oral dosing alternatives to injectables.

### Obesity and Weight Loss

Obesity, which is defined in general terms as an excess of body fat relative to lean body mass, is now a world-wide epidemic, and is one of the most serious contributors to increased morbidity and mortality. Obesity is prevalent in the United States, affecting more than 61% of the total population (Flegal et al., Int. J. Obes. 22:39-47, 1998). Obesity is defined more specifically by the United States Centers for Disease Control and Prevention (CDC) as an excessively high amount of body fat or adipose tissue in relation to lean body mass and overweight is defined as an increased body weight in relation to height, when compared to some standard of acceptable or desirable weight. The CDC alternatively defines overweight as a person with a body mass index (BMI) between 25.0 and 29.9 and obesity is defined as a BMI greater than or equal to 30.0. Obesity is often associated with psychological and medical morbidities, the latter of which includes increased joint problems, vascular diseases such as coronary artery disease, hypertension, stroke, and peripheral vascular disease. Obesity also causes metabolic abnormalities such as insulin resistance and Type II diabetes (non-insulin-dependent diabetes mellitus (NIDDM)), hyperlipidemia, and endothelial dysfunction. These abnormalities predispose the vasculature to injury, cellular proliferation and lipid oxidation, with resulting atherosclerosis leading to heart attack, stroke, and peripheral vascular diseases. In 1998, consumers spent $33 billion in the United States for weight-loss products and services with a less than positive outcome (Serdula et al., JAMA 282: 1353-1358, 1999). Thus, obesity and its associated complications continue to be a major problem throughout the worldwide health care system.

Obesity is an important clinical problem with broad reaching implications. Approaches have been limited to diet and exercise (therapeutic lifestyle changes), surgical procedures such as gastric bypass, and pharmacologic agents, including GLP-1 receptor agonists. Drug treatment for obesity has been disappointing since almost all drug treatments for obesity are associated with undesirable side effects that contributed to their termination and/or present unacceptable risk/benefit profiles that either result in termination of treatment and/or offer very limited chance for success. A number of monoamines and neuropeptides reduce food intake (Bray et al., Am. J. Clin. Nutr 0.55:151S-319S, 1992). Available pharmacotherapies have included Sibutramine (an appetite suppressant), Orlistat (a lipase inhibitor), and sympathomimetic drugs fenfluramine and dexfenfluramine. Although body weight loss is effective, the sympathomimetic drugs cause side effects including pulmonary hypertension, neuroanatomic changes, and atypical valvular heart diseases. For example, fenfluramine and dexfenfluramine were withdrawn from the market in 1997 because of associated cardiac valvopathy. Thus, nutrition and dietary restriction are most desirable for weight loss. However, long-term success of dietary regulation is low because of noncompliance.

Thus, there are no ideal treatments based on the biology of the primary metabolic abnormalities found in obesity and its related conditions, such as metabolic syndrome or atherosclerosis. Accordingly, there is still a need for new compositions and methods that address treating individuals suffering from obesity and obesity-related disorders.

### Hyperlipidemia

Hypercholesterolemia is a well-known risk factor for atherosclerotic cardiovascular disease (ASCVD), the major cause of mortality in the Western world. Epidemiological studies have demonstrated that pharmacological lowering of total cholesterol (TC) and Low-density Lipoprotein (LDL) Cholesterol (LDL-C) are associated with a reduction in clinical cardiovascular events.

Triglycerides (TGs) are common types of fats (lipids) that are essential for good health when present in normal amounts. Higher-than-normal triglyceride levels are often associated with known risk factors for heart disease, such as obesity, low levels of high-density lipoproteins (HDLs) (“good”) cholesterol, and high levels of low-density lipoproteins (LDLs) (“bad”) cholesterol. Triglycerides may also contribute to thickening of artery walls; a physical change believed to be a predictor of atherosclerosis. Therefore, high triglyceride levels are at least a warning sign that a patient's heart health may be at risk.

A number of treatments are currently available for lowering serum cholesterol and triglycerides. However, each has its own drawbacks and limitations in terms of efficacy, side-effects and qualifying patient population. Bile-acid-binding resins are a class of drugs that interrupt the recycling of bile acids from the intestine to the liver, e.g., cholestyramine (Questran Light®, Bristol-Myers Squibb), and colestipol hydrochloride (Colestid®, The Upjohn Company). The use of such resins, however, at best only lowers serum cholesterol levels by about 20%, and is associated with gastrointestinal side-effects, including constipation and certain vitamin deficiencies. Moreover, since the resins bind other drugs, other oral medications must be taken at least one hour before or four to six hours subsequent to ingestion of the resin; thus, complicating heart patient's drug regimens.

The statins are cholesterol-lowering agents that block cholesterol synthesis by inhibiting HMGCoA reductase, the key enzyme involved in the cholesterol biosynthetic pathway. The statins, e.g., lovastatin (Mevacor®, Merck & Co., Inc.), simvastatin (Zocor®, Merck & Co., Inc.), atorvastatin (Lipitor®, Pfizer), rosuvastatin (Crestor®, Astra Zeneca) and pravastatin (Pravachol®, Bristol-Myers Squibb Co.), and combinations thereof are sometimes used in combination with bile-acid-binding resins. Statins significantly reduce serum cholesterol and LDL-serum levels, and slow progression of coronary atherosclerosis. However, serum HDL cholesterol levels are only moderately increased. Side effects, including liver and kidney dysfunction are associated with the use of these drugs (Physician's Desk Reference, Medical Economics Co., Inc., Montvale, N.J., 2004; hereinafter “PDR”). The FDA has approved atorvastatin to treat rare but urgent cases of familial hypercholesterolemia.

Ezetimibe is a cholesterol absorption inhibitor which reduces the amount of cholesterol absorbed by the body. Ezetimibe is used to reduce the amount of total cholesterol, LDL cholesterol (by about 18%), and apolipoprotein B. Ezetimibe is often used with a low cholesterol diet and, in some cases, other cholesterol lowering medications.

Niacin, or nicotinic acid, is a water-soluble vitamin B-complex used as a dietary supplement and antihyperlipidemic agent. Niacin diminishes production of VLDL and is effective at lowering LDL. In some cases, it is used in combination with bile-acid binding resins. NIASPAN® has been approved to prevent recurrent heart attacks in patients with high cholesterol. Niacin can increase HDL when used at adequate doses, however, its usefulness is limited by serious side effects when used at such high doses.

Fibric acid derivatives (“fibrates”) are a class of lipid-lowering drugs used to treat various forms of hyperlipidemia (i.e., elevated serum triglycerides) which may also be associated with hypercholesterolemia. Fibrates appear to reduce the VLDL fraction and modestly increase HDL. However, the effects of these drugs on serum cholesterol are variable. Fibrates are mainly used to lower high triglyceride levels. In the United States, fibrates have been approved for use as antilipidemic drugs, but have not received approval as hypercholesterolemia agents.

### Fatty Liver Disease

Fatty liver disease is a term to describe a group of liver diseases including nonalcoholic steatohepatitis (NASH), alcoholic steatohepatitis (ASH), non-alcoholic fatty liver disease (NAFLD), and HIV-associated steatohepatitis, with or without liver fibrosis. NASH is a common liver disease that is associated with increased morbidity and mortality. But there are no FDA-approved treatment options despite many compounds being tested in what are purported to be NASH treatment models. NAFLD is a disorder affecting as many as 1 in 3-5 adults and 1 in 10 children in the United States. These are conditions where there is an accumulation of excess fat in the liver of people who drink little or no alcohol.

The most common form of NAFLD is a non-serious condition called hepatic steatosis (fatty liver), in which fat accumulates in the liver cells: although this is not normal, by itself it probably does not damage the liver. NAFLD most often presents itself in individuals with a constellation of risk factors called the metabolic syndrome, which is characterized by elevated fasting plasma glucose (FPG) with or without intolerance to post-prandial glucose, being overweight or obese, high blood lipids such as cholesterol and triglycerides (TGs) and low high-density lipoprotein cholesterol (HDL-C) levels, and high blood pressure; but not all patients have all the manifestations of the metabolic syndrome. Obesity is thought to be the most common cause of NAFLD; and some experts estimate that about two-thirds of obese adults and one-half of obese children may have fatty liver. The majority of individuals with NAFLD have no symptoms and a normal physical examination (although the liver may be slightly enlarged); children may exhibit symptoms such as abdominal pain and fatigue and may show patchy dark skin discoloration (acanthosis nigricans). The diagnosis of NAFLD is usually first suspected in an overweight or obese person who is found to have mild elevations in their liver blood tests during routine testing, though NAFLD can be present with normal liver blood tests, or incidentally detected on imaging investigations such as abdominal ultrasound or CT scan. It is confirmed by imaging studies, most commonly a liver ultrasound or magnetic resonance imaging (MRI), and exclusion of other causes.

Some people with NAFLD may develop NASH, a more serious condition: about 2-5% of adult Americans and up to 20% of those who are obese may suffer from NASH. In NASH, fat accumulation in the liver is associated with inflammation and different degrees of scarring. NASH is a potentially serious condition that carries a substantial risk of progression to end-stage liver disease, cirrhosis and hepatocellular carcinoma. Some patients who develop cirrhosis are at risk of liver failure and may eventually require a liver transplant. Therefore, weight loss is a recommended means to prevent NASH or slow the progression of NASH. However, weight loss has not been shown to treat NASH once the liver fibrosis damage has occurred.

NAFLD may be differentiated from NASH by the NAFLD Activity Score (NAS), the sum of the histopathology scores of a liver biopsy for steatosis (0 to 3), lobular inflammation (0 to 2), and hepatocellular ballooning (0 to 2). A NAS of <3 corresponds to NAFLD, 3-4 corresponds to borderline NASH, and ≥5 corresponds to NASH. The biopsy is also scored for fibrosis (0 to 4).

NASH is a leading cause of end-stage liver disease.

### Treatments for NAFLD and NASH

There are no drugs currently approved in the US to prevent or treat NAFLD or NASH. A number of pharmacological interventions have been tried in NAFLD/NASH but with overall limited benefit. Antioxidant agents may arrest lipid peroxidation and cytoprotective agents stabilize phospholipid membranes, but agents tried unsuccessfully or with only modest benefit so far include ursodeoxycholic acid, vitamins E (α-tocopherol) and C, and pentoxifylline. Weight-loss agents such as orlistat, have had no significant benefit compared to just the use of diet and exercise to achieve weight loss (“weight loss alone”).

Many weight-loss studies in NAFLD/NASH have been pilot studies of short duration and limited success, reporting only a modest improvement in necroinflammation or fibrosis. A randomized, double-blind, placebo-controlled 6-month trial (Belfort, “A placebo-controlled trial of pioglitazone in subjects with nonalcoholic steatohepatitis”, N. Engl. J. Med., 355, 2297-2307 (2006)) of weight loss alone against pioglitazone, a thiazolidinedione peroxisome proliferator-activated receptor-γ (PPARγ) agonist and insulin sensitizer, failed to demonstrate any improvement for weight loss alone, but treatment with pioglitazone improved glycemic control, insulin sensitivity, indicators of systemic inflammation (including hsCRP, tumor necrosis factor-α, and transforming growth factor-β), and liver histology in patients with NASH and IGT or T2DM. Treatment with pioglitazone also ameliorated adipose, hepatic, and muscle IR, and was associated with an approximately 50% decrease in necroinflammation (p<0.002) and a 37% reduction in fibrosis (p=0.08).

Improvement in hepatocellular injury and fibrosis has been reported in another controlled trial with pioglitazone of 12 months duration. In contrast, while the first randomized clinical study with rosiglitazone, the other thiazolidinedione approved for diabetes treatment, in NASH demonstrated a reduction in IR, plasma alanine aminotransferase (ALT) levels and steatosis, rosiglitazone treatment had no significant effect on necrosis, inflammation, or fibrosis. It is important to note with these results that even reduced ALT, insulin resistance and other diabetes indicators did not decrease liver fibrosis, which is a key indicator of NASH. Therefore, controlling diabetes is not enough to treat NASH or even prevent NASH. Moreover, there are severe safety limitations with both pioglitazone and Rosiglitazone. A preliminary report of the 2-year, open-label follow-up of this trial was also disappointing, with no significant benefit from rosiglitazone treatment.

One pharmacological agent with some efficacy in NASH is pioglitazone. Unfortunately, pioglitazone is also associated with a significantly increased risk of weight gain, edema, congestive heart failure, and osteoporotic fractures in both women and men.

A phase 2 trial involving patients with NASH showed that treatment with daily subcutaneously-administered semaglutide (GLP-1 receptor agonist) resulted in a higher percentage of patients with NASH resolution than placebo. However, the trial did not show a significant between-group difference in the percentage of patients with an improvement in fibrosis stage (Newsome et al., N. Engl. J. Med. “A Placebo-Controlled Trial of Subcutaneous Semaglutide in Nonalcoholic Steatohepatitis” Nov. 13, 2020). Unfortunately, “[t]he percentage of patients in whom NASH resolution was achieved with no worsening of fibrosis was 40% in the 0.1-mg group, 36% in the 0.2-mg group, 59% in the 0.4-mg group, and 17% in the placebo group (P=0.48). The mean percent weight loss was 13% in the 0.4-mg group and 1% in the placebo group. The incidence of nausea, constipation, and vomiting was higher in the 0.4-mg group than in the placebo group (nausea, 42% vs. 11%; constipation, 22% vs. 12%; and vomiting, 15% vs. 2%). Malignant neoplasms were reported in 3 patients who received semaglutide (1%) and in no patients who received placebo. Overall, neoplasms (benign, malignant, or unspecified) were reported in 15% of the patients in the semaglutide groups and in 8% in the placebo group; no pattern of occurrence in specific organs was observed.” Accordingly, even GLP-1 agonists, such as semaglutide, are not benign treatments for NASH prevention or treatment to warrant the risk of long-term administration needed to treat, prevent or slow progression of NASH.

In Wilding et al., N. Engl. J. Med. Published 10 Feb. 2021, an obesity study was conducted with semaglutide at a maintenance dose of 2.4 mg administered subcutaneously once a week for 68 weeks (or placebo). “In the semaglutide group, weight loss was observed from the first post-randomization assessment (week 4) onward, reaching a nadir at week 60.” However, there were many side effects including “Gastrointestinal disorders (typically nausea, diarrhea, vomiting, and constipation) were the most frequently reported events and occurred in more participants receiving semaglutide than those receiving placebo (74.2% vs. 47.9%).” More concerning was that “[s]erious adverse events were reported in 9.8% and 6.4% of semaglutide and placebo participants, respectively.” More semaglutide participants discontinued due to severity of side effects.

In addition, the GLP-1 analogs exert GLP-1 activity and not GLP-2 activity, exert effects mainly via hormonal signaling pathways continuously and not in a normal episodic nature (consistent with episodic meals). In view of the continuous hormonal pathway stimulation, there are significantly increased side effect risk of thyroid c-cell tumors and pancreatitis. Further, frequently antibodies are formed against the synthetic GLP-1 analog derivatives (formed to prevent DPP-4 enzymatic degradation), for example, 61% of patients developed antibodies to exenatide. Therefore, there is a need in the art for better combinations with GLP-1 analogs to allow for lower GLP-1 analog dosing to address serious side effects observed with chronic dosing.

A summary of the clinical data obtained indicates that treatment of NASH seems to be uncoupled from weight loss as a treatment means, by any weight loss technique, even though weight loss may be an effective means for prevention of NASH or possibly slowing progression of NASH. Therefore, there is a need for better accepted translational models to predict prevention, preventing progression and treatment of fatty liver diseases, including NASH. Therefore, there is a need for effective and safer NASH treatment options, particularly if a treatment can be delivered orally and not by injection. There is also a need for safe agents to prevent development of full NASH liver disease and damage and to slow progression of NASH.

### SUMMARY

The present disclosure was made, in part, to address the foregoing needs in the treatment and/or management of glucagon-related diseases, disorders or conditions, including: (a) glycemic control/diabetes/metabolic syndrome (MetS), (b) weight loss and/or obesity, and (c) hyperlipidemia. Disclosed herein is the discovery of a combination of one or more bitter receptor agonists (otherwise referred to as TAS2R agonists), and at least one gut-signaling compound, that provides significant benefits and advantages over currently available treatments for glucagon-related conditions involving use of gut-signaling compounds (i.e., gut signaling peptide analogs and gut signaling hormone enhancers). The present disclosure was also made in part to address the foregoing needs in treating or preventing fatty liver disease such as NASH.

Preferably, the combination described herein comprising the bitter receptor agonist (e.g., with at least one gut-signaling compound) is formulated into a pharmaceutical composition, more preferably, an oral dosage form. The combination can provide significant advantages in treating glucagon-related diseases, disorders, and conditions, including, for example, diabetes, prediabetes syndrome, obesity, weight and/or appetite control, hyperlipidemia, and hyperglycemia. One advantage of the inventive combination is to produce the same or greater efficacy with reduced dosages of gut-signaling compounds, to achieve the same or better results with reduced side-effects.

The present disclosure further provides a method for treating or preventing progression of glucagon-related diseases, disorders, and conditions, for example, diabetes, prediabetes syndrome, obesity, weight and/or appetite control, hyperlipidemia, and hyperglycemia, comprising administering to a subject having such a disease, disorder, or condition, a combination of one or more bitter receptor agonists and a gut-signaling compound.

The present disclosure further provides a method for treating or preventing progression of fatty liver disease (e.g., selected from the group consisting of NASH, ASH, NAFLD, or HIV-associated steatohepatitis, with or without liver fibrosis), comprising administering to a subject having fatty liver disease, a combination comprising a denatonium salt, wherein the denatonium salt is selected from the group consisting of denatonium acetate (DA), denatonium citrate, denatonium maleate, denatonium saccharide, and denatonium tartrate; and a GLP-1 agonist drug. Preferably, the GLP-1 agonist drug is selected from semaglutide, glyburide, liraglutide, dulaglutide, and/or albiglutide.

In some embodiments, the daily dose of the denatonium salt for a human adult is from about 50 mg to about 3000 mg administered once per day (QD) or twice per day (BID). Preferably, the daily dose of the denatonium salt is from about 100 mg to about 2000 mg administered QD or BID. Most preferably, the daily dose of the denatonium salt is from about 200 mg to about 1000 mg administered QD or BID.

In some embodiments, the method further comprises administering acetic acid, e.g., from about 0.5 g to about 5 g per dose. More preferably, the dosage per day of the acetic acid for an adult is from about 1.5 g to about 3 g.

### BRIEF DESCRIPTION OF THE FIGURES

In the Figures, the designation “ARD-101” means denatonium acetate (DA).

FIG. 1 shows the average body weight gain across the study period for all treatment groups in Example 2.

FIG. 2 present serum triglyceride (TG) levels at the end of study (Day 31) for each animal in the four treatment groups in Example 2. These data show that (1) treatment with DA, liraglutide, or their combination significantly decreased serum TG level in diet-induced obese (DIO) mice as compared to vehicle; and (2) animals treated the combination showed a significantly lower serum TG level as compared to those treated with DA or liraglutide alone, indicating a potential synergistic (or at least additive) effect on serum TG level between the two agents.

FIG. 3 presents serum glucose level at the end of study (Day 31) for each animal in the four treatment groups in Example 2. Treatment with DA, liraglutide, or their combination significantly decreased serum glucose level in DIO mice upon 4-week dosing.

FIG. 4 shows serum HbA1c level at the end of study (Day 31) for each animal in the four treatment groups in Example 2. The results suggest that treatment with DA, liraglutide, or their combination did not show significant effect (p>0.05) on serum HbA1c level in DIO mice after 4-week dosing.

FIG. 5 depicts serum insulin level at the end of study (Day 31) for each animal in the four treatment groups in Example 2. The data reveal that 4-week treatment with DA, liraglutide, or their combination considerably decreased serum insulin level in DIO mice.

FIG. 6 presents serum BA level at the end of study (Day 31) for each animal in the four treatment groups in Example 2. Upon 4-week dosing, DA, liraglutide, or their combination resulted in a significant increase in serum BA level as compared to vehicle control.

FIG. 7 shows serum LDL level at the end of study (Day 31) for each animal in the four treatment groups in Example 2. There was no significant difference in serum LDL level among animals treated either with vehicle or with DA, liraglutide, or their combination.

FIG. 8 shows serum HDL level at the end of study (Day 31) for each animal in the four treatment groups in Example 2. The data reveal that as compared to vehicle, treatment with liraglutide or the combination of DA plus liraglutide led to a significant decrease in serum HDL level in DIO mice after 4-week dosing.

FIG. 9 shows the percent change for GLP-1 between time 0 and one hour after oral dosing in Example 3. In a small sample size, the difference was significant (p=0.0235).

FIG. 10 shows the results for GLP-2 in Example 3, which showed a trend for GLP-2 gut peptide hormone increase.

FIG. 11 shows the results for PYY in Example 3, which showed a trend for PYY gut peptide hormone increase.

FIGS. 12A and 12B show relative body weight percentage (12A) and relative body weight change (g) (12B) for the four groups of animals treated in Example 4. Treatment with sitagliptin alone showed the least effect on body weight, which is consistent with previous studies and clinical experience. However, much greater effect on body weight was seen with DA alone (ARD-101), but a considerable or synergistic effect on body weight was seen with the combination of DA and sitagliptin.

FIG. 13 shows that the combination of DA (ARD-101) and sitagliptin significantly lowered body weight gain in DIO mice at day 56 of the study in Example 4. The combination of DA and sitagliptin significantly lowered body weight gain in DIO mice as compared with mice treated with even sitagliptin at the same dose.

FIGS. 14A and 14B show that treatment with DA or its combination with sitagliptin, at day 56 of the study, both showed a significant effect on body weight in Example 4.

FIGS. 15A and 15B show that DA alone and DA plus sitagliptin significantly decreased fasting blood glucose levels in DIO mice as compared with vehicle controls in day 28 (FIG. 15A) and day 56 (FIG. 15B).

FIGS. 16A and 16B show that DA plus sitagliptin significantly decreased HbA1c levels in DIO mice as compared with vehicle controls at day 28 (FIG. 16A) and day 56 (FIG. 16B). The baseline day 0 HbA1c level was 4.7%.

FIGS. 17A and 17B show that DA plus sitagliptin significantly decreased insulin levels in DIO mice as compared with vehicle controls at day 28 (FIG. 17A) and day 56 (FIG. 17B). The baseline day 0 insulin level was 1 ng/ml.

FIGS. 18A and 18B show that DA plus sitagliptin significantly decreased triglyceride (TG) levels in DIO mice as compared with vehicle controls in day 28 (FIG. 18A) and day 56 (FIG. 18B). The baseline day 0 triglyceride level was 33.8 mmol/L.

FIGS. 19A and 19B show that DA plus sitagliptin significantly decreased bile acid (BA) levels in DIO mice as compared with vehicle controls in day 28 (FIG. 19A) and day 56 (FIG. 19B). The baseline day 0 bile acid level was 27 μmol/L.

FIGS. 20A and 20B show that DA plus sitagliptin significantly decreased total cholesterol (TC) levels in DIO mice as compared with vehicle controls in day 28 (FIG. 20A) and day 56 (FIG. 20B). The baseline day 0 total cholesterol level was 110 μg/μL.

FIGS. 21A and 21B show that DA plus sitagliptin significantly decreased low-density lipoprotein (LDL) levels in DIO mice as compared with vehicle controls in day 28 (FIG. 21A) and day 56 (FIG. 21B). The baseline day 0 low-density lipoprotein (LDL) level was 125 mg/dL.

FIG. 22A shows that sitagliptin alone significantly decreased high-density lipoprotein (HDL) levels as compared with vehicle controls.

FIG. 22B shows, however, that at day 56 sitagliptin alone, DA alone and DA plus sitagliptin significantly decreased high-density lipoprotein (HDL) levels in DIO mice as compared with vehicle controls. The baseline day 0 high-density lipoprotein (HDL) level was 60 mg/dL.

FIG. 23 shows that treatment with DA (ARD-101), semaglutide, or their combination significantly improved NAFLD Activity Score based on blinded histopathologic review.

FIGS. 24A and 24B show that treatment with DA (ARD-101), semaglutide or their combination showed a remarkable effect on body weight (24A) and body weight change (24B) in trans-fat containing amylin liver NASH (AMLN)-diet induced mice, including a synergistic combination. Data are presented as means. Statistical analysis was performed with one tailed t-test. *** P<0.001 as compared with vehicle; $$ P<0.01 and $$$ p<0.001 as compared with the combination.

FIGS. 25A and 25B show liver weight (FIG. 25A) and liver/body weight ratio (FIG. 25B) showing that: (1) both treatments significantly decreased liver weight and liver/body weight ratio as compared to vehicle; and (2) the effect of the combination of DA (ARD-101) and semaglutide was significantly greater compared to even single agent DA or semaglutide, indicating a synergistic effect between the two agents.

FIG. 26A shows alanine aminotransferase (ALT) levels.

FIG. 26B shows aspartate aminotransferase (AST) levels. At the end of the study, the two treatments each significantly decreased ALT and AST levels as compared to vehicle control. Moreover, the combination of DA (ARD-101) and semaglutide produced a significantly lower ALT level as compared with either DA or semaglutide alone, indicating a synergistic effect.

FIGS. 27A, 27B, and 27C show that at the end of the study of Example 6, DA (ARD-101) and semaglutide each significantly decreased TGs (27A), LDLs (27B) and HDLs (27C), respectively.

FIG. 28 shows that at the end of the study of Example 6, the combination of DA (ARD-101) and semaglutide significantly counteracted the increase in fasting glucose levels induced by the AMLN diet as compared to vehicle control.

FIG. 29 shows that at the end of the study the combination of DA (ARD-101) and semaglutide significantly increased HbA1c as compared to vehicle control. The baseline HbA1c level was 5.0%.

FIG. 30 shows that at the end of the study the combination of DA (ARD-101) and semaglutide significantly decreased insulin levels as compared to vehicle control. The baseline insulin level was 1.5 ng/ml.

FIG. 31 shows that the two treatments did not significantly impact bile acid levels as compared to vehicle control. The baseline bile acid level was 30 μmol/L.

FIGS. 32A (CK-18) and 32B (TGF-β) show that the two treatments each significantly decreased CK-18 levels compared to vehicle control (FIG. 32A) and only the combination of semaglutide and DA significantly decreased TGF-β1 levels compared to vehicle control. These data provide further evidence for a synergistic effect for these two agents.

FIGS. 33A and 33B show that at the end of the Example 6 study, the two treatments did not significantly impact IL-6 and TNF-α levels as compared to vehicle.

### DETAILED DESCRIPTION

The present disclosure is based, in part, upon in vivo and clinical studies (presented in the Examples herein) that found surprisingly beneficial and/or synergistic results in using a combination of a bitter receptor agonist, specifically an orally-administered denatonium salt, wherein the denatonium salt is selected from the group consisting of denatonium acetate (DA), denatonium citrate, denatonium maleate, denatonium saccharide, and denatonium tartrate; and at least one gut-signaling compound for treating glucagon-related diseases, disorders, or conditions, including weight control and fatty liver disease, and for preventing progression of a fatty liver disease.

Section headings are provided solely for the convenience of the reader and do not limit the disclosure.

To the extent any material incorporated by reference is inconsistent with the express content of this disclosure, the express content controls.

### Definitions

“About” as used herein includes the exact amount modified by the term, about, as wells as an amount that would be expected to be within experimental error, such as for example, within 15%, 10%, or 5%. For example, “about 5 mg” means “5 mg” and also a range of mgs that is within experimental error, e.g., plus or minus 15%, 10%, or 5% of 5 mg. As used herein, the term “about” may be used to modify a range and also, a particular value.

“Administering a combination” refers to any administration of a plurality of agents, whether the agents are administered simultaneously or sequentially; in the same composition or different compositions; and by the same route or by different routes.

“API” means active pharmaceutical ingredient.

A “fatty liver disease” means any of a group of diseases characterized by undesirable accumulation of fat in the liver, including nonalcoholic steatohepatitis (NASH), alcoholic steatohepatitis (ASH), non-alcoholic fatty liver disease (NAFLD), and HIV-associated steatohepatitis, with or without liver fibrosis.

“Glucagon-related” disease, disorder or condition, as used herein, means any undesired state in a subject that is mediated by the production, maintenance or metabolism of glucagon in a subject or by the glucagon regulatory cycle including any conditions that may be mediated by a gut-signaling compound.

“Gut-signaling compound” means a gut-signaling peptide analog and/or gut-signaling hormone enhancer such as, for example, compounds selected from GLP-1 receptor agonists (sometimes also referred to as GLP-1 agonists or GLP-1 analogs), GLP-2 analogs, PYY analogs, DPP-4 inhibitors, GIP analogs, and CCK analogs, as further described herein.

“Or” is used in the inclusive sense (equivalent to “and/or”) unless the context requires otherwise.

As used herein, “synergy” or “synergistic” is used to convey the beneficial effects of API combinations providing efficacy of multiple gut peptide hormone receptor signaling agonists rather than just the increase of a single gut peptide hormone. In some embodiments, synergy is shown in that combinations of APIs dosed during in vivo studies produced more than additive benefits. Without being bound by theory, one hypothesis is that the beneficial effects of API combinations is due to (a) episodic increases of gut-signaling hormones versus long-acting GLP-1 receptor agonists that have a high incidence of serious side effects that limit their uses; and/or (b) efficacy of multiple gut peptide hormone receptor signaling agonists rather than just the increase of a single gut peptide hormone′ and/or (c) a combination effect to episodically augment gut signaling peptides of long-acting baseline properties.

The term and symbol “% by weight” and “%” refers to the percentage by weight of the excipient and API and when used with reference to multi-layer tablets, refers to the “% by weight in each individual layer, e.g, the “individual layer” means the first layer or the second layer of the bilayer tablet.

A “therapeutically effective amount” of an API means an amount which, when administered to a human for treating a disease (for example fatty liver disease, such as NAFLD or NASH), is sufficient to effect treatment for the disease state being treated. As applied to NAFLD or NASH in a human, “treating” or “treatment” includes one or more of:
(1) preventing or reducing the risk of developing NAFLD or NASH, i.e., causing the clinical symptoms of NAFLD or NASH not to develop in a subject who may be predisposed to NAFLD or NASH but who does not yet experience or display symptoms of the NAFLD or NASH (i.e. prophylaxis);
(2) inhibiting NAFLD or NASH, i.e., arresting or reducing the development of NAFLD or NASH or its clinical symptoms; and
(3) relieving NAFLD or NASH, i.e., causing regression, reversal, or amelioration of the NAFLD or NASH or reducing the number, frequency, duration or severity of its clinical symptoms.

Similarly, “treating” or “treatment” as applied to T2DM, includes treating diabetes and preventing the onset of diabetes or progression of T2DM to require insulin treatment, by treating pre-diabetic conditions.

The therapeutically effective amount for a particular subject varies depending upon the health and physical condition of the subject to be treated, the extent of disease progression (e.g., the NAFLD or NASH), the assessment of the medical situation, and other relevant factors. It is expected that the therapeutically effective amount will fall in a relatively broad range that can be determined through routine trial.

### EMBODIMENTS

The present disclosure is based upon the surprising discovery of synergistic combinations in (a) an in vivo weight loss study of two groups of compounds with different mechanisms of action measuring gut peptide hormone levels, (b) a phase 1 clinical study with oral dosing which measured gut peptide hormones before dosing and one hour after dosing, and (c) a 56 day in vivo weight loss study in DIO mice showing synergy of a combination of two orally administered drugs over each drug administered alone. (a) The in vivo study (see Example 2 for results) was a chronic weight control study with DIO mice to investigate synergistic effects as between a denatonium salt (denatonium acetate or DA) and the GLP-1 receptor agonist liraglutide. These surprising findings are reflected in the gut peptide hormones, GLP-1, CCK, PYY and GLP-2, and standard blood tests such as HbA1c and lipids.

A phase 1 clinical trial that administered denatonium acetate orally (see Example 3) resulted in an increase in gut hormonal signaling of GLP-1 and two additional gut hormone peptides. The gut peptide hormone data from the clinical trial administering DA showed a possible mechanism of action for weight loss of denatonium acetate is based upon signaling via multiple gut hormone peptides as the pharmacokinetic data showed that DA was primarily gut restricted and did not affect weight loss through DA systemic concentrations because PK (pharmacokinetic) analysis showed that DA was substantially gut restricted. Therefore, combinations of a denatonium salt with other gut peptide agonists, such as GLP-IRAs, GIP analogs, PYY analogs and DPP-4 inhibitors which act to increase plasma half-life of gut signaling peptides GLP-1, PYY and CCK, can significantly augment their activity and allow for lowering doses of GLP-IRA to reduce side effects. Accordingly, the gut peptide hormone data in both the in vivo studies in the examples herein and the phase 1 clinical trial data in Example 3 show synergy for the treatment and/or management of glucagon-related diseases, disorders or conditions, various indications. The clinical data showed there are multiple gut peptide hormones (not just GLP-1) that DA impacted. The clinical data also corroborated the DIO mouse data (Example 2). Yet, the marketers of GLP-1 agonists (like those available from Novo Nordisk, Lilly) claim only GLP-1 is important for both diabetes and weight loss. Also, DPP-4 is an enzyme that degrades GLP-1 and PYY to give both of those hormones short half-lives. Accordingly, in some embodiments herein, a DPP-4 inhibitor is used as part of the API combination.

In one embodiment herein, the bitter receptor agonist (or TAS2R agonist) is substantially gut-restricted and exerts its activity through gut peptide hormones. DPP-4 inhibitors do not provide meaningful weight loss benefits. A 56-day in vivo weight loss study in DIO mice showed synergy of a combination of two orally administered drugs (DA, a bitter receptor agonist that is substantially gut-restricted, and the DPP-4 inhibitor sitagliptin phosphate) over each drug administered alone. Sitagliptin phosphate (Januvia®) produced slight weight loss over the initial 30 days of dosing, but as seen with patients, the weight returned, and no weight loss effect was seen with longer duration dosing. Therefore, sitagliptin phosphate showed its well-known lack of weight loss effects. DA produced significant weight loss. But adding sitagliptin, with no significant weight loss effect on its own, significantly increased the weight loss benefit of DA. This synergistic effect was also seen in other measured metabolic parameters measured as well, including HbA1c, insulin, triglycerides, blood glucose, bile acids, cholesterol and low-density lipoprotein (LDL). The data is presented in Example 4.

The data disclosed herein indicate that a combination of a bitter receptor agonist with either or both of a GLP-1RA (such as liraglutide or semaglutide) and a DPP-4 inhibitor and optionally a GIP agonist can (1) augment gut peptide hormone effectiveness, and (2) can allow for possible lower dosing of difficult (with severe side effects) gut peptide hormone agents (such as semaglutide or other GLP-1 agonists) to mitigate side effects, while providing for superior efficacy over each individual therapeutic component alone at a higher dose.

More specifically, the findings show that a combination of a bitter receptor agonist is synergistic with or adds “benefit” to a gut peptide hormone agent selected from the gut peptide analogs GLP-1, GLP-2, PYY, CCK, and DPP-4 inhibitors (that increase the half-life of natural gut peptide hormones GLP-1 and PYY). By “benefit” it can mean the ability to reduce the dosage of a GLP-1 agonist which can significantly mitigate many of the severe side effects of GLP-1 agonist administration that are indicated in product labeling. From a mechanism of action perspective, the denatonium salts are bitter receptor agonists and stimulate episodic and endogenous secretion of multiple gut peptides hormones (such as GLP-1, GLP-2, PYY, and CCK), which provide multiple gut axis signals (i.e., a symphony orchestra) instead of only one gut peptide hormone, such as GLP-1 (i.e., a violin) which is only one of the signals.

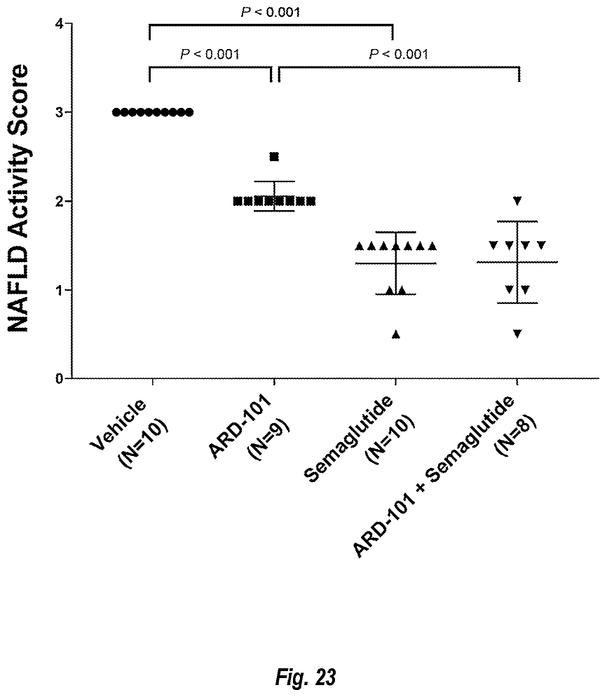

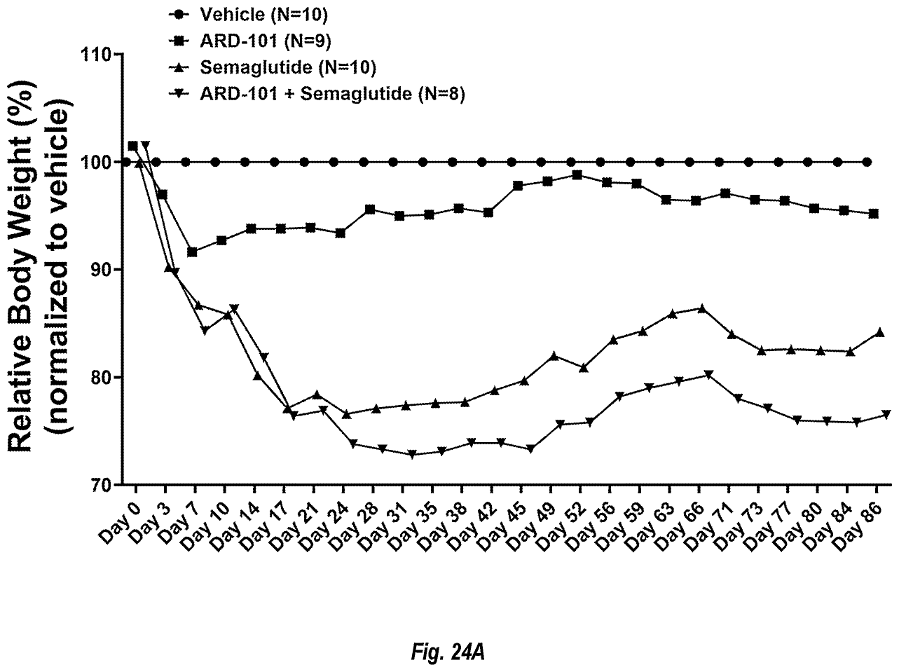

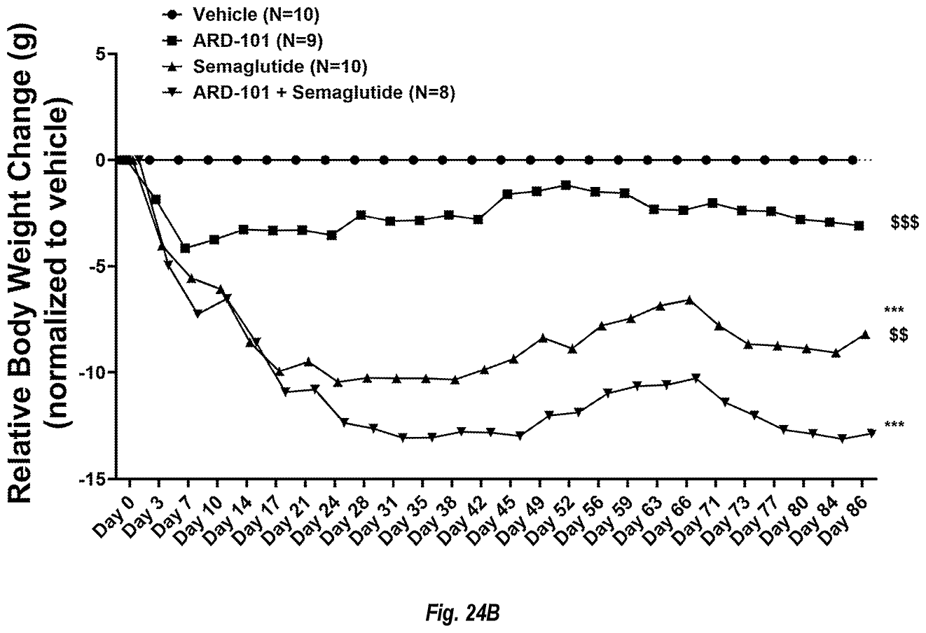

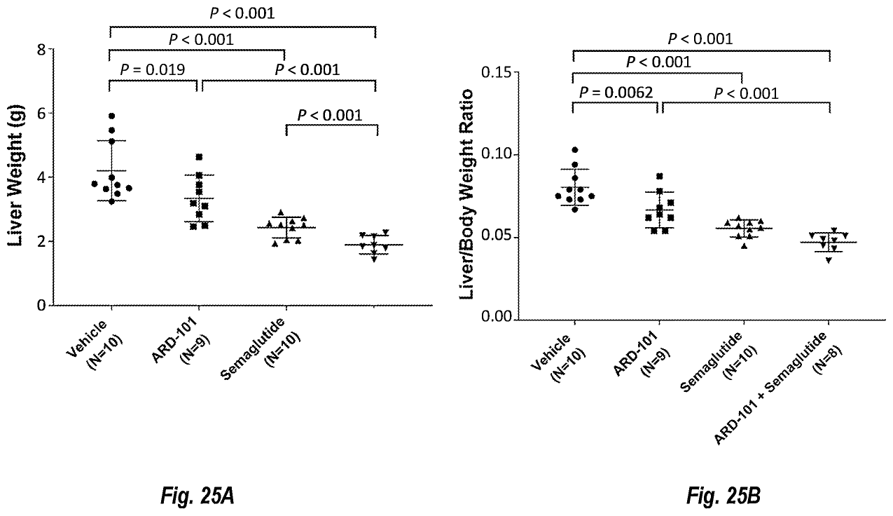

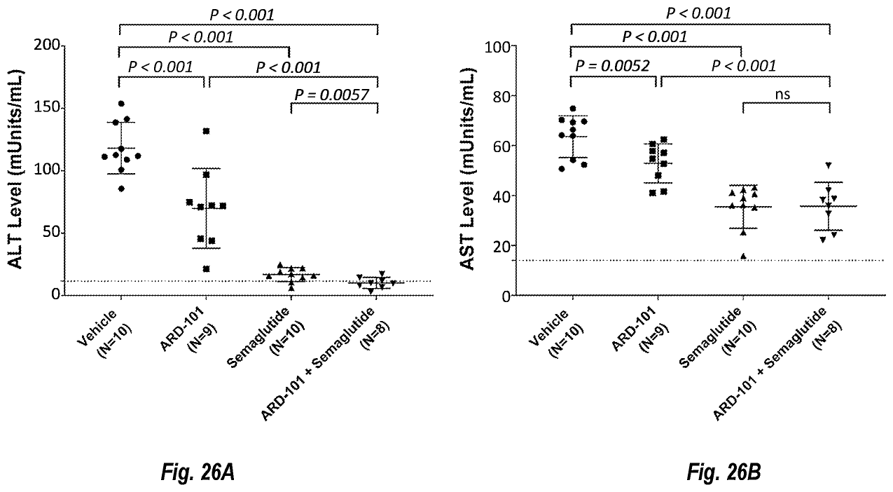

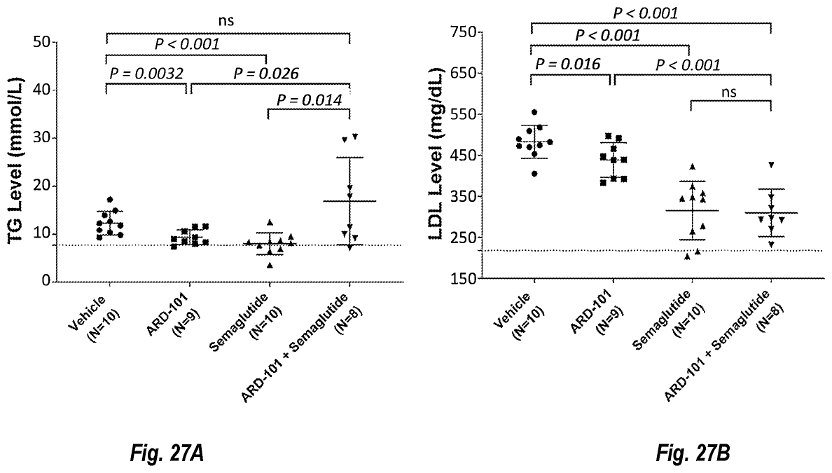

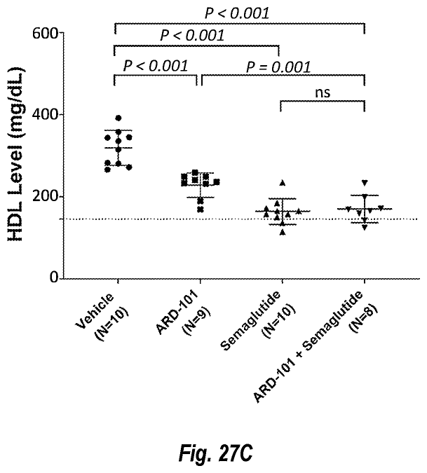

The data as disclosed herein further shows that the disclosed combination of a bitter receptor agonist and gut-signaling compound produces surprisingly beneficial results in treating fatty-liver disease such as ASH, NASH, and NAFLD. For example, following the in vivo study described in Example 6, the combination of DA and semaglutide significantly improved NAFLD Activity Scores (FIG. 23); showed a remarkable, synergist effect on body weight and body weight change in trans-fat containing amylin liver NASH (AMLN)-diet induced mice (FIGS. 24A, 24B); significantly decreased liver weight and liver/body weight ratios as compared to vehicle, with the combination of DA and semaglutide producing a significantly greater effect as compared to even single agent DA or semaglutide, indicating a synergistic effect with the combination of two agents (FIG. 25). The data further show, inter alia, that the inventive combination had a surprisingly improved affect in decreasing ALT and AST levels (FIG. 26), and decreasing TGs, LDLs, and HDLs, in the AMLN-diet induced mice (FIG. 27A-FIG. 27C).

Given the aforementioned findings and discoveries further described below, the present disclosure provides, in one embodiment, a combination pharmaceutical composition comprising a formulation of a bitter receptor agonist and a gut signaling compound such as a gut signaling peptide analog and/or gut signaling hormone enhancer. Preferably, the pharmaceutical combination further comprises a DPP-4 inhibitor, which acts to inhibit DPP-4 enzyme activity to break down endogenous GLP-1 and PYY gut peptide hormones.

In another embodiment, the present disclosure provides a combination oral dosage form pharmaceutical composition comprising a bitter receptor agonist and a DPP-4 inhibitor.

In another embodiment, the present disclosure provides a synergistic method for treating glucagon-related diseases, disorders or conditions, such as obesity, diabetes, glycemic control, metabolic syndrome, hyperlipidemia, and effecting weight loss, comprising administering an effective amount of a pharmaceutical composition comprising a bitter receptor agonist and one or more gut-signaling compounds. Preferably, the method further comprises administering an enhancer of endogenous GLP-1 and PYY activity—a DPP-4 inhibitor.

In another embodiment, there is described a synergistic method for treating multiple aspects of metabolic syndrome, including obesity, diabetes/MetS, and hyperlipidemia, comprising administering a DPP-4i and DA dosed concomitantly in a single dosage form or in separated dosage forms. There is no additive toxicity noted.

In another embodiment, the present disclosure provides a method for treating hyperlipidemia comprising administering an effective amount of an orally administered pharmaceutical composition comprising a combination of a bitter receptor agonist and a gut signaling compound.

In another embodiment, the present disclosure provides a method for treating glycemic control, metabolic syndrome (MetS) and diabetes comprising administering an effective amount of an orally administered pharmaceutical composition comprising a combination of a bitter receptor agonist and a gut signaling compound.

In another embodiment, the present disclosure provides a method for treating obesity and effecting weight loss, comprising administering an effective amount of an orally administered pharmaceutical composition.

The present disclosure further provides a method for treating MetS and diabetes comprising administering an effective amount of an orally administered pharmaceutical composition comprising a comprising a combination of a bitter receptor agonist and a gut signaling compound.

In another embodiment, the present disclosure further provides a method for treating fatty-liver disease, including ASH, NASH, and NAFLD (more preferably, for treating NASH), comprising administering an effective amount of an orally administered pharmaceutical composition comprising a comprising a combination of a bitter receptor agonist and a gut signaling compound. In one preferred embodiment, the disclosure provides a method for treating NASH comprising administering the combination of DA and a gut-signaling compound, more preferably wherein the gut-signaling compound is selected from a GLP-1 agonist, even more preferably, wherein the gut-signaling compound is semaglutide.

Preferably, in each of the embodiments herein, the bitter receptor agonist is selected from the group consisting of denatonium salts (including DA, denatonium citrate, denatonium maleate, denatonium saccharide, and denatonium tartrate) chlorpheniramine, diphenidol, famotidine, haloperidol, quinine, parthenolide, and aristolochic acid. More preferably, the bitter receptor is a denatonium salt selected from DA, denatonium citrate, denatonium maleate, denatonium saccharide, and denatonium tartrate, even more preferably DA. It is to be understood that these preferred selections for the bitter receptor agonist apply to each of the alternative embodiments and methods of use described herein, including the inventive combination pharmaceutical compositions and methods of use and treatment or prevention of glucagon-related diseases, disorders or conditions, and/or fatty-liver diseases including NASH, ASH and NAFLD.

In each of the embodiments disclosed herein, the DPP-4 inhibitor is selected from the group consisting of a salt of a medium chain fatty acid, a salt of N-(8-(2-hydroxybenzoyl)amino)caprylic acid, sodium N-(8-(2-hydroxybenzoyl)amino)caprylate (SNAC), sitagliptin salts (including phosphate salt), saxagliptin, linagliptin, alogliptin, and combinations thereof. Preferably, in one embodiment, the DPP-4 inhibitor is selected from sodium N-(8-(2-hydroxybenzoyl)amino)caprylate (SNAC), sitagliptin phosphate, saxagliptin, linagliptin, and alogliptin.

In one embodiment, the DPP-4 inhibitor is sitagliptin phosphate, combined in a single oral dosage form or taken together in two oral dosage forms.

Preferably, a dosage for a commercially approved DPP-4 inhibitor, used in the methods and combinations described herein, is at a daily dose that is an approved daily dose for the specific DPP-4 inhibitor that is administered once per day (QD) or twice per day about 8 hours apart (BID) and the bitter receptor agonist (preferably, denatonium salt) is administered one per day or twice per day at a total daily dose (per weight of denatonium) of from about 200 mg to about 480 mg.

For example, in one embodiment, when the DPP-4 inhibitor is sitagliptin, the total daily dose for a human adult is from about a 200 mg to about 1000 mg per day administered either QD or BID administered either QD or BID at the same time as the DPP-4 inhibitor or after the DPP-4 inhibitor. DA is preferably administered BID irrespective if the DPP-4 inhibitor is administered QD or BID. Preferably, a single dosage form comprises a ratio in an oral (PO) dosage selected from the group consisting of Sitagliptin 50 mg/DA 200 mg PO BID, Sitagliptin 50 mg/DA 240 mg PO BID, Sitagliptin 100 mg/DA 200 mg PO QD, Sitagliptin 100 mg/DA 240 mg PO QD, Sitagliptin 100 mg/DA 480 mg PO QD.

In an additional embodiment, the combination pharmaceutical composition further comprises an oral dosage form of a GLP-1RA semaglutide.

In the inventive combinations and methods described herein, the bitter receptor agonist may be administered in a single dosage form or in two dosage forms.

In the inventive combinations, methods and uses described herein, the gut-signaling compound is preferably selected from a GLP-IRA analog GLP-1 receptor agonist, a GLP-2 analog, a PYY analog, a DPP-4 inhibitor, a GIP analog, and a CCK analog.

More preferably:
1) the GLP-1RA is selected from the group consisting of semaglutide, glyburide, liraglutide, dulaglutide, and albiglutide;
2) the PYY 1875 analog is selected from the group consisting of NN-9775 (Novo Nordisk) and JNJ-9321 (Johnson & Johnson);
3) the CCK analog is selected from the group consisting of C-2819 (Astra Zeneca), NN-9056 (Novo-Nordisk), and A-71378 (AbbVie);
4) the DPP-4 inhibitor is selected from the group consisting of sitagliptin phosphate, vildagliptin, linagliptin, alogliptin, saxagliptin (BMS47718), P93/01 (Prosidion), SYR322 (Takeda), GSK 823093, Roche 0730699, TS021 (Taisho), E3024 (Eisai), and PHX-1149 (Phenomix); and
5) the GLP-2 analog is selected from the group consisting of teduglutide, glepaglutide, apraglutide, elsiglutide, HM-15912 (Hamni Pharmaceuticals), ZP-7570 (Zealand Pharma AS), GLP-2-ELP (PhaseBio Pharmaceuticals) MOD-1501 (OPKO Health), and HL-06 (Huons Global Co. Ltd.).

In one embodiment, the present disclosure provides a method to lower a dose administered of a GLP-1RA comprising co-administering a bitter receptor agonist, as described herein. Preferably, the GLP-1RA is selected from the group consisting of semaglutide, glyburide, liraglutide, dulaglutide, and albiglutide.

Regarding indications beyond obesity and weight loss, improvement of parameters represented by translationally relevant biomarkers (blood glucose, HbA1C, insulin, triglycerides, LDL cholesterol, and total cholesterol) showed that DA or its combination with sitagliptin demonstrated superior benefit relative to sitagliptin alone.

Sitagliptin is renally excreted with minimal liver metabolism (via CYP3A4 and CYP2C8) whereas DA (Example 3) exhibited about ˜99% gut-restricted with limited systemic exposure. Therefore, both the pharmacokinetic data in a clinical trial (Example 3) for DA and the pharmacokinetic data published for commercially-marketed sitagliptin, support non-cumulative toxicity risk. Accordingly, DPP-4i drugs and the denatonium salts listed herein are an effective oral combination for the treatment of obesity.

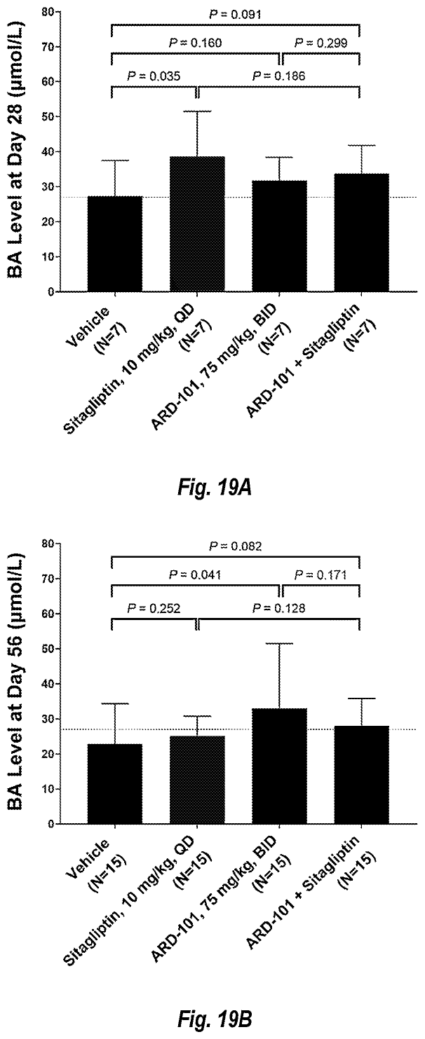

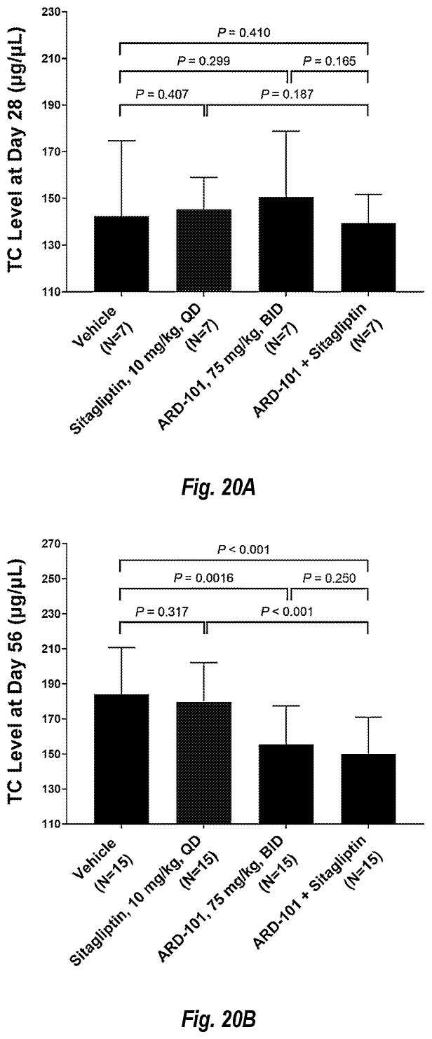

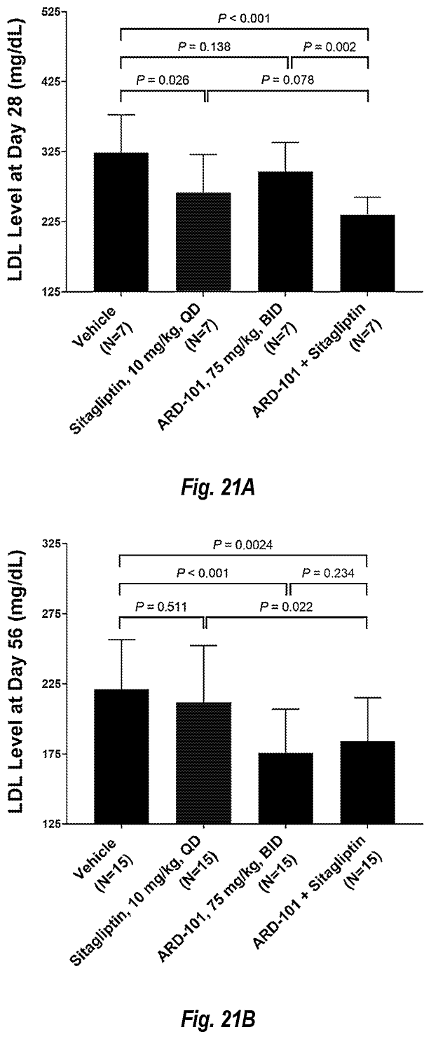

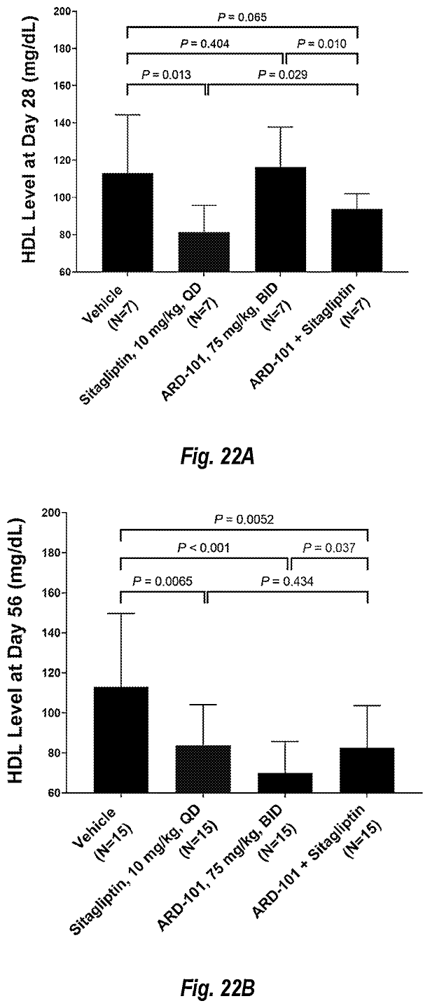

These data support the finding that beyond obesity, DA alone or in combination with DPP-4i provides super benefit for the treatment of diabetes as well as metabolic syndrome in general. Sitagliptin, beyond its primary intended effect on blood glucose, is not known to be as effective in addressing hyperlipidemia (cholesterol and triglycerides) even though hyperlipidemia is an important frequent co-morbidity with diabetes or obesity. However, as FIGS. 20-23 show, sitagliptin in combination with DA showed efficacy for hyperlipidemia.

Table 1 compares the data provided in Example 4, below, for DA plus sitagliptin combination therapy with 4 marketed GLP-1 RAs (exenatide, dulaglutide, liraglutide and semaglutide), in similar and comparable published in vivo studies.

TABLE 1

| | | DA plus | | | | |
|---|---|---|---|---|---|---|
| Product Name | | sitagliptin | Exenatide | Dulaglutide | Liraglutide | Semaglutide |
| Animal Model | | Male C57BL/6N | 8-week-old | 4-week-old | Male C57BL/6 | Male C57BL/6 |
| (No. per group) | | Tac mice (12- | male C57BL/6 | male C57BL/6 | mice fed on | mice (8-week |
| | | 14 weeks old | mice fed on | mice fed on | HFD for 30 | old) fed on |
| | | at study start) | HFD for 12 | HFD for 12 | weeks before | HFD for 18 |
| | | fed with HFD | weeks before | weeks | study started | weeks before |
| | | (N = 15) | study started | (N = 7) | (N = 10) | study started |
| | | | (N = 6) | | | (N = 6) |
| Dose and | | DA: 75 mg/kg | 24 nmol/kg | 0.6 mg/kg, | 0.3 mg/kg, QD, | 25 nmol/kg |
| Schedule | | (active moiety | (0.1 mg/kg), | once weekly, | SC, for 38 | (0.1 mg/kg), |
| (HED)^{#} | | weight), | QD, IP, for | IP for 12 | days | every two days, |
| | | BID, PO for | 4 weeks | weeks | (1.46 mg, QD) | SC, for 3 |
| | | 8 weeks | (0.49 mg, QD) | (3 mg, | | weeks |
| | | (360 mg, BID) | | once weekly) | | (0.49 mg, |
| | | Sitagliptin: | | | | every two days) |
| | | 10 mg/kg | | | | |
| | | (active moiety | | | | |
| | | weight), | | | | |
| | | QD, PO for | | | | |
| | | 8 weeks | | | | |
| | | (50 mg, QD) | | | | |
| Clinical | | DA: 240 mg, | 10 μg, | 1.5 mg, | 1.8 mg, | 2.4 mg, |
| Highest | | BID, PO | BID, SC | once weekly, | QD, SC | once weekly, |
| Dose | | Sitagliptin: | | SC | | SC (WEGOVY ®) |
| | | 100 mg, | | | | |
| | | QD, PO | | | | |
| Efficacy | Control- | −13.3%*** | −18.8%* | −15.9%* | −12.1%* | −13.4%^{##} |
| at end | adjusted | | | | | |
| of study | Percent | | | | | |
| | Change | | | | | |
| | in Body | | | | | |
| | Weight | | | | | |
| | Control- | −5.84 g*** | −7.07 g* | −8.1 g* | −7.2 g* | N/A |
| | adjusted | | | | | |
| | Body | | | | | |
| | Weight | | | | | |
| | Gain | | | | | |
| | Control- | −6.6%** | −25.7%* | −16.3%* | −22.9%*** | −12.8%*** |
| | adjusted | | | | | |
| | Percent | | | | | |
| | Change in | | | | | |
| | Fasting | | | | | |
| | Blood | | | | | |
| | Glucose | | | | | |
| | Control- | −58.0%*** | N/A | −77%* | −66.3%*** | N/A |
| | adjusted | | | | | |
| | Percent | | | | | |
| | Change in | | | | | |
| | Insulin | | | | | |
| | Level | | | | | |
| | Control- | TG: −8.3% | TG: −23.1%* | N/A | N/A | TG: −13.8%** |
| | adjusted | TC: −18.5%*** | TC: −11.9%* | | | TC: −16.7%** |
| | Percent | LDL: −16.8%** | | | | LDL: −9.3%* |
| | Change | | | | | |
| | in Lipid | | | | | |
| | Levels | | | | | |

Table 1 shows a comparison between a combination of DA and sitagliptin versus various GLP-1 agonists for various parameters. #Based on a body weight of 60 kg; ##Absolute body weight percent change *, **, and *** represent p<0.05, <0.01, and <0.001 vs. control, respectively. Abbreviations: HFD, high-fat diet; HED, human equivalent dose; BID, twice daily; QD, once daily; HbA1c, hemoglobin A1C; TG, triglyceride; TC, total cholesterol; LDL, low-density lipoprotein; PO, oral; SC, subcutaneous; IP, intraperitoneal. References: Sci Rep. 2019; 9(1):15601; Int. J. Obes. (Lond). 2020; 44(4):937; Eur. J. Med. Chem. 2020; 198:112389; Sci. Transl. Med. 2018; 10(472): eaat3392.

GLP-1RA drugs, such as semaglutide, also work along the GLP-1 axis but as they utilize a GLP-1 like structure that is not subject to quick endogenous degradation. However, the drawbacks of GLP-1RA are that they generally must be injected (with the exception of approved oral semaglutide Rybelsus®) and have a “black box” warning due to increased associated risk of cancer and pancreatitis. One of the more notable advantages of GLP-1RAs, in contrast to DPP-4i drugs, is their effect on weight loss, which has not been replicated consistently by oral DPP-4i drugs. The degree of measured weight loss relative to controls has been 10-15%, but over a much longer time period than the duration of the DA/DPP-4i combo study referenced in this application. Table 1 shows a comparison of data in predictive in vivo models as between DA plus DPP-4i compared to GLP-1RA agents. Therefore, given the magnitude of measured improvement relative to control, it would appear DA plus sitagliptin (or another DPP-4i drug) shows similar efficacy and superior safety to GLP-IR agonists for the treatment of obesity, diabetes, or metabolic syndrome in general.

Preferably, dosing in humans uses current optimal doses of both a DPP-4i agent and DA at their suggested dose as single agents administered concomitantly once daily or twice daily. For sitagliptin as a single agent, the current guidelines indicate a daily dose of 100 mg PO QD (notwithstanding for those with renal impairment, recommended doses can be as low as 25 mg to 50 mg PO QD). DA is undergoing clinical trials and has been shown in a phase 1 clinical trial. provided in Example 3 herein, to be safely dosed to 240 mg PO BID. In view of DA's pharmacokinetics (substantial gut restriction) and relatively non-toxic nature, the optimal dose ranges can be safely adjusted higher. Ongoing Phase 2 clinical trials are using 200 mg DA PO BID. DA may be taken once per day or twice per day.

Accordingly, several doses for a combination tablet/capsule formulation with both sitagliptin phosphate (100 mg total dose per day) and DA (from about 200 mg to about 1000 mg total dose per day based on the weight of denatonium) are the following:
1. Sitagliptin 50 mg/DA 200 mg PO BID
2. Sitagliptin 50 mg/DA 250 mg PO BID
3. Sitagliptin 100 mg/DA 200 mg PO QD
4. Sitagliptin 100 mg/DA 400 mg PO QD
5. Sitagliptin 100 mg/DA 500 mg PO QD.

DA is preferably dosed BID because as the observed appetite suppression effect in animals has been shown to last about 8 hours. Thus, twice daily dosing is preferred for abrogation of appetite throughout the day. However, additional data have demonstrated that when even DA is dosed once daily (and at lower levels than each BID dose in prior studies in the NASH studies), DA confers metabolic benefit independent of weight loss. Therefore, the preferred dosing for MetS treatment is QD.

Both QD and BID dosing are effective for applications in metabolic syndrome. However, if obesity is a primary indication for treatment, BID dosing is the preferred embodiment. And if other aspects of metabolic syndrome (diabetes and hyperlipidemia) are the primary indications of treatment either QD or BID dosing may be preferred (QD for convenience and patient compliance).

### GLP-1 Receptor Agonists

The class of GLP-1 receptor agonists (sometimes also referred to simply as GLP-1 agonists or GLP-1 analogs) includes: Dulaglutide (Trulicity®), which may be taken by injection weekly; liraglutide (Victoza®), which may be injected daily, Exenatide extended release semaglutide (Bydureon®), which may be taken by injection weekly; Exenatide ER (Astra Zeneca), which may be taken by injection weekly; Semaglutide (Ozempic®), which may be taken by injection weekly; Semaglutide (Rybelsus®), which may be taken by mouth once daily; Lixisenatide (Adlyxin®), which may be taken by injection daily; and albiglutide (Tanzeum®), which may be injected weekly. The Novo-Nordisk GLP-1 analogs semaglutide and liraglutide are fatty acid-modified GLP-1 protein receptor agonists. Dulaglutide and albiglutide from Lilly and GSK, respectively, are fusion protein GLP-1 receptor agonists.

GLP-1 analogs are approved for the treatment of type 2 diabetes as measured by glycemic control (HbA1c). GLP-1 analogs are also now being evaluated in clinical trials for weight loss and obesity. GLP-1 induces numerous biological effects such as stimulating insulin secretion, inhibiting glucagon secretion, inhibiting gastric emptying, inhibiting gastric motility or intestinal motility, and inducing weight loss. A characteristic of GLP-1 is its ability to stimulate insulin secretion without the associated risk of hypoglycemia that is seen when using insulin therapy or some types of oral therapies that act by increasing insulin expression.

GLP-1/glucagon receptor co-agonists are disclosed in WO2008/086086, WO2008/101017, WO2007/056362, WO2008/152403 and WO96/29342. Other glucagon analogs disclosed are PEGylated (WO2007/056362) or acylated in specific positions of native human glucagon (WO96/29342). Glucagon peptides have been disclosed in U.S. Pat. No. 7,314,859. The disclosures of each of the foregoing GLP-1 analogs are incorporated by reference herein.

Liraglutide is an analog of human GLP-1 and acts as a GLP-1 receptor agonist. It is indicated for the treatment of patients with type 2 diabetes to improve glycemic control. U.S. Pat. No. 6,268,343 discloses liraglutide and its formulations. U.S. Pat. No. 8,114,833 discloses a pharmaceutical formulation comprising a GLP-1 receptor agonist, a disodium phosphate dihydrate buffer, and propylene glycol, wherein the propylene glycol is present in the formulation in a final concentration of from 1 mg/mL to 100 mg/mL, and wherein the formulation has a pH of from 7.0 to 10.0. U.S. Publication 2010/0234299 discloses a pharmaceutical formulation of a GLP-1 compound, an isotonic agent, a buffer, and a preservative, wherein the formulation has a pH of from 7.0 to 10.0 and provides that if an isotonic agent is present and the pH of the formulation is 7.4, then mannitol or NaCl is not the isotonic agent.

GLP-1 analogs are either short-acting or long-acting, which require different dosing schedules. However, normal physiology experiences episodic GLP-1 bolus, triggered by meals, and not long term or steady-state GLP-1 gut hormone stimulation. Table 2 provides a list of long and short-acting GLP-1 analogs.

Injectable GLP-1 agonists, like semaglutide (up to 2 mg injectable) can cause series side effects including medullary thyroid carcinoma, renal inflammation, pancreatic inflammation, changes in vision, gallbladder and serious allergic reactions, including angioedema. The serious side effects are dose-related. Therefore, the disclosed combination with an oral denatonium salt allows for use of a lower and safer dose of a GLP-1 agonist to provide a safer treatment option for the existing approved indications of GLP-1 agonists (lowering HbA1C, weight loss, glycemic control).

TABLE 2

| | | | Long-acting GLP-1 Agonists | | | |
|---|---|---|---|---|---|---|
| | Short-acting GLP-1 Agonists | | | Extended-release | | |
| | Exenatide | Lixisenatide | Liraglutide | Exenalide | Dulaglutide | Semaglutide |
| Dose schedule | Twice daily | Once daily | Once daily | Once weekly | Once weekly | Onca weekly |
| Incidence of Hypoglycemic | 3.8% | 2% | N/A | 2.1% | N/A | 3.8% |
| Episodes for Monotherapy at | (1.3%) | (2%) | | | | (0%) |
| the Highest Dose | | | | | | |
| (vs. Placebo) | | | | | | |
| Incidence of Hypoglycemic | 5.3% | 3% | 3.6% | 0% | 0.7% | N/A |
| Episodes for Combination | (5.3%) | (1%) | (2.5%) | | (0%) | |
| with Metformin | | | | | | |
| (vs. Placebo) | | | | | | |
| Incidence of Hypoglycemic | N/A | 28%* | N/A | N/A | 14.7%* | 29.8%* |
| Episodes for Combination | | (23%) | | | (9.3%) | (15.2%) |
| with Insulin | | | | | | |
| (vs. Placebo) | | | | | | |
| Incidence of Hypoglycemic | 35.7% | 15%** | 7.5% | 25% | 3.3% | N/A |
| Episodes for Combination | (3.3%) | (11%) | (2.6%) | | (0%) | |
| with a Sulfonylurea | | | | | | |
| (vs. Placebo) | | | | | | |

*Add-on to basal insulin with or without metformin.
**Add-on to a sulfonylurea with or without metformin. All data are from prescribing information of each product.

### DPP-4 Inhibitor

DPP-4 inhibitors are used along with diet and exercise to lower blood sugar in adults with type 2 diabetes. When untreated or under-treated, or even well-treated, type 2 diabetes can lead to serious problems, including blindness, nerve and kidney damage, and heart disease. DPP-4 inhibitors are available as single-ingredient products and in combination with metformin. Available DPP-4 inhibitors are sitagliptin, saxagliptin, vildagliptin, linagliptin, and alogliptin. However, when used alone, DPP-4 inhibitors are known to possibly cause joint pain that can be severe and disabling. For example, oral administration of vildagliptin or sitagliptin to human Type 2 diabetics has been found to reduce fasting glucose and postprandial glucose excursion in association with significantly reduced HbA1c levels.

DPP-4 inhibitors act by inhibiting the degradation GLP-1, GLP-2, and PYY all of which have intrinsically short half-lives. DPP-4 inhibitors have no effect on gastric emptying, are body weight neutral, and have a minor or barely perceptible effect on appetite. Therefore, DPP-4 inhibitors are indicated for only diabetes/glycemic control and not for weight loss, obesity, or hyperlipidemia. Reviews on the application of DPP-4 inhibitors for the treatment of Type 2 diabetes include: (1) Demuth, et al., “Type 2 diabetes-Therapy with dipeptidyl peptidase IV inhibitors, Biochim. Biophys. Acta, 1751: 33-44 (2005) and (2) Augustyns et al., “Inhibitors of proline-specific dipeptidyl peptidases: DPP-4 inhibitors as a novel approach for the treatment of Type 2 diabetes,” Expert Opin. Ther. Patents, 15: 1387-1407 (2005).

Sitagliptin phosphate is formula I below is the dihydrogenphosphate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine.

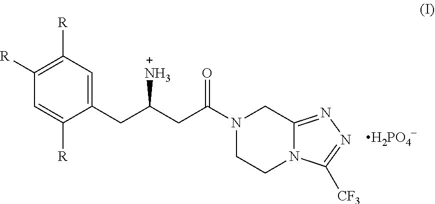

DPP-4 inhibitors are used in combination with another glycemic drug such as metformin hydrochloride (U.S. Pat. No. 8,414,921). However, there is a need to find synergistic or additive combinations with bitter agonists, such as denatonium salts based on increasing serum half-life of GLP-1 and PYY(1-36).

In terms of combination therapies, the GLP-RA semaglutide in oral form was compared to oral DPP-4 inhibitors in Table 3. Standard metric of glycemic control, weight change and serum lipids were compared based on published results at different doses of each marketed drug.

TABLE 3

| | | | | Vildagliptin | | |
|---|---|---|---|---|---|---|
| Product | Semaglutide | Sitagliptin | Saxagliptin | (Galvus ®) | Alogliptin | Linagliptin |
| Name | (Rybelsus ®) | (Januvia ®) | (Onglyza ®) | (not in U.S.) | (Nesina ®) | (Tradjenta ®) |
| Indication | Type 2 | Type 2 | Type 2 | Type 2 | Type 2 | Type 2 |
| | Diabetes | Diabetes | Diabetes | Diabetes | Diabetes | Diabetes |
| Dose and | 3 mg, 7 mg, and | 100 mg and | 2.5 mg, 5 mg, | 50 mg and 100 | 12.5 mg and 25 | 5 mg, QD |
| Schedule | 14 mg, QD for | 200 mg, QD | and 10 mg, QD | mg, QD for 24 | mg, QD for 26 | for 24 weeks |
| | 26 weeks | for 24 weeks | for 24 weeks | weeks | weeks | |
| Patient No. | 3 mg (N =175) | 100 mg (N = 238) | 2.5 mg (N = 102) | 50 mg (N = 177) | 12.5 mg (N = 133) | 5 mg (N = 183) |
| | 7 mg (N = 175) | 200 mg (N = 250) | 5 mg (N = 106) | 100 mg (N = 185) | 25 mg (N = 131) | Placebo (N = 89) |
| | 14 mg (N = 175) | Placebo (N = 253) | 10 mg (N = 98) | Placebo (N = 182) | Placebo (N = 65) | |
| | Placebo (N = 178) | | Placebo (N = 95) | | | |
| Effect on | 3 mg: −0.7% | 100 mg: −0.79% | 2.5 mg: −0.62% | 50 mg: −0.7% | 12.5 mg: −0.54% | 5 mg: −0.57% |
| HbA1c | 7 mg: −1.2% | 200 mg: −0.94% | 5 mg: −0.65% | 100 mg: −1.1% | 25 mg: −0.57% | |
| (placebo- | 14 mg: −1.4% | | 10 mg: −0.73% | | | |
| adjusted | (trial product | | | | | |
| change from | estimate) | | | | | |
| baseline) | | | | | | |
| Effect on | 3 mg: −0.2 kg | 100 mg: +0.9 kg | 2.5 mg: +0.2 kg | 50 mg: +0.6 kg | 12.5 mg: −0.27 kg | 5 mg: −0.17 kg |
| Body Weight | (−0.23%) | (+1.1%) | (+0.22%) | (+0.65%) | 25 mg: −0.4 kg | |
| (placebo- | 7 mg: −1.0 kg | 200 mg: +1.0 kg | 5 mg: +1.3 kg | 100 mg: +1.2 kg | | |
| adjusted | (−1.1%) | (+1.2%) | (+1.4%) | (+1.3%) | | |
| change from | 14 mg: −2.6 kg | | 10 mg: +1.3 kg | | | |
| baseline) | (−3.0%) | | (+1.5%) | | | |
| | (trial product | | | | | |
| | estimand) | | | | | |
| Effects on | TC level change | Compared with | No significant | TG level change | TC (mg/dL): −1.2 | Serum total |
| Lipid | from baseline: −3% | controls, | effects on lipid | from baseline: +1% | (12.5 mg) ^{$$} | cholesterol and |
| Biomarkers | (3 mg) ^{$$} −5% | sitagliptin | biomarkers | (50 mg) ^{$} and +5% | and −3.9 | LDL cholesterol |
| | (7 mg) ^{$$$}, | alone or in | (Meta-analysis | (100 mg) vs. +19% | (25 mg) ^{$$$} | can be |
| | and −5% | combination | results) | (placebo) | vs. +10.1 | improved, |
| | (14 mg) ^{$$$} | significantly | | Other lipid | (placebo) | especially in |
| | vs. +2% | improved | | parameters | HDL (mg/dL): +0.9 | patients whose |
| | (placebo) | serum TG | | changed by <3% | (12.5 mg) | HbA1c level |
| | HDL level change | (weighted | | in all | and +0.4 | was decreased |
| | from baseline: 0% | mean | | treatment | (25 mg) | with linagliptin |
| | (3 mg), −2% | difference | | groups, and no | vs. +1.3 | treatment |
| | (7 mg), | [WMD] −0.24 | | significant | (placebo) | (Results of a |
| | and −1% (14 mg) | mmol/L) ^{$$} and | | between- | LDL (mg/dL): −0.5 | small |
| | vs. +1% | HDL-C (WMD | | treatment | (12.5 mg) | population |
| | (placebo) | 0.05 mmol/L) ^{$$$} | | differences | and −0.4 | single-arm |
| | LDL level change | However, | | were observed | (25 mg) | clinical trial) |
| | from baseline: −4% | no statistical | | | vs. +4.6 | |
| | (3 mg) ^{$}, −6% | significances | | | (placebo) | |
| | (7 mg) ^{$$}, | were observed | | | TG (mg/dL): −5.8 | |
| | and −5% (14 mg) ^{$$} | in LDL-C and | | | (12.5 mg) | |
| | vs. +2% | TC | | | and −17.8 | |
| | (placebo) | (Meta-analysis | | | (25 mg) ^{$} | |
| | TG level change | results) | | | vs. +26.5 | |
| | from baseline: −5% | | | | (placebo) | |
| | (3 mg), −9% | | | | | |
| | (7 mg) ^{$}, | | | | | |
| | and −9% (14 mg) ^{$} | | | | | |
| | vs. −2% | | | | | |
| | (placebo) | | | | | |
| | (26−week trial | | | | | |
| | product estimate | | | | | |
| | from another | | | | | |
| | clinical trial) | | | | | |

^{$}, ^{$$}, and ^{$$$} represent p < 0.05, <0.01, and <0.001 vs. placebo, respectively.

In view of similar results achieved by oral administration of a denatonium salt, having a different mechanism of action, a combination treatment with any of the GLP-IRA oral analog or a DPP-4 inhibitor is effective in combination for treating diabetes/MetS/glycemic control, weight loss/obesity and hyperlipidemia.

### GIP Analog

Another combination is GIP and GLP-1 co-analog combinations. Incretins are a group of metabolic hormones released in the gut that stimulate a decrease in blood glucose levels in a glucose-dependent manner. Incretins include the peptide hormones GLP-1 and GIP. Incretin hormones are released in enteroendocrine cells after eating. Both are dual incretin peptide mimetic compounds that agonize receptors for both human GIP and GLP-1.

### GLP-2 Analog

There is one approved GLP-2 analog, teduglutide (Gattex®). It is a 33 amino acid glucagon-like peptide-2 analog made in E. coli by a recombinant process (without glycosylation). It is injected sc (0.05 mg/kg) and indicated for short bowel syndrome. It has a half-life of 0.7 to 1.3 hr and many side effects, including fluid retention (1% to 12%); gastrointestinal reactions (12%-30%); antibody development (3% to 54%; incidence increased with prolonged use); injection site reaction (13%); upper respiratory tract infection (21%); intestinal stoma complication (42%).

In addition, eleven other GLP-2 analogs have been identified in various stages of clinical or pre-clinical development for short bowel syndrome or chemotherapy-induced diarrhea. These agents are listed in Table 4:

TABLE 4

| Drug name | Company | Modality | Mech of action |
|---|---|---|---|
| Glepaglutide | Zealand Pharma AS | Synthetic peptide for | GLP-2 receptor |
| (ZP-1848) | | s.c. | agonist |
| | | | (GLP-2 analog) |
| Apraglutide | VectivBio Holding | Synthetic peptide for | GLP-2 receptor |
| | AG | s.c. or i.v. | agonist |
| | | | (GLP-2 analog) |
| GXG-8 | Tasly | Fusion protein | GLP-2 receptor |
| | Pharmaceutical | | agonist |
| | Group | | |
| Elsiglutide | Zealand Pharma AS | Synthetic peptide for | GLP-2 receptor |
| (ZP-1846) | | s.c. | agonist |
| | | | (GLP-2 analog) |
| HM-15912 | Hanmi | Recombinant protein | GLP-2 receptor |
| | Pharmaceuticals | for s.c. | agonist |
| | | | (GLP-2 analog) |
| ZP-7570 | Zealand Pharma AS | Synthetic peptide for | Dual agonist of |
| | | s.c. | GLP-1 and GLP-2 |
| | | | recepors |
| GLP-2-ELP | PhaseBio | Fusion protein for | GLP-2 receptor |
| | Pharmaceuticals Inc | s.c. | agonist |
| Peptide to agonize | Sosei Heptares | Peptide | GLP-2 receptor |
| GLP-2 receptor | | | agonist |
| NB-1002 | 9 Meters Biopharma | Recombinant | GLP-2 receptor |
| | Inc | peptide | agonist |
| MOD-1501 | OPKO Health Inc | Fusion protein for | GLP-2 receptor |
| | | P.O. | agonist |
| HL-06 | Huons Global Co | Peptide | GLP-2 receptor |
| | Ltd | | agonist |

### PYY Analog

PYY is released during a meal from L-cells in the distal small intestine and the colon. PYY is known to have peripheral effects in the gastrointestinal (GI) tract. PYY is naturally secreted as a 36 amino acid peptide (PYY (1-36)) with a C-terminal amide but is cleaved to PYY (3-36) which constitutes approximately 50% of the circulating PYY. The enzyme responsible for the degradation is dipeptidyl peptidase IV (DPP-4). PYY (3-36) is rapidly eliminated by proteases and other clearance mechanisms. The half-life of PYY (3-36) has been reported to be <30 minutes in pigs (Ito T et al, Journal of Endocrinology (2006), 191, pp113-119). Thus, PYY displays suboptimal pharmacokinetic properties, meaning that the peptide must be administered at least twice daily and perhaps once daily together with a DPP-4 inhibitor.

Whereas PYY (1-36) activates Y1, Y2, and Y5 receptors with very little selectivity and the Y4 receptor slightly less, the DPP-4 processed PYY (3-36) displays increased selectivity for the Y2 receptor over Y1, Y4 and Y5 receptors, albeit some Y1 and Y5 affinity is retained. Y2 receptor activation decreases appetite and food intake whereas Y1 and Y5 receptor activation leads to an increase in appetite and food intake. Furthermore, Y1 and Y5 receptor activation may lead to an increase in blood pressure.

Based on demonstrated effects in e.g. Zucker rats and diet induced obese (DIO) mice, Y2 selective PYY (3-36) analogs demonstrated a positive effect on glucose (van den Hoek A. et al., Am. J. Physiol. Endocrinol. Meta. (2006), 292, ppE238-E245; and Ortiz A. et al, The Journal of Pharmacology and Experimental Therapeutics (2007), 323, pp 692-700). WO 2009/138511, WO 2011/033068 and WO 2011/058165 disclose long-acting Y2 and/or Y4 receptor agonists, PYY analogs stabilized against C-terminal proteolytic breakdown, and Y2 receptor agonists with protracted pharmacokinetic properties, respectively.

There are three PYY analogs that were found under development including NN-9775 (Novo-Nordisk) which is a synthetic peptide PYY analog that activates hypothalamic NPY-Y2 autoreceptors in phase 1 clinical trials for obesity; JNJ-0321 (J&J) a synthetic peptide as a long-acting PYY analog for obesity in preclinical development; and a Zihipp, Ltd. PYY analog for obesity that is in a very early stage. Like the GLP-2 or GLP-2 analogs, the PYY analogs also have challenges to preserve function at target receptors, increase immunogenicity (antibody formation), and increase potential risk for adverse effects via long-acting signaling, which is not reflective of normal physiology. Therefore, despite multiple such gut hormones analog development that almost all require injection (except an oral GLP-1 analog Rybelsus® that has lipid excipients for daily oral administration but requiring much higher doses) such biologic peptide gut hormone analogs are not amenable to oral delivery.

### CCK Analog

CCK is also a gut secreted peptide hormone that has appetite suppression properties. However, unlike PYY with a half-life of 9-14 min, the CCK half-life is 2-3 min. Developmental CCK analogs include C-2816 (Astra-Zeneca) which is a fusion peptide of GLP-IR agonist AC3174 plus CCKR1 agonist AC17022 for both receptors; NN-9056 (Novo-Nordisk) a synthetic peptide CCK analog for obesity; a Univ of Nebraska CCKR8 analog synthetic peptide; and A-71378 (AbbVie) CCK-8 analog synthetic peptide for obesity that appears to have been discontinued.

### Symphony of Gut Peptide Hormones (GPH)

Each of the gut peptide hormones (GLP-1, GLP-2, GIP, PYY, and CCK) described have various analogs either marketed or in development individually for glycemic control/diabetes and weight loss/obesity. Further DPP-4 is an enzyme that breaks down GLP-1 and PYY with several orally-active enzyme inhibitors available. The present disclosure provides a conductor for this symphony of multiple gut peptide hormones, a bitter receptor agonist, that is also able to play in all sections of the orchestra of gut peptide hormones. There is a need to treat glycemic control/diabetes, weight loss/obesity and hyperlipidemia by addressing multiple gut peptide hormones and not a single gut peptide hormone because gut signaling is driven by multiple gut peptide hormones and not only one gut peptide hormone. Therefore, the presently disclosed combination enhances single gut peptide hormone treatments by providing agonist activity for multiple gut peptide hormones by the addition of a denatonium salt component of a combination in view of the surprising data provided herein showing multiple relevant gut peptide hormone increases in whole animals and in a phase 1 human clinical trial.

### Pharmaceutical Compositions and Pharmaceutically Acceptable Carriers

Pharmaceutical compositions described herein and/or for use in the methods described herein may further comprise a pharmaceutically acceptable carrier. In some embodiments, the pharmaceutically acceptable carrier means a pharmaceutically-acceptable substrate, material, composition or vehicle to aid in the process of delivery of the API to the patient, and/or to stabilize the API during transport for delivery to the patient, such as a diluent, solid filler, excipient, or manufacturing aid (e.g., lubricant, talc, magnesium, calcium or zinc stearate, or steric acid). The term “acceptable” as used in this sense means that the material is compatible with the other ingredients of the formulation and does not produce intolerable side effects injurious to the patient.

In solid pharmaceutical dosage forms of the invention for oral administration as disclosed herein (capsules, tablets, pills, powders, granules, and the like), the API may be mixed with a pharmaceutically-acceptable carrier including one or more pharmaceutically-acceptable excipients such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

### Further Alternative Embodiments

The present disclosure provides the following embodiment (“Method 1”) of the method for treating or preventing progression of a fatty liver disease comprising administering to a subject having the fatty liver disease a combination of a denatonium salt, wherein the denatonium salt is selected from the group consisting of denatonium acetate (DA), denatonium citrate, denatonium maleate, denatonium saccharide, and denatonium tartrate; and a GLP-1 receptor agonist (e.g., semaglutide, glyburide, liraglutide, dulaglutide, albiglutide, exenatide, or lixisenatide). In some embodiments, the method further comprises administering from about 0.5 g to about 5 g acetic acid. Preferably, the dosage per day of the acetic acid for an adult is from about 1.5 g to about 3 g.
1.1 Method 1, wherein the fatty liver disease is NASH.
1.2 Method 1 or 1.1, wherein the fatty liver disease is NASH or NAFLD or ASH.
1.3 Any of the preceding Methods, wherein the denatonium salt is denatonium acetate.
1.4 Any of the preceding Methods, wherein the denatonium salt is denatonium citrate.
1.5 Any of the preceding Methods, wherein the denatonium salt is denatonium maleate.
1.6 Any of the preceding Methods, wherein the daily dose of the denatonium salt is 200 mg administered QD or BID.
1.7 Any of the preceding Methods, wherein the daily dose of the denatonium salt is 400 mg administered BID.
1.8 Any of the preceding Methods, wherein the daily dose of the denatonium salt is 600 mg administered QD or BID.
1.9 Any of the preceding Methods, wherein the daily dose of the denatonium salt is 1000 mg administered BID.
1.10 Any of the preceding Methods, wherein the GLP-1 receptor agonist is semaglutide.
1.11 Any of Methods 1.1-1.10, wherein the GLP-1 receptor agonist is glyburide.
1.12 Any of Methods 1.1-1.10, wherein the GLP-1 receptor agonist is liraglutide
1.13 Any of Methods 1.1-1.10, wherein the GLP-1 receptor agonist is dulaglutide.
1.14 Any of Methods 1.1-1.10, wherein the GLP-1 receptor agonist is albiglutide.
1.15 Any of Methods 1.1-1.10, wherein the GLP-1 receptor agonist is exenatide.
1.16 Any of Methods 1.1-1.10, wherein the GLP-1 receptor agonist is or lixisenatide.

In an alternative embodiment, the present disclosure provides Method 2, comprising a method for treating obesity and/or effecting weight loss, by administering an effective amount of an orally administered pharmaceutical composition in a single dosage form or in two dosage forms, wherein the pharmaceutical composition comprises a bitter receptor agonist and a gut signaling compound. Preferably in Method 2, the bitter receptor agonist is a denatonium salt, wherein the denatonium salt is selected from the group consisting of DA, denatonium citrate, denatonium maleate, denatonium saccharide, and denatonium tartrate; and the gut signaling compound is (a) a gut peptide hormone analog selected from the group consisting of a GLP-1RA, a GLP-2 analog, a PYY analog, a GIP analog, a CCK analog, and combinations thereof; or (b) a DPP-4 inhibitor selected from the group consisting of a salt of a medium chain fatty acid, a salt of N-(8-(2-hydroxybenzoyl)amino)caprylic acid, sodium N-(8-(2-hydroxybenzoyl)amino)caprylate (SNAC), sitagliptin phosphate, saxagliptin, linagliptin, and alogliptin.

Further, alternative embodiments of Method 2 include:
2.1 Method 2, wherein the GLP-1 analog is semaglutide, glyburide, liraglutide, dulaglutide, or albiglutide.
2.2 Method 2, wherein the PYY 1875 analog is NN-9775 or JNJ-9321.
2.3 Method 2, wherein the CCK analog is C-2819, NN-9056, or A-71378.
2.4 Method 2, wherein the GLP-2 analog is teduglutide, glepaglutide, apraglutide, elsiglutide, HM-15912, ZP-7570 GLP-2-ELP MOD-1501, or HL-06.
2.5 Method 2, wherein the gut signaling compound is a DPP-4 inhibitor selected from the group consisting of sodium N-(8-(2-hydroxybenzoyl)amino)caprylate (SNAC), sitagliptin phosphate, saxagliptin, linagliptin, and alogliptin, administered at a daily dose that is an approved daily dose for the specific DPP-4 inhibitor.
2.6 Method 2.5, wherein the DPP-4 inhibitor is administered once per day (QD) or twice per day about 8 hours apart (BID) and the denatonium salt is administered one per day or twice per day at a total daily dose (per weight of denatonium) of from about 50 mg to about 3000 mg, preferably from about 100 mg to about 2000 mg, and most preferably from about 200 mg to about 1000 mg.

In an alternative embodiment, there is provided Method 3 comprising a method for treating glycemic control, metabolic syndrome (MetS), and/or diabetes by administering an effective amount of an orally administered pharmaceutical composition in a single dosage form or two dosage forms, comprising a combination of a bitter receptor agonist comprising a denatonium salt, wherein the denatonium salt is selected from the group consisting of denatonium acetate (DA), denatonium citrate, denatonium maleate, denatonium saccharide, and denatonium tartrate; and either (a) a gut peptide hormone analog selected from the group consisting of a GLP-1RA, a GLP-2 analog, a PYY analog, a GIP analog, a CCK analog, and combinations thereof; or (b) a DPP-4 inhibitor selected from the group consisting of a salt of a medium chain fatty acid, a salt of N-(8-(2-hydroxybenzoyl)amino)caprylic acid, sodium N-(8-(2-hydroxybenzoyl)amino)caprylate (SNAC), sitagliptin phosphate, saxagliptin, linagliptin, and alogliptin.

Further embodiments of Method 3 include:
3.1 Method 3, wherein the method further comprises administering a DPP-4 inhibitor selected from a salt of a medium chain fatty acid, a salt of N-(8-(2-hydroxybenzoyl)amino)caprylic acid, sodium N-(8-(2-hydroxybenzoyl)amino)caprylate (SNAC), sitagliptin, saxagliptin, linagliptin, and alogliptin.
3.2 Method 3, wherein the GLP-1 analog is semaglutide, glyburide, liraglutide, dulaglutide, or albiglutide.
3.3 Method 3, wherein the PYY 1875 analog is NN-9775 or JNJ-9321.
3.4 Method 3, wherein the DPP-4 inhibitor is selected from sitagliptin phosphate, vildagliptin, linagliptin, alogliptin, saxagliptin, P93/01, SYR322, GSK 823093, Roche 0730699, TS021, E3024, and PHX-1149.
3.5 Method 3, wherein the GLP-2 analog is teduglutide, glepaglutide, apraglutide, elsiglutide, HM-15912, ZP-7570, GLP-2-ELP, MOD-1501, or HL-06.
3.6 Method 3, wherein the orally administered pharmaceutical composition is administered at a dosage for a commercially approved DPP-4 inhibitor selected from the group consisting of sodium N-(8-(2-hydroxybenzoyl)amino)caprylate (SNAC), sitagliptin phosphate, saxagliptin, linagliptin, and alogliptin, and is at a daily dose that is an approved daily dose for the specific DPP-4 inhibitor that is administered once per day (QD) or twice per day about 8 hours apart (BID); and the denatonium salt is administered one per day or twice per day at a total daily dose (per weight of denatonium) of from about 50 mg to about 3000 mg, preferably from about 100 mg to about 2000 mg, and most preferably from about 200 mg to about 1000 mg.

In another embodiment, there is provided Method 4, which is a method for treating hyperlipidemia comprising administering an effective amount of an orally administered pharmaceutical composition in a single dosage form or in two dosage forms, comprising a bitter receptor agonist comprising a denatonium salt, wherein the denatonium salt is selected from the group consisting of denatonium acetate (DA), denatonium citrate, denatonium maleate, denatonium saccharide, and denatonium tartrate; and either (a) gut peptide hormone analog selected from the group consisting of a glucagon-like peptide (GLP-1) analog, a GLP-2 analog, a PYY analog, a GIP analog, a CCK analog, and combinations thereof; or (b) a DPP-4 inhibitor selected from the group consisting of a salt of a medium chain fatty acid, a salt of N-(8-(2-hydroxybenzoyl)amino)caprylic acid, sodium N-(8-(2-hydroxybenzoyl)amino)caprylate (SNAC), sitagliptin phosphate, saxagliptin, linagliptin, and alogliptin.

Further embodiments of this Method 4 include:
4.1 Method 4, wherein the method further comprises administering a DPP-4 inhibitor selected from a salt of a medium chain fatty acid, a salt of N-(8-(2-hydroxybenzoyl)amino)caprylic acid, sodium N-(8-(2-hydroxybenzoyl)amino)caprylate (SNAC), sitagliptin, saxagliptin, linagliptin, and alogliptin.
4.2 Method 4, wherein the GLP-1 analog is semaglutide, glyburide, liraglutide, dulaglutide, or albiglutide.
4.3 Method 4, wherein the PYY 1875 analog is NN-9775 or JNJ-9321.
4.4 Method 4, wherein the DPP-4 inhibitor is selected from sitagliptin phosphate, vildagliptin, linagliptin, alogliptin, saxagliptin, P93/01, SYR322, GSK 823093, Roche 0730699, TS021, E3024, and PHX-1149.
4.5 Method 4, wherein the GLP-2 analog is teduglutide, glepaglutide, apraglutide, elsiglutide, HM-15912, ZP-7570, GLP-2-ELP, MOD-1501, or HL-06.
4.6 Method 4, wherein the orally administered pharmaceutical composition is administered at a dosage for a commercially approved DPP-4 inhibitor selected from the group consisting of sodium N-(8-(2-hydroxybenzoyl)amino)caprylate (SNAC), sitagliptin phosphate, saxagliptin, linagliptin, and alogliptin, at a daily dose that is an approved daily dose for the specific DPP-4 inhibitor that is administered once per day (QD) or twice per day about 8 hours apart (BID and the denatonium salt is administered one per day or twice per day at a total daily dose (per weight of denatonium) of from about 50 mg to about 3000 mg, preferably from about 100 mg to about 2000 mg, and most preferably from about 200 mg to about 1000 mg.

In another embodiment, there is provided Method 5, which is a method for lowering a dose administered of a GLP-1RA drug comprising co-administering with the GLP-1RA drug, a bitter receptor agonist comprising a denatonium salt, wherein the denatonium salt is selected from the group consisting of denatonium acetate (DA), denatonium citrate, denatonium maleate, denatonium saccharide, and denatonium tartrate.

Further embodiments of Method 5 include:
5.1 Method 5, wherein the GLP-1RA is semaglutide, glyburide, liraglutide, dulaglutide, or albiglutide.
5.2 Method 5, wherein the denatonium salt is DA.

Alternatively, the present disclosure further provides combination products. For example, according to one embodiment of the invention (Combination 1), there is provided an oral dosage form pharmaceutical composition comprising a bitter receptor agonist and a dipeptidyl peptidase-4 (DPP-4) inhibitor.

Further embodiments of Combination 1 include:
1.1 Combination 1, wherein the bitter receptor agonist is selected from the group consisting of denatonium salts, including acetate (DA), denatonium citrate, denatonium maleate, denatonium saccharide, and denatonium tartrate, chlorpheniramine, diphenidol, famotidine, haloperidol, quinine, parthenolide, and aristolochic acid.
1.2 Combination 1, wherein the DPP-4 inhibitor is selected from the group consisting of a salt of a medium chain fatty acid, a salt of N-(8-(2-hydroxybenzoyl)amino)caprylic acid, sodium N-(8-(2-hydroxybenzoyl)amino)caprylate (SNAC), sitagliptin, saxagliptin, linagliptin, alogliptin, and combinations thereof.

Alternatively, according to another embodiment (Combination 2), there is provided a synergistic combination pharmaceutical composition comprising a formulation of a bitter receptor agonist and a gut signaling peptide analog and gut signaling hormone enhancers (oral formulation) selected from the group consisting of glucagon-like peptide (GLP-1) analogs, peptide YY (PYY) analogs, dipeptidyl peptidase-4 (DPP-4) inhibitors, and glucose-dependent insulinotropic polypeptide (GIP) analogs.

Further embodiments of Combination 2 include:
2.1 Combination 2, wherein the pharmaceutical combination further comprises a DPP-4 inhibitor.
2.2 Combination 2, wherein the bitter receptor agonist is selected from the group consisting of denatonium salts, including acetate (DA), denatonium citrate, denatonium maleate, denatonium saccharide, and denatonium tartrate, chlorpheniramine, diphenidol, famotidine, haloperidol, quinine, parthenolide, and aristolochic acid.
2.3 Combination 2, wherein, the DPP-4 inhibitor is selected from the group consisting of a salt of a medium chain fatty acid, a salt of N-(8-(2-hydroxybenzoyl)amino)caprylic acid, sodium N-(8-(2-hydroxybenzoyl)amino)caprylate (SNAC), sitagliptin, saxagliptin, linagliptin, alogliptin, and combinations thereof.

According to another embodiment of the invention (Combination 3), there is provided a combination pharmaceutical composition for oral administration comprising a formulation of a bitter receptor agonist comprising a denatonium salt, wherein the denatonium salt is selected from the group consisting of denatonium acetate (DA), denatonium citrate, denatonium maleate, denatonium saccharide, and denatonium tartrate; and a DPP-4 inhibitor, selected from the group consisting of a salt of a medium chain fatty acid, a salt of N-(8-(2-hydroxybenzoyl)amino)caprylic acid, sodium N-(8-(2-hydroxybenzoyl)amino)caprylate (SNAC), sitagliptin salts, saxagliptin, linagliptin, alogliptin, and combinations thereof.

Further embodiments of Combination 3 include:
3.1 Combination 3, wherein the denatonium salt is denatonium acetate and the DPP-4 inhibitor is sitagliptin in a single oral dosage form.
3.2 Combination 3, further comprising an oral dosage form of a GLP-1RA semaglutide.
3.3 Combination 3, wherein a dosage for a commercially approved DPP-4 inhibitor selected from the group consisting of sodium N-(8-(2-hydroxybenzoyl)amino)caprylate (SNAC), sitagliptin phosphate, saxagliptin, linagliptin, and alogliptin is at a daily dose that is an approved daily dose for the specific DPP-4 inhibitor that is administered once per day (QD) or twice per day about 8 hours apart (BID), and the denatonium salt is administered one per day or twice per day at a total daily dose (per weight of denatonium) of from about 50 mg to about 3000 mg, preferably from about 100 mg to about 2000 mg, and most preferably from about 200 mg to about 1000 mg. In one embodiment, the dosage is 1000 mg.

It is contemplated that for each of the Methods and Combinations recited herein (e.g., Methods 1-5 and Combinations 1-3, above, and each subpart thereof), alternative embodiments and dosages of the denatonium salt may be used and administered to a human patient. For example, in some embodiments, the dosage of denatonium salt is from about 0.5 mg/kg BID to about 30 mg/kg BID, or optionally, from about 1 mg/kg BID to about 20 mg/kg BID. In alternative embodiments, the dosage of denatonium salt administered to a human patient is from about 1.0 mg/kg/day to about 60 mg/kg/day; in some embodiments, from about 2 mg/kg/day to about 40 mg/kg/day; and optionally, from about 4 mg/kg/day to about 20 mg/kg/day. In another embodiment, the dosage range of denatonium salt used in the inventive Methods and Combinations is from about 0.1 mg/kg/day to about 32 mg/kg/day, preferably from about 0.25 mg/kg/day to about 16 mg/kg/day; and most preferably from about 0.5 mg/kg/day to about 8 mg/kg/day.

### EXAMPLES

Reference will now be made in detail to certain embodiments illustrated in the following Examples and accompanying drawings. While the disclosure provides exemplary embodiments, it will be understood that the examples are not intended to limit the disclosure to those embodiments. On the contrary, the invention is intended to cover all alternatives, modifications, and equivalents which may be appreciated by one skilled in the field from the disclosures.

### Example 1a

### Preparation of DA, Bitter Receptor Agonist

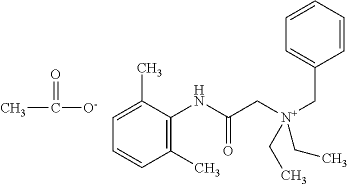

Denatonium acetate anhydrous, or DA is an anhydrous salt such that for every 100 mg of DA, there are 83 mg of denatonium cation, 17 mg of acetate anion.

This Scheme A describes the synthesis of denatonium acetate (DA).

Step 1: Synthesis of Denatonium Hydroxide from Lidocaine

To a reflux apparatus add 25 g of lidocaine, 60 ml of water and 17.5 g of benzyl chloride with stirring and heating in 70-90° C. The solution needs to be heated and stirred in the before given value for 24 h, the solution needs to be cooled down to 30° C. The unreacted reagents are removed with 3×10 mL of toluene. With stirring dissolve 65 g of sodium hydroxide into 65 mL of cold water and add it to the aqueous solution with stirring over the course of 3 h. Filter the mixture, wash with some water and dry in open air. Recrystallize in hot chloroform or hot ethanol.

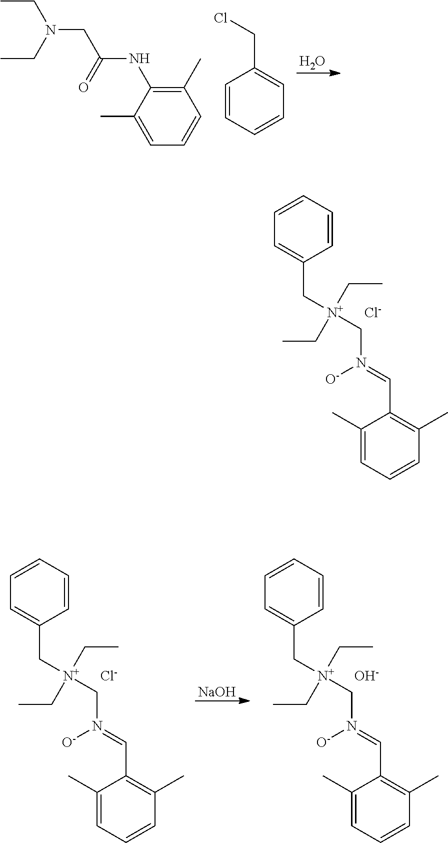

Step 2: Preparation of Denatonium Acetate Anhydrous from Denatonium Hydroxide.

To a reflux apparatus 10 g of denatonium hydroxide (MW: 342.475 g/mol, 0.029 mol), 20 mL of acetone, and 2 g of acetic acid glacial (0.033 mol) dissolved in 15 mL of acetone is added, the mixture is stirred and heated to 35° C. for 3 h. Then evaporated to dryness and recrystallized in hot acetone.

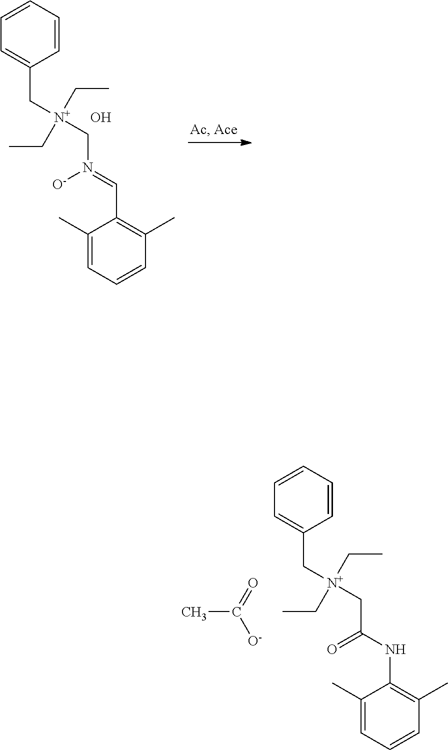

### Example 1b

### Preparation of Pharmaceutical Composition Comprising DA

Formulation of DA particles that can be formed into a tablet or filled in a capsule for oral delivery that avoids mouth cavity exposure.

This Example provides an immediate release 50 mg granule formulation of denatonium acetate (DA) as a free base as an immediate gastric release oral pharmaceutical formulation. Detailed manufacturing steps are described below.

### 1. Drug Layering Process—Drug Layered Pellets

Drug layering process was performed in a Fluid bed granulator equipped with the rotor insert (rotor granulator). Drug solution was prepared by solubilizing Povidone K30 (Kollidon 30) and Denatonium Acetate in ethyl alcohol. The drug solution was sprayed tangentially on to the bed of sugar spheres (35/45 mesh) moving in a circular motion in the rotor granulator. The final drug loaded pellets were then dried for ten (10) minutes in the rotor granulator, discharged and screened through a #20 mesh.

### 2. Seal Coating Process—Seal Coated Pellets

Seal coating dispersion was prepared by separately dissolving Hypromellose E5 in a mixture (1:1) of ethyl alcohol and purified water until a clear solution was obtained. The remaining quantity of ethyl alcohol was then added to the above solution followed by talc. The dispersion was mixed for 20 minutes to allow for uniform dispersion of talc. The seal coating dispersion was sprayed tangentially on to the drug loaded pellets to achieve 5% weight gain. The seal coated pellets were then dried for five (5) minutes in the rotor granulator, discharged and dried further in a tray dryer/oven at 55° C. for 2 hours. The seal coated pellets were then screened through a #20 mesh.

### 3. Final Blending—Denatonium Immediate Release (IR) Pellets

The seal coated pellets were blended with talc screened through mesh #60 using a V-Blender for ten (10) minutes and discharged. The blended seal coated beads, Denatonium IR Pellets, were used for encapsulation.

### 4. Encapsulation—Denatonium Capsules, 50 mg

The Denatonium IR pellets from step 3 (50 mg), were filled into size 1, white opaque hard gelatin capsules using an auto capsule filling machine. Capsules were then passed through an in-line capsule polisher and metal detector. In-process controls for capsule weight and appearance was performed during the encapsulation process. Acceptable quality limit (AQL) sampling and testing was performed by Quality Assurance (QA) on a composite sample during the encapsulation process. Finished product composite sample was collected and analyzed as per specification for release testing.

5. Packaging—Capsules, 50 mg—30 Counts

The 50 mg capsules were packaged in 30 counts into 50/60 cc White HDPE round S-line bottles with 33 mm White CRC Caps. The bottles were torqued and sealed using an induction sealer.

Table 5 shows qualitative and quantitative formulation composition of DA.

TABLE 5

| | | | | | Limits based on IID | |
|---|---|---|---|---|---|---|
| | | | | | Max Potency | |
| | Quality | | Quantity | DA capsule-50 | for Unit | |
| Ingredient | Standard | Function | (%) w/w | mg (mg/cap) | Dose (mg) | Reference |
| Denatonium | In-house | API | 23.55 | 59.03 (20 mg | N/A | N/A |
| acetate | | | | Denatonium | | |
| | | | | base) | | |
| Povidone | USP | Binder | 2.36 | 5.90 | 61.5 | Oral - |
| (KOLLIDON 30) | | | | | | Capsule |
| Sugar Spheres | NF | Substrate | 68.85 | 172.57 | 314.13 | Oral - |
| (VIVAPHARM ® | | | | | | Capsule |
| Sugar Spheres | | | | | | |
| 35-45) | | | | | | |
| Hypromellose | USP | Binder | 3.64 | 9.14 | 150 | Oral - |
| (Methocel E5 | | | | | | Capsule |
| Premium LV | | | | | | |
| Hydroxypropyl | | | | | | |
| Methylcellulose) | | | | | | |
| Talc | USP | Anti- | 1.09 | 2.74 | 14 | Oral - |
| (MicroTalc | | tacking | | | | Capsule, |
| MP 1538 USP | | agent | | | | coated |
| Talc) | | | | | | pellets |
| Talc (extra | USP | Flow aid | 0.50 | 1.25 | 284.38 | Oral - |
| granular) | | | | | | Capsule |
| (MicroTalc | | | | | | |
| MP 1538 USP | | | | | | |
| Talc) | | | | | | |
| Total weight of beads | | | 250.62 | | N/A | N/A |
| Hard Gelatin | USP | Capsule | N/A | 73.3 | 107 | Oral - |
| Capsule | | shell | | | | Capsule |
| Shells; | | | | | | |
| Cap: White | | | | | | |
| Opaque: | | | | | | |
| Body: White | | | | | | |
| Opaque; | | | | | | |
| Size: 1 | | | | | | |

| | | | |
|---|---|---|---|
| Total weight of Filled Capsule | 323.9 | N/A | N/A |

IID, the Inactive Ingredient Database;
API, active pharmaceutical ingredient;
USP, the US Pharmacopeia;
NF, the National Formulary
* Solvents such as Ethyl Alcohol USP 190 Proof (190 Proof Pure Ethyl Alcohol) and purified water (USP) were used for the preparation of drug solution and seal coating dispersion but are removed during the manufacturing process.

### Example 2

This example describes an in vivo study in a high fat diet induced obese (DIO) mouse model following treatment with denatonium acetate, liraglutide (GLP-1 agonist), or their combination for 4 weeks. This study included four treatment groups, with 15 mice assigned for each: (1) vehicle-treated group, orally (PO) administered with distilled water, twice-daily (BID) and subcutaneously (SC) administered with sterile 0.9% saline solution, BID; (2) denatonium acetate (DA)-treated group, PO administered with 75 mg/kg (denatonium salt weight) of DA, BID; (3) liraglutide-treated group, SC administered with 200 μg/kg of liraglutide, BID; and (4) the combination-treated group, PO administered with 75 mg/kg (denatonium salt weight) of DA, BID plus SC administered with 200 μg/kg of liraglutide, BID. The dosing regimen used is shown in Table 6.

TABLE 6
Dosing Regimen of the Study

| | | | Dose | | Dose | | | Dose | |
|---|---|---|---|---|---|---|---|---|---|
| Grp | N | Treatment | Level | | Concentration | | Dose Vol. | Route | Regimen |
| 1 | 15 | Vehicle | — | | — | | 5 mL/kg | PO | BID for both |
| | | (distilled | | | | | for PO and | (distilled | PO and SC. 4- |
| | | water + | | | | | 1 mL/kg | water) + | week dosing |
| | | sterile | | | | | for SC | SC | |
| | | saline | | | | | | (sterile | |
| | | solution) | | | | | | saline | |
| | | | | | | | | solution) | |
| 2 | 15 | DA | 75 | mg/kg | 15 | mg/mL | 5 mL/kg | PO | |
| 3 | 15 | Liraglutide | 200 | μg/kg | 200 | μg/mL | 1 mL/kg | SC | |

| | | | | | | |
|---|---|---|---|---|---|---|
| 4 | 15 | DA + | 75 mg/kg | 15 mg/mL | 5 mL/kg | PO for |
| | | Liraglutide | for DA + | for DA + | for DA and | DA and |
| | | | 200 μg/ | 200 μg/ | 1 mL/ | SC for |
| | | | kg for | mL for | kg for | liraglutide |
| | | | Liraglutide | Liraglutide | liraglutide | |

BID, twice daily.
DA, denatonium acetate.
SC, subcutaneous.
PO, by mouth (oral gavage).

After arrival at the testing facility, all animals were acclimated to the vivarium for a period no less than 3 days and placed on a 60% Fat Rodent Diet (D12492; Research Diets), 12:12 dark/light cycle and group housed, 2-3 in hepa-filtered cages. All treatments were continued for four weeks. During the study period, gross observation (animal behavior and clinical signs) was conducted, and body weight measurement was performed three times per week for each animal. At the end of the study, all animals were fasted overnight before measurements were taken for serum levels of glucose, insulin, hemoglobin A1c (HbA1c), low-density lipoprotein (LDL) cholesterol, high-density lipoprotein (HDL) cholesterol, triglyceride (TG), and bile acid (BA). Statistical analysis of data was performed using one tail Student's t-test on Excel.

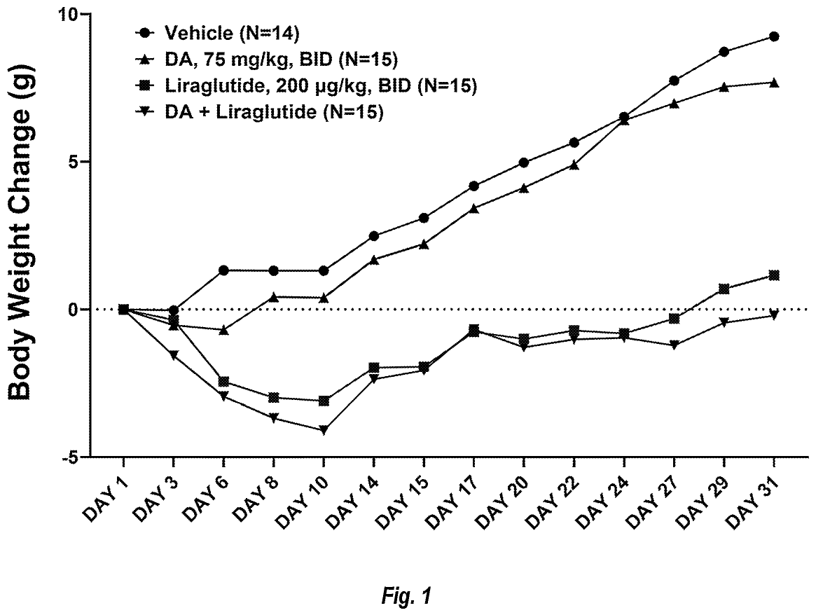

FIG. 1 shows the average body weight gain across the study period for all treatment groups. Table 7 shows body weight gain at the end of the study (Day 31) for each anima in all treatment groups. These data reveal that (1) body weight gain was significantly reduced in DIO mice upon treatment with DA, liraglutide, or their combination; and (2) the combination of DA and liraglutide produced a significantly lower body weight gain as compared to DA or liraglutide alone, indicating a potential synergistic (or at least additive) effect on body weight gain between the two agents.

TABLE 7
Body Weight gain at the end of the study (Day 31)

| | | Body | | | | |
|---|---|---|---|---|---|---|
| | Animal | Weight | | | | |
| Group | No. | Gain (g) | Mean (g) | SE (g) | p value ^{a} | p value ^{b} |
| Vehicle (distilled | 1.2 | 7.2 | 9.25 | 0.72 | NA | <0.0001 |
| water, PO, BID/ | 1.3 | 6.9 | | | | |
| sterile saline, | 1.4 | 9.1 | | | | |
| SC, BID) | 1.5 | 7.8 | | | | |
| | 1.6 | 8 | | | | |
| | 1.7 | 7.6 | | | | |
| | 1.8 | 7.3 | | | | |
| | 1.9 | 9.6 | | | | |
| | 1.10 | 16.6 | | | | |
| | 1.11 | 11.4 | | | | |
| | 1.12 | 10.5 | | | | |
| | 1.13 | 11 | | | | |
| | 1.14 | 6.1 | | | | |
| | 1.15 | 10.4 | | | | |
| DA 75 mg/kg (PO, | 2.1 | 10.1 | 7.69 | 0.46 | 0.04 | <0.0001 |
| BID) | 2.2 | 6.7 | | | | |
| | 2.3 | 7.2 | | | | |
| | 2.4 | 7.2 | | | | |
| | 2.5 | 9.6 | | | | |
| | 2.6 | 9.6 | | | | |
| | 2.7 | 7.4 | | | | |
| | 2.8 | 7.2 | | | | |
| | 2.9 | 5.2 | | | | |
| | 2.10 | 7 | | | | |
| | 2.11 | 8.1 | | | | |
| | 2.12 | 6.9 | | | | |
| | 2.13 | 11.2 | | | | |
| | 2.14 | 4.5 | | | | |
| | 2.15 | 7.5 | | | | |
| Liraglutide | 3.1 | 2 | 1.15 | 0.43 | <0.0001 | 0.013 |
| 200 μg/kg | 3.2 | −0.3 | | | | |
| (SC, BID) | 3.3 | 2.3 | | | | |
| | 3.4 | 1.3 | | | | |
| | 3.5 | 3.1 | | | | |
| | 3.6 | 2.7 | | | | |
| | 3.7 | 1.4 | | | | |
| | 3.8 | 1.8 | | | | |
| | 3.9 | 1.2 | | | | |
| | 3.10 | 2.1 | | | | |
| | 3.11 | 1.6 | | | | |
| | 3.12 | −2.9 | | | | |
| | 3.13 | 2.4 | | | | |
| | 3.14 | 0.1 | | | | |
| | 3.15 | −1.5 | | | | |
| DA 75 mg/kg (PO, | 4.1 | −0.2 | −0.21 | 0.39 | <0.0001 | NA |
| BID) Plus | 4.2 | 0.2 | | | | |
| liraglutide | 4.3 | 0.3 | | | | |
| 200 μg/kg | 4.4 | −1.5 | | | | |
| (SC, BID) | 4.5 | 1.3 | | | | |
| | 4.6 | −0.3 | | | | |
| | 4.7 | 1.1 | | | | |
| | 4.8 | −3.5 | | | | |
| | 4.9 | 0.7 | | | | |
| | 4.10 | 1.4 | | | | |
| | 4.11 | 0.2 | | | | |
| | 4.12 | −3.3 | | | | |
| | 4.13 | −0.7 | | | | |
| | 4.14 | 0.4 | | | | |
| | 4.15 | 0.8 | | | | |

BID, twice daily.
DA, denatonium acetate.
SC, subcutaneous.
PO, by mouth (oral gavage).
NA, not applicable.
^{a} p value by one-tailed non-paired t-test vs. Group 1 (vehicle). A difference was considered statistically significant with p < 0.05.
^{b} p value by one-tailed non-paired t-test vs. Group 4 (the combination of DA and liraglutide). A difference was considered statistically significant with p < 0.05.

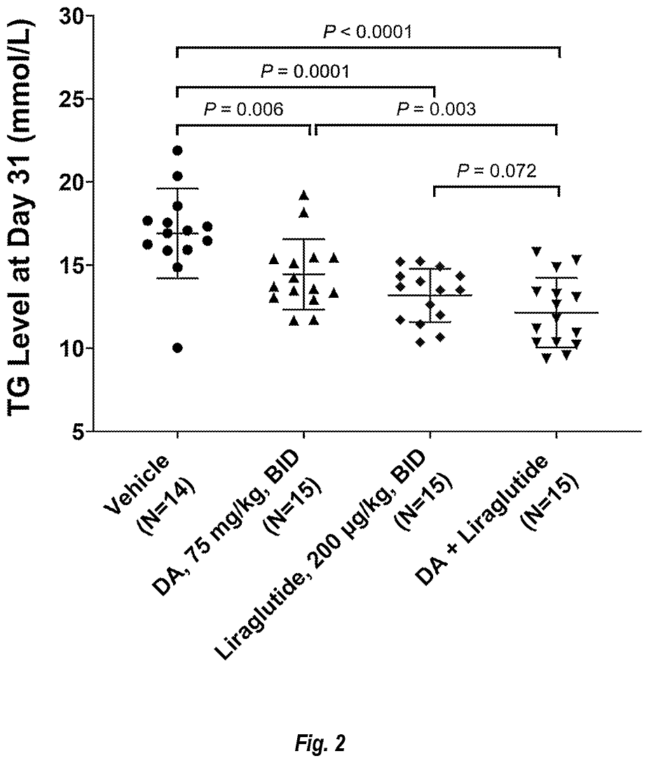

Table 8 and FIG. 2 present serum TG level at the end of study (Day 31) for each animal in the four treatment groups. These data show that (1) treatment with DA, liraglutide, or their combination significantly decreased serum TG level in DIO mice as compared to vehicle; and (2) animals treated the combination showed a significantly lower serum TG level as compared to those treated with DA or liraglutide alone, indicating a potential synergistic (or at least additive) effect on serum TG level between the two agents.

TABLE 8
Serum TG Level for Each Animal at the End of Study

| | | Serum TG | | | | |
|---|---|---|---|---|---|---|
| | Animal | Level | Mean | SE | | |
| Group | No. | (mmol/L) | (mmol/L) | (mmol/L) | p value ^{a} | p value ^{b} |
| Vehicle (distilled | 1.2 | 15.88 | 16.91 | 0.72 | NA | <0.0001 |
| water, PO, BID/ | 1.3 | 20.35 | | | | |
| sterile saline, | 1.4 | 17.67 | | | | |
| SC, BID) | 1.5 | 16.25 | | | | |
| | 1.6 | 10.04 | | | | |
| | 1.7 | 17.08 | | | | |
| | 1.8 | 16.93 | | | | |
| | 1.9 | 14.87 | | | | |
| | 1.10 | 16.47 | | | | |
| | 1.11 | 15.93 | | | | |
| | 1.12 | 17.55 | | | | |
| | 1.13 | 17.32 | | | | |
| | 1.14 | 18.55 | | | | |
| | 1.15 | 21.90 | | | | |
| DA 75 mg/kg (PO, | 2.1 | 13.06 | 14.45 | 0.55 | 0.006 | 0.003 |
| BID) | 2.2 | 13.49 | | | | |
| | 2.3 | 11.69 | | | | |
| | 2.4 | 13.35 | | | | |
| | 2.5 | 18.18 | | | | |
| | 2.6 | 14.27 | | | | |
| | 2.7 | 15.38 | | | | |
| | 2.8 | 12.93 | | | | |
| | 2.9 | 15.46 | | | | |
| | 2.10 | 19.24 | | | | |
| | 2.11 | 11.73 | | | | |
| | 2.12 | 15.13 | | | | |
| | 2.13 | 13.72 | | | | |
| | 2.14 | 13.59 | | | | |
| | 2.15 | 15.48 | | | | |
| Liraglutide | 3.1 | 11.46 | 13.18 | 0.41 | 0.0001 | 0.072 |
| 200 μg/kg | 3.2 | 13.50 | | | | |
| (SC, BID) | 3.3 | 14.35 | | | | |
| | 3.4 | 12.00 | | | | |
| | 3.5 | 14.92 | | | | |
| | 3.6 | 15.23 | | | | |
| | 3.7 | 15.20 | | | | |
| | 3.8 | 12.63 | | | | |
| | 3.9 | 13.70 | | | | |
| | 3.10 | 13.51 | | | | |
| | 3.11 | 11.71 | | | | |
| | 3.12 | 10.68 | | | | |
| | 3.13 | 14.02 | | | | |
| | 3.14 | 10.38 | | | | |
| | 3.15 | 14.34 | | | | |
| DA 75 mg/kg (PO, | 4.1 | 10.95 | 12.15 | 0.54 | <0.0001 | NA |
| BID) Plus | 4.2 | 11.79 | | | | |
| liraglutide | 4.3 | 13.27 | | | | |
| 200 μg/kg | 4.4 | 9.59 | | | | |
| (SC, BID) | 4.5 | 13.09 | | | | |
| | 4.6 | 10.37 | | | | |
| | 4.7 | 12.63 | | | | |
| | 4.8 | 15.79 | | | | |
| | 4.9 | 9.39 | | | | |
| | 4.10 | 15.31 | | | | |
| | 4.11 | 10.35 | | | | |
| | 4.12 | 11.20 | | | | |
| | 4.13 | 14.88 | | | | |
| | 4.14 | 10.22 | | | | |
| | 4.15 | 13.41 | | | | |

BID, twice daily.
DA, denatonium acetate.
SC, subcutaneous.
PO, by mouth (oral gavage).
NA, not applicable.
^{a} p value by one-tailed non-paired t-test vs. Group 1 (vehicle). A difference was considered statistically significant with p < 0.05.
^{b} p value by one-tailed non-paired t-test vs. Group 4 (the combination of DA and liraglutide). A difference was considered statistically significant with p < 0.05.

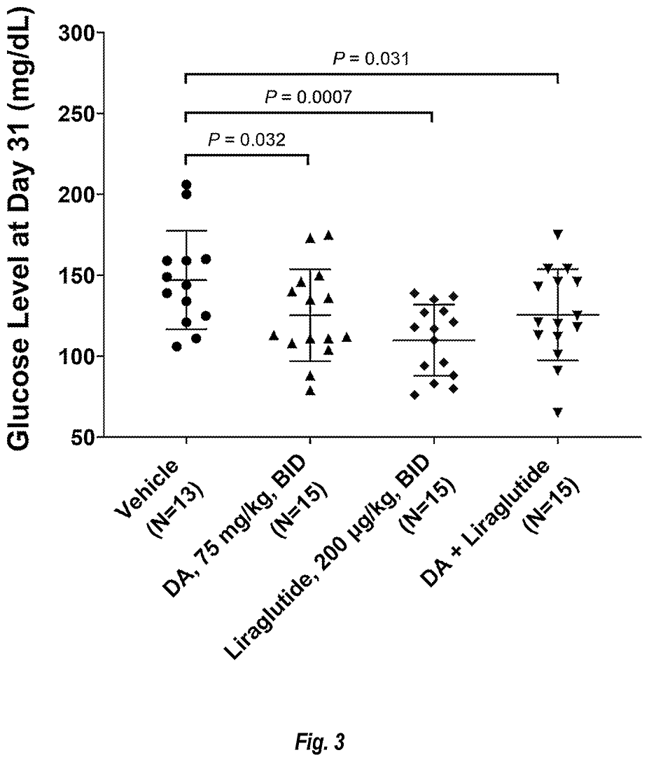

FIG. 3 presents serum glucose level at the end of study (Day 31) for each animal in the four treatment groups. Treatment with DA, liraglutide, or their combination significantly decreased serum glucose level in DIO mice upon 4-week dosing.

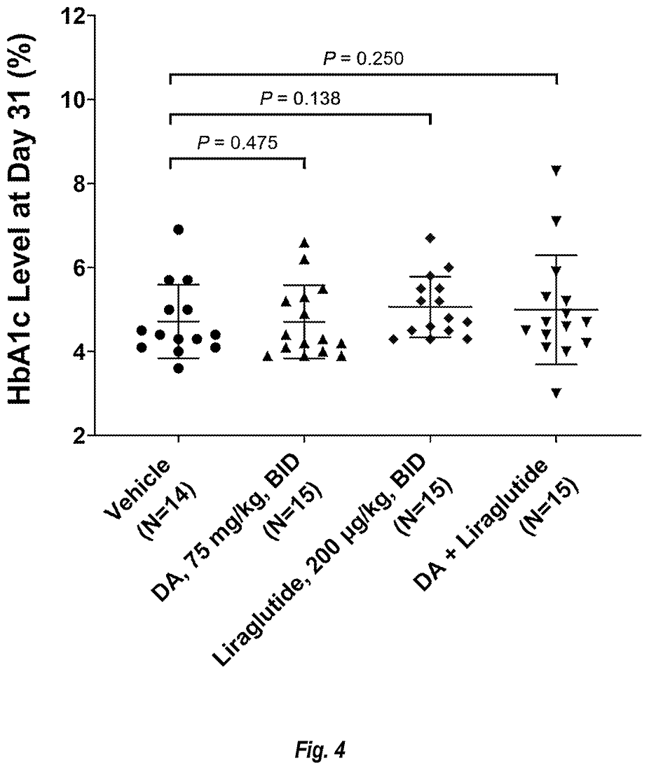

FIG. 4 shows serum HbA1c level at the end of study (Day 31) for each animal in the four treatment groups. The results suggest that treatment with DA, liraglutide, or their combination did not show significant effect (p>0.05) on serum HbA1c level in DIO mice after 4-week dosing.

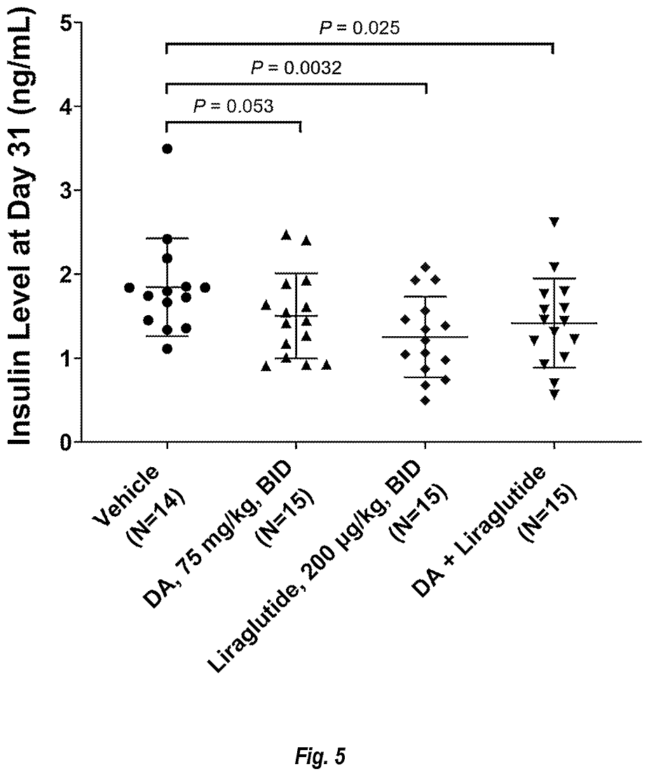

FIG. 5 depicts serum insulin level at the end of study (Day 31) for each animal in the four treatment groups. The data reveal that 4-week treatment with DA, liraglutide, or their combination considerably decreased serum insulin level in DIO mice.

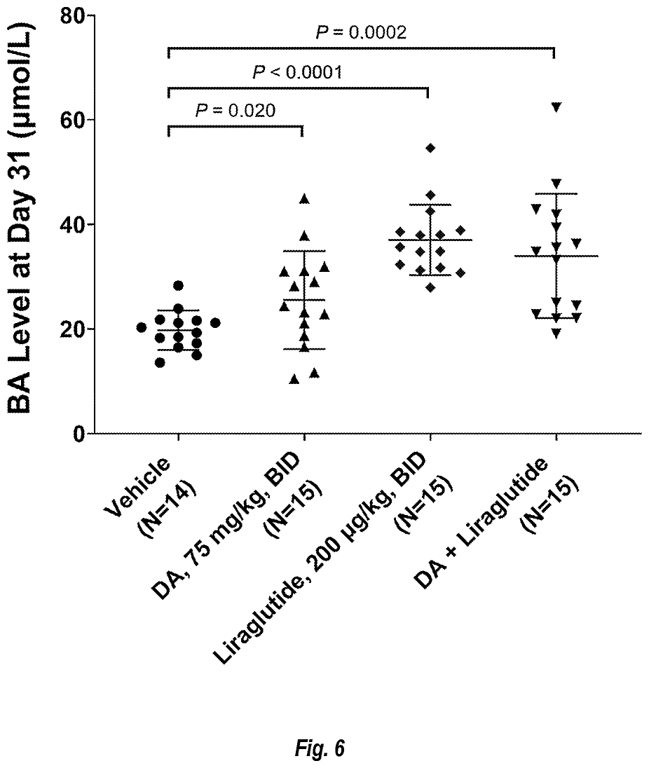

FIG. 6 presents serum BA level at the end of study (Day 31) for each animal in the four treatment groups. Upon 4-week dosing, DA, liraglutide, or their combination resulted in a significant increase in serum BA level as compared to vehicle control.

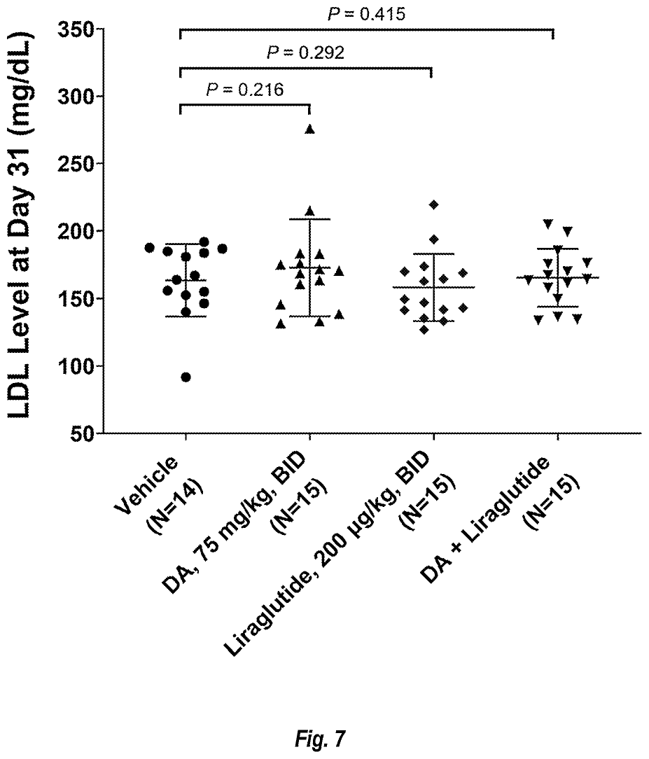

FIG. 7 shows serum LDL level at the end of study (Day 31) for each animal in the four treatment groups. There was no significant difference in serum LDL level among animals treated either with vehicle or with DA, liraglutide, or their combination.

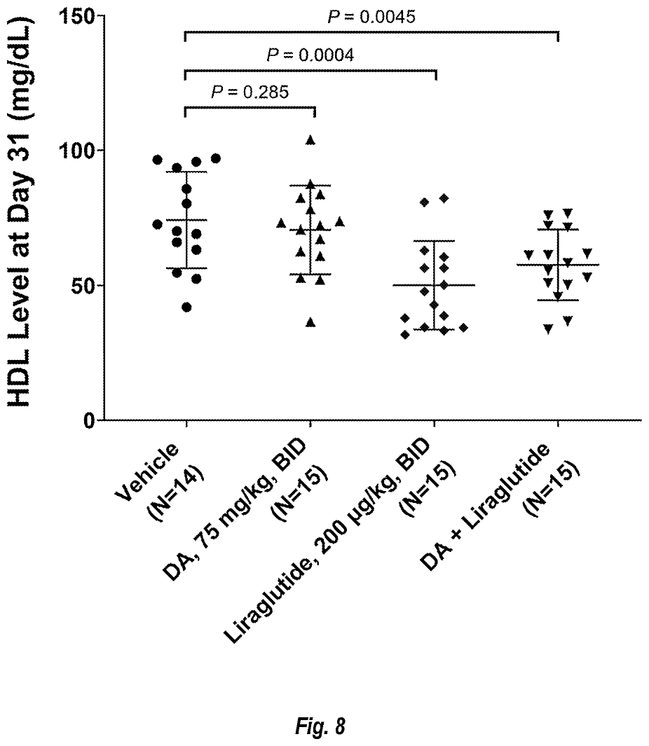

FIG. 8 shows serum HDL level at the end of study (Day 31) for each animal in the four treatment groups. The data reveal that as compared to vehicle, treatment with liraglutide, or the combination of DA plus liraglutide led to a significant decrease in serum HDL level in DIO mice after 4-week dosing.

### Example 3

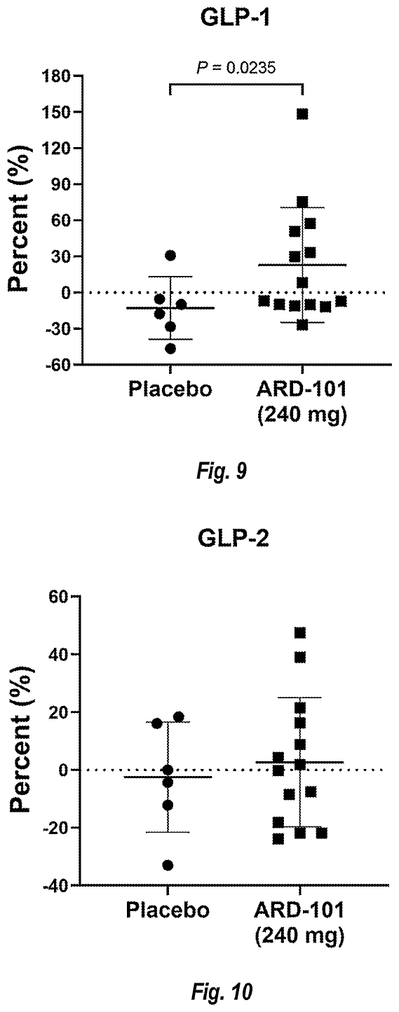

This Example provides data of three gut peptide hormones from a phase 1 clinical trial of denatonium acetate after a single dose of DA at 240 mg in a tablet in the formulation disclosed herein. The subjects were either placebo or 240 mg DA and blood samples were taken just prior to dosing and one hour after oral dosing. FIG. 9 shows the percent change for GLP-1 between time 0 and one hour after oral dosing. Even in a small sample size, the difference was significant p=0.0235. FIG. 10 shows the results for GLP-2, which showed a trend for GLP-2 gut peptide hormone increase. FIG. 11 shows the results for PYY, which showed a trend for PYY gut peptide hormone increase. Therefore, the data from this human clinical study showed that DA exerts its weight loss effect within the GI tract but stimulating release of the gut hormones GLP-1, GLP-2 and PYY.

### Example 4

This Example provides the results from an in vivo study of denatonium acetate and sitagliptin in high-fat diet-induced obese (DIO) mice. C57BL/6NTac mice (at least 12 weeks of age) were fed with a high fat diet. All mice were dosed orally by gavage as (a) Vehicle group (N=15) dosed with distilled water BID, (b) DA group (N=15) dosed at 75 mg/kg (denatonium weight) BID; (c) sitagliptin group (N=15) dosed by gavage with 10 mg/kg QD; and (d) DA+ sitagliptin group (N=15) treated with DA at 75 mg/kg (denatonium based weight) BID and sitagliptin at 10 mg/kg by gavage QD. The groups were dosed for 8 weeks followed by a 5-7 day sitagliptin period. Body weight and body weight changes were measured three times per week. Serum biomarker levels for blood glucose (after fasting 6-8 hours), blood insulin, percentage of blood HvA1c, HDL, LDL, total triglycerides (TG), total cholesterol (TC) and bile acid were measured twice during the study at day 28 and at the end (day 56) of the study. Cumulative food intake and water consumption for each animal was measured.

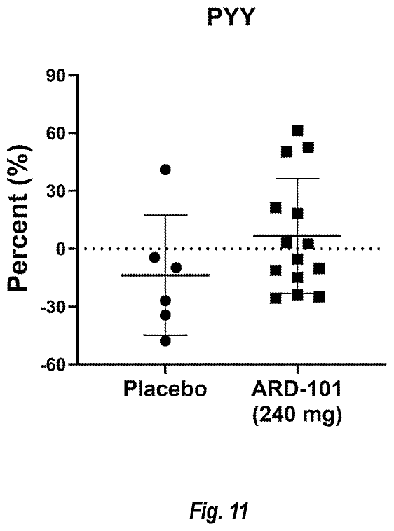

All 60 animals were well tolerated to the given treatments without significant toxic side effects observed during the study period. FIGS. 12A and 12B show relative body weight percentage (12A) and relative body weight change (g) (12B) for the four groups of animals. Treatment with sitagliptin alone showed the least effect on body weight, which is consistent with previous studies and clinical experience. However, much greater effect on body weight was seen with DA alone (ARD-101), but a considerable or synergistic effect on body weight was seen with the combination of DA and sitagliptin.

Accordingly, in Example 3 and the accompanying figures, DA (a denatonium salt with denatonium as the cation and acetate as the anion) showed superiority to sitagliptin (a representative of all of the other DPP-4i class drugs) head-to-head. There was an observed notable as well as statistically significant reduction of weight in the DA group versus the sitagliptin group. Additionally, unlike the “rebound” effect observed with sitagliptin over time (in Example 4 and consistent with clinical observations of the effect of DPP-4i drugs on obesity), DA continued to demonstrate continued weight loss and other parameter effects throughout the duration of the 56-day study (Example 3). It should be noted is that the combination of sitagliptin and DA together elicited an even more profound and statistically significant weight loss benefit compared to either agent alone. These data demonstrated profound synergism (the measured total effect is greater than the sum of each constituent agent in the combination). Further, there were no observed toxicity effects in the animals (Example 3 data) to indicate that the combination of both sitagliptin (and by extension other DPP-4i drugs) and DA would have notable cumulative toxicity effects of concern that would limit either concomitant dosing or a single combination dosage form.

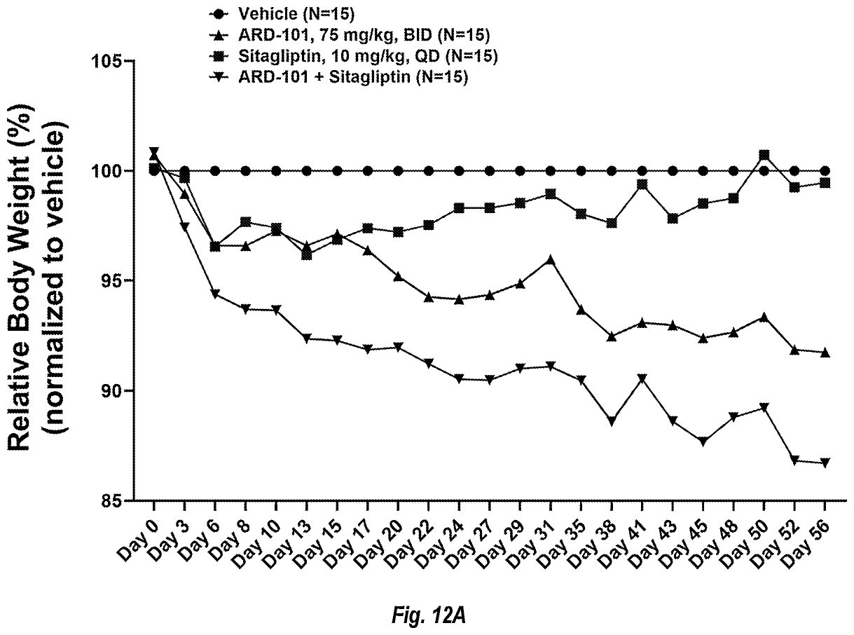

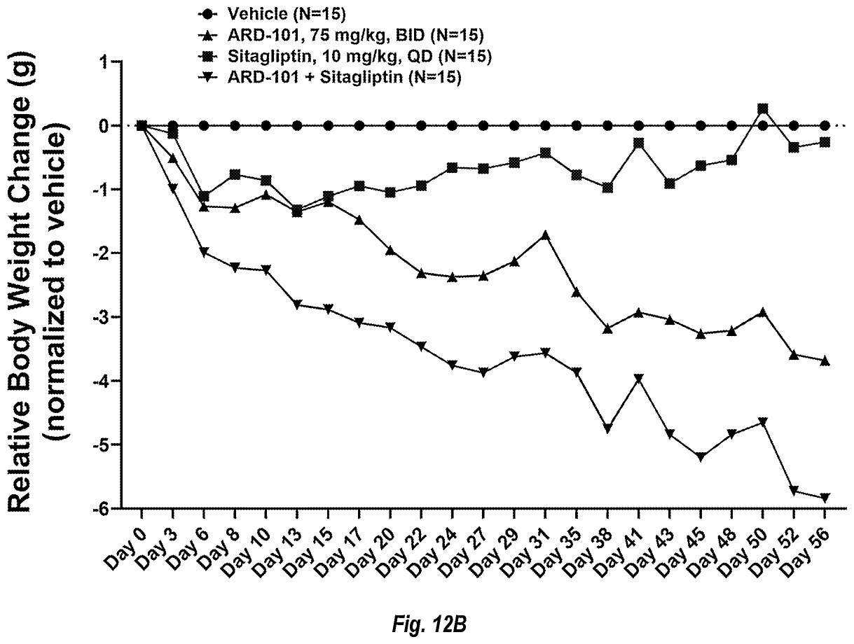

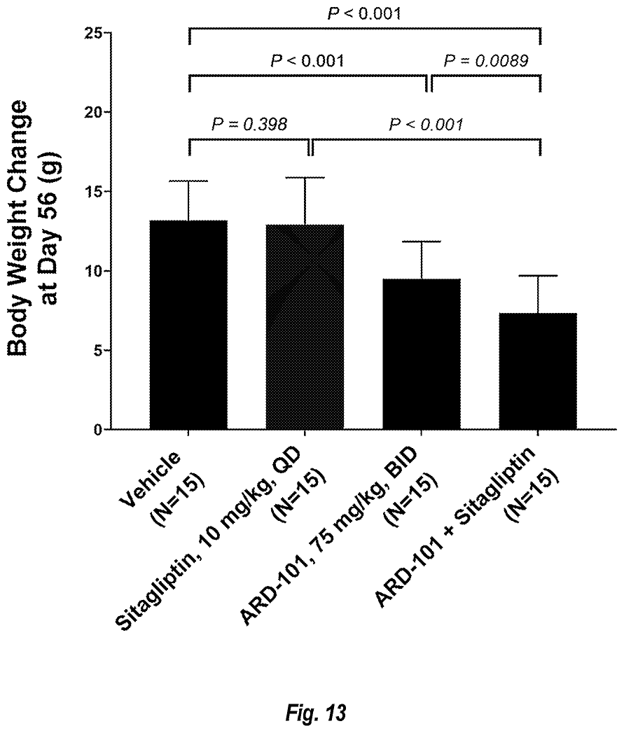

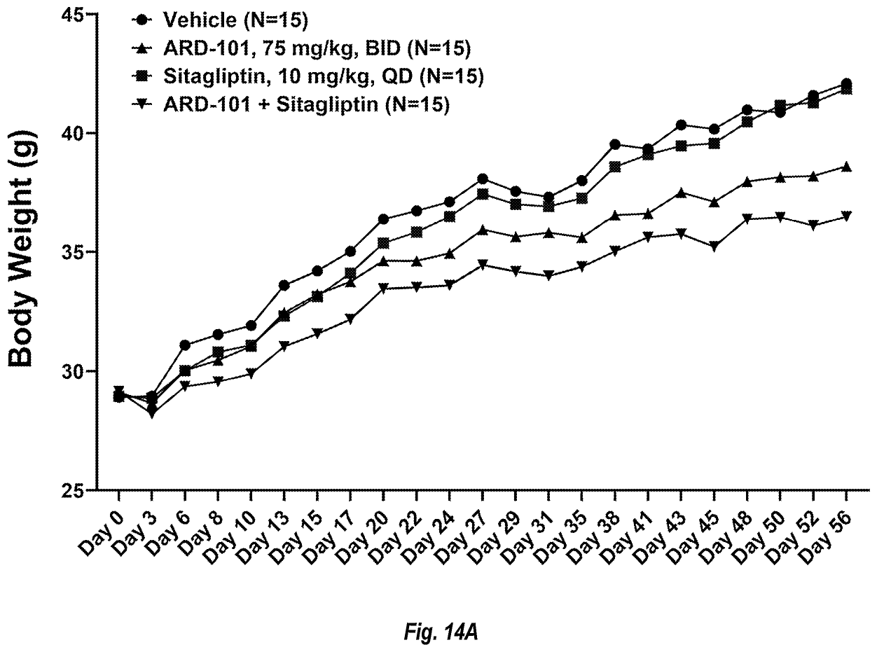

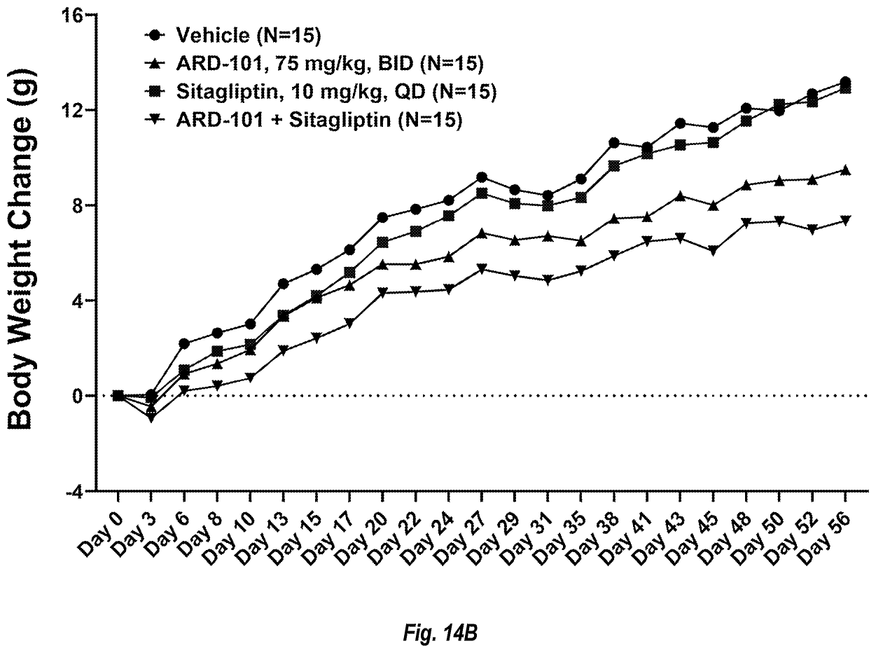

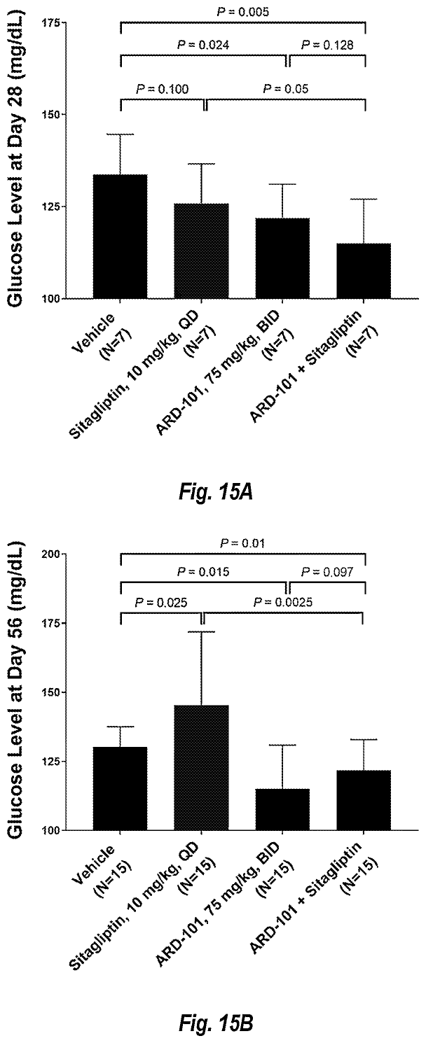

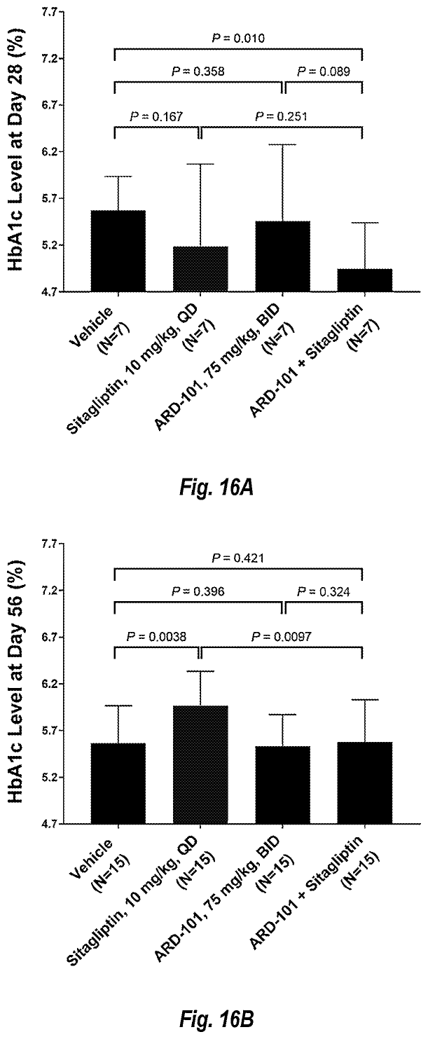

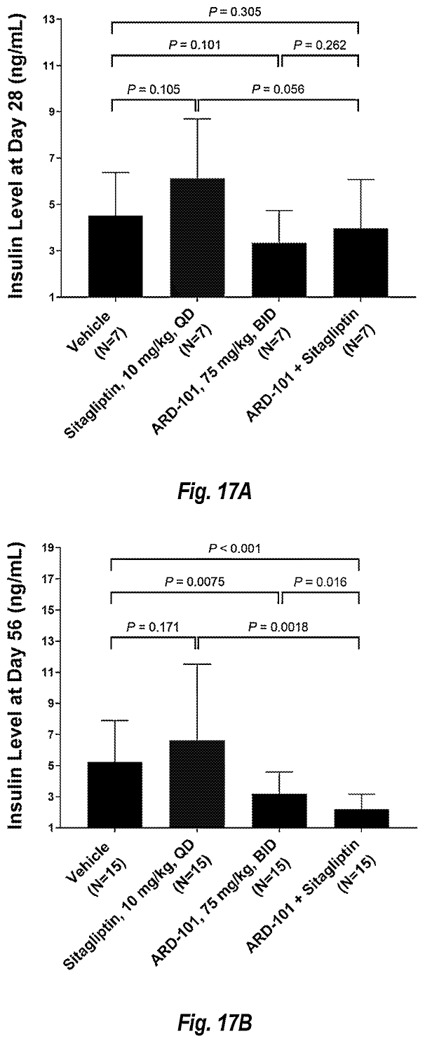

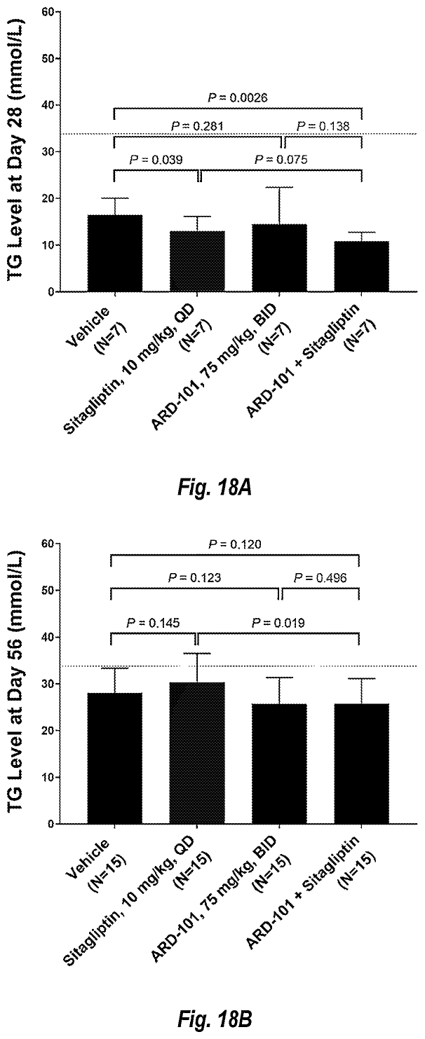

FIGS. 13 through 22B show data from this Example 4, which is further described above under the heading, Brief Description of the Figures. The data from this study showed that the combination of a bitter receptor agonist (denatonium acetate or ARD-101) and a DPP-4 inhibitor (sitagliptin) showed highly synergistic and significant effect for body weight in DIO mice. Given that the DPP-4 inhibitors have not achieved weight loss indications as marketed drugs (but have shown to be effective treatment agents for type 2 diabetes and lowering HbA1c), combining a DPP-4 inhibitor with a bitter receptor agonist, such as denatonium acetate, provides synergistic benefit for weight loss (treating obesity), diabetes/glycemic control and hyperlipidemia.

### Example 5

### Combination Pharmaceutical Composition for Oral Administration

Combining two or more oral antidiabetic agents into a single tablet provides a potential means of delivering combination therapy without adding to the complexity of patients' daily regimens. Such formulations have been well accepted in other disease indications, such as hypertension (HYZAAR™ which is a combination of losartan potassium and hydrochlorothiazide) and cholesterol lowering (VYTORIN™ which is a combination of simvastatin and ezetimibe). Examples of marketed combination tablets containing two oral antidiabetic agents include metformin and a DPP-4 inhibitor sitaglipin (Janumet®), saxagliptin (Kombiglyze®), linagliptin (Jentadueto®), and alogliptin (Kazano®).

This Example provides a formulation of DA Tablet Admixed With a DPP-4 Inhibitor providing that the DPP-4 inhibitor is released just before the denatonium salt so that circulating DPP-4 inhibitor is able to increase the half-life of GLP-1 and PYY gut peptide hormones stimulated for release by the denatonium salt in the small intestine.

This Example provides an immediate release 100 mg particle formulation of denatonium acetate (DA) as a free base as an immediate gastric release oral pharmaceutical formulation and a non-granule water-soluble DPP-4 inhibitor. In one embodiment, the immediate release comprises release in the stomach or gut to avoid or minimize oral cavity exposure. This embodiment provides the advantage of avoiding subjective taste aversion to the API.

The detailed manufacturing steps are described below.

### 1.-3. Drug Layer/Seal Coating/Final Blending.

The drug layering, seal coating, and final blending processes as described above in Example 1B were performed to produce blended seal coated beads, Denatonium IR Pellets, used for encapsulation.

### 4. Encapsulation—Denatonium Capsules, 100 mg

The Denatonium IR pellets (100 mg), were filled into size 1, white opaque hard gelatin capsules using an auto capsule filling machine. Capsules were then passed through an in-line capsule polisher and metal detector. In-process controls for capsule weight and appearance was performed during the encapsulation process. Acceptable quality limit (AQL) sampling and testing was performed by Quality Assurance (QA) on a composite sample during the encapsulation process. Finished product composite sample was collected and analyzed as per specification for release testing.

5. Packaging—Capsules, 100 mg—30 Counts

The 100 mg capsules were packaged in 30 counts into 50/60 cc White HDPE round S-line bottles with 33 mm White CRC Caps. The bottles were torqued and sealed using an induction sealer.

Table 9 shows qualitative and quantitative formulation composition of the DA/DDP4i combination capsule according to this Example.

TABLE 9

| | | | | | Limits based on IID | |
|---|---|---|---|---|---|---|
| | | | | | Max Potency | |
| | Quality | | Quantity (%) | DA capsule- | for Unit | |
| Ingredient | Standard | Function | w/w | 100 mg (mg/cap) | Dose (mg) | Reference |
| Denatonium | In-house | API | 23.55 | 118.06 (100 mg | N/A | N/A |
| acetate | | | | Denatonium | | |
| | | | | base) | | |
| DPP-4 | USP | API | 12.50 | 50 mg | | |
| inhibitor** | | | | sitagliptin free | | |
| | | | | acid | | |
| Povidone | USP | Binder | 2.36 | 5.90 | 61.5 | Oral - |
| (KOLLIDON 30) | | | | | | Capsule |
| Sugar Spheres | NF | Substrate | 68.85 | 172.57 | 314.13 | Oral - |
| (VIVAPHARM ® | | | | | | Capsule |
| Sugar Spheres | | | | | | |
| 35-45) | | | | | | |
| Hypromellose | USP | Binder | 3.64 | 9.14 | 150 | Oral - |
| (Methocel E5 | | | | | | Capsule |
| Premium LV | | | | | | |
| Hydroxypropyl | | | | | | |
| Methylcellulose) | | | | | | |
| Talc (Micro Talc | USP | Anti-tacking | 1.09 | 2.74 | 14 | Oral - |
| MP 1538 USP | | agent | | | | Capsule, |
| Talc) | | | | | | coated |
| | | | | | | pellets |
| Talc (extra | USP | Flow aid | 0.50 | 1.25 | 284.38 | Oral - |
| granular) | | | | | | Capsule |
| (Micro Talc MP | | | | | | |
| 1538 USP Talc) | | | | | | |
| Total weight of beads | | | 250.62 | | N/A | N/A |
| Hard Gelatin | USP | Capsule shell | N/A | 73.3 | 107 | Oral - |
| Capsule Shells; | | | | | | Capsule |
| Cap: White | | | | | | |
| Opaque: Body: | | | | | | |
| White Opaque; | | | | | | |
| Size: 1 | | | | | | |

| | | | |
|---|---|---|---|
| Total weight of Filled Capsule | 323.9 | N/A | N/A |

IID, the Inactive Ingredient Database;
API, active pharmaceutical ingredient;
USP, the US Pharmacopeia;
NF, the National Formulary
* Solvents such as Ethyl Alcohol USP 190 Proof (190 Proof Pure Ethyl Alcohol) and purified water (USP) were used for the preparation of drug solution and seal coating dispersion but are removed during the manufacturing process.
**DPP-4 inhibitor selected from the group consisting of a salt of a medium chain fatty acid, a salt of N-(8-(2-hydroxybenzoyl)amino)caprylic acid, sodium N-(8-(2-hydroxybenzoyl)amino)caprylate (SNAC), sitagliptin, saxagliptin, linagliptin, and alogliptin.

A bilayer tablet will contain a first layer of a bitter receptor agonist, preferably a denatonium salt and a second layer of a DPP-4 inhibitor. The DPP-4 inhibitor is selected from the group consisting of sitagliptin, vildagliptin, saxagliptin, P93/01, SYR322, GSK 823093, Roche 0730699, TS021, E3024, and PHX-1149. Preferably, the DPP-4 inhibitor is alogliptin, carmegliptin, melogliptin, dutogliptin, denagliptin, linagliptin, sitagliptin, vildagliptin, or saxagliptin. In a subclass of this class, the DPP-4 inhibitor is sitagliptin.

A preferred pharmaceutically acceptable salt of sitagliptin is a dihydrogen phosphate salt (sitagliptin phosphate). A preferred form of the sitagliptin dihydrogen phosphate salt is a crystalline monohydrate (sitagliptin phosphate monohydrate) disclosed in WO 2005/0031335, the disclosure of which is incorporate by reference herein.

The preparation of sitagliptin phosphate monohydrate is disclosed in international patent publication WO 2005/0031335 published on Jan. 13, 2005, the contents of which are incorporated by reference.

The dosage strength of the DPP-4 inhibitor for incorporation into the pharmaceutical compositions is an amount from about 1 milligram to about 250 milligrams of the active moiety. A preferred dosage strength of the DPP-4 inhibitor is an amount from about 25 milligrams to about 200 milligrams of the active moiety. Discrete dosage strengths are the equivalent of 25, 50, 75, 100, 150, and 200 milligrams of the DPP-4 inhibitor active moiety. By “active moiety” is meant the free base form of the DPP-4 inhibitor as an anhydrate.

The unit dosage strength of sitagliptin free base anhydrate (active moiety) for inclusion into the fixed-dose combination pharmaceutical compositions is 25, 50, 75, 100, 150, or 200 milligrams. A preferred dosage strength of sitagliptin is 50 (for BID) or 100 milligrams daily. An equivalent amount of sitagliptin phosphate monohydrate to the sitagliptin free base anhydrate is used in the pharmaceutical compositions, namely, 32.13, 64.25, 96.38, 128.5, 192.75, and 257 milligrams, respectively.

The dosage strength of the denatonium salt is administered one per day or twice per day at a total daily dose (per weight of denatonium) of from about 50 mg to about 3000 mg, preferably from about 100 mg to about 2000 mg, and most preferably from about 200 mg to about 1000 mg.

The pharmaceutical composition comprises:
(a) a second layer comprising about 20 to 45% by weight of a dipeptidyl peptidase-4 inhibitor, or a pharmaceutically acceptable salt thereof; and
(b) a first layer comprising about 7 to 24% by weight of a bitter receptor agonist.

The second layer additionally comprises one or more excipients selected from the group consisting of: (i) a diluent; (ii) a disintegrant; and (iii) a lubricant. In a subclass of this class, the first layer additionally comprises one or more excipients selected from the group consisting of (i) two diluents; (ii) a disintegrant; and (iii) two lubricants.

The second layer additionally comprises one or more excipients selected from the group consisting of: (i) about 40-80% by weight of a diluent; (ii) about 0.5-6% by weight of a disintegrant; and (iii) about 0.75-10% by weight of a lubricant. In a subclass of this class, the second layer additionally comprises one or more excipients selected from the group consisting of: (i) about 40-80% by weight of two diluents; (ii) about 0.5-6% by weight of a disintegrant; and (iii) about 0.75-10% by weight of two lubricants.

Alternatively, the second layer additionally comprises one or more excipients selected from the group consisting of: (i) about 20-40% by weight of a first diluent; (ii) about 20-40% of a second diluent; (iii) about 0.5-6% by weight of a disintegrant; (iv) about 0.25-4% by weight of a first lubricant and (v) about 0.5-6% by weight of a second lubricant. In a subclass of this class, the first diluent is microcrystalline cellulose; the second diluent is anhydrous dibasic calcium phosphate; the disintegrant is croscarmellose sodium; the first lubricant is magnesium stearate; and the second lubricant is sodium stearyl fumarate.

The dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

Alternatively, the pharmaceutical composition comprises:
(a) a second layer comprising: (i) about 20 to 45% by weight of a dipeptidyl peptidase-4 inhibitor, or a pharmaceutically acceptable salt thereof; (ii) about 40-80% by weight of a diluent; (iii) about 0.5-6% by weight of a disintegrant; and (iv) about 0.75-10% by weight of a lubricant; and
(b) a first layer comprising: (i) about 7 to 24% by weight of a denatonium salt; (ii) about 60-80% by weight of a diluent; (iii) about 2-12% by weight of a disintegrant; (iv) about 1-7% by weight of a binding agent, and (v) about 0.25-4% by weight of a lubricant.

The dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

### Combination Therapies for Glycemic Control Diabetes

Table 10 shows a comparison of denatonium acetate (DA) with DPP-4 inhibitors in similar ob/ob mice model. The data for the DPP-4 inhibitors was obtained from J. Clin. Biochem. Nutr. 2015; 57(3):244-53. Acta Pharmacol. Sin. 2012; 33(8): 1013-22. J. Pharmacol. Exp. Ther. 2012; 342(1): 71-80; and Eur. J. Pharmacol. 2008; 588(2-3):325-32.

TABLE 10

| | | | Vildagliptin | | |
|---|---|---|---|---|---|
| | | | (Galvus ®)(not | | |
| | | Sitagliptin | launched in | Linagliptin | Alogliptin |
| Product Name | DA | (Januvia ®) | U.S.) | (Tradjenta ®) | (Nesina ®) |
| Animal Model | ob/ob mice fed | ob/ob mice fed | ob/ob mice | ob/ob mice | ob/ob mice |
| (No. per group) | with high-fat | with high- | (N = 11) | (N = 10) | (N = 7) |
| | diet (N = 14) | fructose diet | | | |
| | | (N = 6) | | | |
| Dose and | 50 mg/kg, BID | 0.0018% | 1.9, 6.3, and 19 | 3 mg/kg, QD for | 42.2 mg/kg/day |
| Schedule | for 8 weeks | sitagliptin in diet | mg/kg, BID for | 12 days | for 28 days |
| (HED)* | (240 mg, BID) | for 12 weeks | 33 days | (14.6 mg, QD) | (205 mg, QD) |
| | | (~14 mg, QD) | (9.2, 30.6, and | | |
| | | | 92 mg, BID) | | |
| Clinical Highest | 240 mg, BID | 100 mg, QD | 50 mg, BID | 5 mg, QD | 25 mg, QD |
| Dose | | | | | |
| Body Weight | No significant | No significant | No significant | No significant | No significant |
| Gain | effect | effect | effect | effect | effect |
| Control-adjusted | −29.3%* | −23.4%* | −29.4%, −40.2%**, | N/A | −15.6% |
| Percent Change | | | and −43.1%** | | |
| in Fasting Blood | | | | | |
| Glucose | | | | | |
| Control-adjusted | +0.8% | N/A | N/A | N/A | −13%** |
| Percent Change | | | | | |
| in HbAlc Level | | | | | |
| Control-adjusted | +4.3% | +148%* | N/A | N/A | +105%* * |
| Percent Change | | | | | |
| in Insulin Level | | | | | |
| Control-adjusted | −21.2%* | −43.2%* | −29.3%**, −19.9%**, | N/A | −24.5% |
| Percent Change | | | and −26.2%** | | |
| in TG Level | | | | | |
| Control-adjusted | −27.5%* | N/A | No significant | N/A | N/A |
| Percent Change | | | effect on total | | |
| in LDL Level | | | cholesterol | | |

*, ** , and *** represent p < 0.05, <0.01, and <0.001 vs. control, respectively.

Similarly, Table 11 provides a comparison of denatonium acetate (DA) with DPP-4 inhibitors in similar ob/ob mice model. The data for the DPP-4 inhibitors was obtained from Am. J. Physiol. Endocrinol. Metab. 2011; 300(2); E410-E421. Biochim. Biophys. Acta Gen. Subj. 2018; 1862(3):403-413. PLOS One. 2012; 7(6):e38744; and Aging Cell. 2019; 18(2):e12883.

TABLE 11

| | | | Vildagliptin | | |
|---|---|---|---|---|---|
| | | | (Galvus ®) | | |
| | | Sitagliptin | (not launched | Linagliptin | Alogliptin |
| Product Name | DA | (Januvia ®) | in U.S.) | (Tradjenta ®) | (Nesina ®) |
| Animal Model | C57B1/6J | C57B1/6J mice | C57B1/6J mice | C57B1/6J mice | C57B1/6J mice |
| (No. per | mice fed with | fed with high- | fed with high- | fed with high- | fed with high- |
| group) | high-fat diet | fat diet | fat diet | fat diet | fat diet |
| | (N = 15) | (N = 10) | (N = 14) | (N = 15) | (N = 55) |
| Dose and | 75 mg/kg, | 0.004% | 1. 30 mg/kg/ | 3 mg/kg and | 0.03% (wt/wt) |
| Schedule | BID for 8 | sitagliptin in | day for 7 weeks | 30 mg/kg for | in diet for life |
| (HED)* | weeks | diet for 12 | 2. (~146 mg, QD) | 4 weeks | span |
| | (360 mg, BID) | weeks | | (14.6 mg and | (233 mg, QD) |
| | | (~30 mg, QD) | | 146 mg, QD) | |
| Clinical | 240 mg, BID | 100 mg, QD | 50 mg, BID | 5 mg, QD | 25 mg, QD |
| Highest Dose | | | | | |
| Control- | −33.9%* | −45.8%* | +11% | −0.2% and | −32.6%* |
| adjusted | | | | +0.7% | |
| Percent | | | | | |
| Change in | | | | | |
| Body Weight | | | | | |
| Gain | | | | | |
| Control- | −5.3% | −20.9%* | −15.1%* | −6.5% *** and | −10.1% |
| adjusted | | | | −8% *** | |
| Percent | | | | | |
| Change in | | | | | |
| Fasting Blood | | | | | |
| Glucose | | | | | |
| Control- | −3.7%* (at the | N/A | N/A | −6.5%*** | −8.8% |
| adjusted | end of Week 4) | | | and −6.5%*** | |
| Percent | | | | | |
| Change in | | | | | |
| HbAlc Level | | | | | |
| Control- | −42.9% | −41.7% | −33.2% | N/A | -47.9%* |
| adjusted | | | | | |
| Percent | | | | | |
| Change in | | | | | |
| Insulin Level | | | | | |
| Control- | −18.3% in LDL | N/A | −23.6% in TG** | N/A | −19.2% in TG* |
| adjusted | | | −51.5% in TC* | | −1.1% in TC |
| Percent | | | | | −16.4% in LDL |
| Change in | | | | | |
| Lipid Levels | | | | | |
| Control- | +27.5%* | N/A | N/A | 18.4 pM and | N/A |
| adjusted | | | | 26.4 pM vs. | |
| Percent | | | | Not | |
| Change in | | | | Detectable | |
| GLP-1 Level | | | | for control | |
| | | | | group | |

*, **, and ***represent p < 0.05, <0.01, and <0.001 vs. control, respectively.

### Example 5A

This Example provides an oral formulation combination bilayer tablet comprising a fixed dosage of a DPP4 inhibitor and a bitter receptor agonist. Preferably the bilayer ingredients are sitagliptin and denatonium acetate (200 mg per bilayer tablet) and sitagliptin (50 mg per bilayer tablet) designed for either one bilayer tablet or two monolayer tablets to be administered (at least 6 hours apart) per day or BID. The in vivo data provided in Example 4 herein shows the synergistic effect of this combination wherein a combination tablet can be administered, or separate tablets administered together.

Sitagliptin dihydrogen phosphate monohydrate is an orally-active inhibitor of the DPP-4 enzyme, chemically designated as 7-[(3R)˜ 3-amino-1-oxo-4-(2,4,5-trifluorophenyl)butyl]-5,6,7,8-tetrahydro-3-(trifluoromethyl)-1,2,4-triazolo[4,3-a]pyrazine phosphate (1:1) monohydrate. It is indicated as an adjunct therapy to diet and exercise to improve glycemic control in adults with type 2 diabetes mellitus. However, sitagliptin does not achieve weight loss.

U.S. Pat. No. 7,326,708 (incorporated by reference herein), in particular Example 7, discloses a process for the preparation of a sitagliptin phosphate salt. The film-coated tablets Januvia® are being marketed by Merck in the USA. The Januvia® tablet contains 32.13, 64.25, or 128.5 mg of sitagliptin phosphate monohydrate, which is equivalent to 25, 50, or 100 mg, respectively, of free base.

A preferred combination oral dosage form contains two drug compartments, layered on top of each other. The denatonium acetate compartment has a compressed particle formulation

This provides an immediate release 200 mg particle formulation of denatonium acetate (DA) as an immediate gastric release oral pharmaceutical formulation and a non-particle water-soluble DPP-4 inhibitor.

Table 12 shows qualitative and quantitative formulation composition of DA in this Example.

TABLE 12

| | | | | | Limits based on IID | |
|---|---|---|---|---|---|---|
| | | | | | Max Potency | |
| | Quality | | Quantity (%) | DA capsule- | for Unit | |
| Ingredient | Standard | Function | w/w | 100 mg (mg/cap) | Dose (mg) | Reference |
| Denatonium | In-house | API | 47 | 59.03 (100 mg | N/A | N/A |
| acetate | | | | Denatonium | | |
| | | | | base) | | |
| Povidone | USP | Binder | 2.36 | 5.90 | 61.5 | Oral - |
| (KOLLIDON 30) | | | | | | Capsule |
| Sugar Spheres | NF | Substrate | 68.85 | 172.57 | 314.13 | Oral - |
| (VIVAPHARM ® | | | | | | Capsule |
| Sugar Spheres | | | | | | |
| 35-45) | | | | | | |
| Hypromellose | USP | Binder | 3.64 | 9.14 | 150 | Oral - |
| (Methocel E5 | | | | | | Capsule |
| Premium LV | | | | | | |
| Hydroxypropyl | | | | | | |
| Methylcellulose) | | | | | | |
| Talc (Micro Talc | USP | Anti-tacking | 1.09 | 2.74 | 14 | Oral - |
| MP 1538 USP | | agent | | | | Capsule, |
| Talc) | | | | | | coated |
| | | | | | | pellets |
| Talc (extra | USP | Flow aid | 0.50 | 1.25 | 284.38 | Oral - |
| granular) | | | | | | Capsule |
| (Micro Talc MP | | | | | | |
| 1538 USP Talc) | | | | | | |
| Total weight of beads | | | 250.62 | | N/A | N/A |
| Hard Gelatin | USP | Capsule shell | N/A | 73.3 | 107 | Oral - |
| Capsule Shells; | | | | | | Capsule |
| Cap: White | | | | | | |
| Opaque: Body: | | | | | | |
| White Opaque; | | | | | | |
| Size: 1 | | | | | | |

| | | | |
|---|---|---|---|
| Total weight of Filled Capsule | 323.9 | N/A | N/A |

IID, the Inactive Ingredient Database;
API, active pharmaceutical ingredient;
USP, the US Pharmacopeia;
NF, the National Formulary
* Solvents such as Ethyl Alcohol USP 190 Proof (190 Proof Pure Ethyl Alcohol) and purified water (USP) were used for the preparation of drug solution and seal coating dispersion but are removed during the manufacturing process.

The detailed manufacturing steps are described below.

### 1.-3. Drug Layer/Seal Coating/Final Blending.

The drug layering, seal coating, and final blending processes as described above in Example 1B were performed to produce blended seal coated beads, Denatonium IR Pellets, used for compression into tablets.

### 4. Tablet Compression—Denatonium/DPP4i Tablets, 100 mg

The Denatonium IR pellets, 100 mg, were compressed into a tablet layer to be layered on top of a DPP-4 inhibitor tablet layer described below.

A second layer comprises a dipeptidyl peptidase-4 inhibitor, or a pharmaceutically acceptable salt thereof. The second bilayer additionally comprises one or more excipients selected from the group consisting of: (i) a diluent; (ii) a disintegrant; and (iii) a lubricant. In another embodiment of the present invention. The second bilayer additionally comprises one or more surfactants or wetting agents; and one or more antioxidants.

The pharmaceutical bilayer compositions are prepared by dry and wet processing methods. A DA layer is prepared by wet processing methods, preferably wet granulation methods. With wet granulation either high-shear granulation or fluid-bed granulation may be used. Alternatively, the DA layer is prepared by fluid-bed granulation. Fluid bed granulation processing has the advantage of affording tablets with higher diametric strength. The wet processing methods enhance the chemical stability of DA. Alternatively, the DPP-4 layer is prepared by dry processing methods. In a class of this embodiment, the DPP-4 inhibitor layer is prepared by direct compression. Additionally, using a bilayer tablet with a separate DA layer containing a disintegrant, such as crospovidone, further increases stability of the tablet.

The pharmaceutical compositions obtained by dry and wet processing methods may be compressed into tablets, encapsulated, or metered into sachets.

The pharmaceutical compositions contain one or more lubricants or glidants. Examples of lubricants include magnesium stearate, calcium stearate, stearic acid, sodium stearyl fumarate, hydrogenated castor oil, and mixtures thereof. In one embodiment, the lubricant is magnesium stearate or sodium stearyl fumarate, or a mixture thereof. Or the lubricant is magnesium stearate or sodium stearyl fumarate. Examples of glidants include colloidal silicon dioxide, calcium phosphate tribasic, magnesium silicate, and talc.

The pharmaceutical bilayer tablet compositions optionally contain one or more binding agents. Embodiments of binding agents include hydroxypropylcellulose (HPC), hydroxypropylmethyl cellulose (HPMC), hydroxyethyl cellulose, starch 1500, polyvinylpyrrolidone (povidone), co-povidone, and polyvinylpyrrolidone.

The pharmaceutical bilayer tablet compositions may also optionally contain one or more diluents. Examples of diluents include mannitol, sorbitol, anhydrous dibasic calcium phosphate, lactose monohydrate, dibasic calcium phosphate dihydrate, microcrystalline cellulose, powdered cellulose, and combinations thereof. An example of a combination is mannitol, anhydrous dibasic calcium phosphate, lactose monohydrate and microcrystalline cellulose, or a mixture of any two, three or four thereof. Another example of a diluent combination is selected from: anhydrous dibasic calcium phosphate, lactose monohydrate and microcrystalline cellulose, or a mixture of any two or three thereof. Microcrystalline cellulose is available from several suppliers and includes Avicel, Avicel PH 101, Avicel PH 102, Avicel, PH 103, Avicel PH 105, and Avicel PH 200, manufactured by the FMC Corporation. Another example of a diluent is a mixture of microcrystalline cellulose and mannitol, wherein the diluent is a 2:1 to 1:2 mixture of microcrystalline cellulose to mannitol.

The pharmaceutical bilayer tablet compositions may also optionally contain a disintegrant. The disintegrant may be one of several modified starches, modified cellulose polymers, or polycarboxylic acids, such as croscarmellose sodium, sodium starch glycolate, polacrillin potassium, carboxymethylcellulose calcium (CMC Calcium), and crospovidone.

The pharmaceutical bilayer tablet compositions may also optionally contain one or more surfactants or wetting agents. The surfactant may be anionic, cationic, or neutral. Anionic surfactants include sodium lauryl sulfate, sodium dodecanesulfonate, sodium oleyl sulfate, and sodium laurate mixed with stearates and talc. Cationic surfactants include benzalkonium chlorides and alkyltrimethylammonium bromides. Neutral surfactants include glyceryl monooleate, polyoxyethylene sorbitan fatty acid esters, polyvinyl alcohol, and sorbitan esters. Embodiments of wetting agents include poloxamer, polyoxyethylene alkyl ethers, polyoxyethylene castor oil derivatives, and polyoxyethylene stearates.

The pharmaceutical bilayer tablet compositions may also optionally contain an anti-oxidant which may be added to the formulation to impart chemical stability. The anti-oxidant is selected from the group consisting of α-tocopherol, γ-tocopherol, δ-tocopherol, extracts of natural origin rich in tocopherol, L-ascorbic acid and its sodium or calcium salts, ascorbyl palmitate, propyl gallate, octyl gallate, dodecyl gallate, butylated hydroxytoluene (BHT), and butylated hydroxyanisole (BHA). In one embodiment, the antioxidant is BHT or BHA.

Preferred dosage forms for the pharmaceutical compositions are tablets which are prepared by compression methods. Such tablets may be film-coated such as with a mixture of hydroxypropylcellulose and hydroxypropylmethylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; or any other suitable immediate-release film-coating agent(s). The coat provides taste masking and additional stability to the final tablet. A commercial film-coating agent is Opadry® which is a formulated powder blend provided by Colorcon. Embodiments of Opadry® useful in the present invention include, but are not limited to, Opadry® I (HPC/HPMC), Opadry® 20A18334, Opadry® II, Opadry® II HP (PVA-PEG), or another suitable Opacity® suspension (such as polyvinyl alcohol, polyethylene glycol, titanium dioxide, and talc, with or without colorants).

Finally, a sweetening agent and/or flavoring agent may be added if desired.

### Example 6

This Example provides the results obtained from an in vivo mouse model of fatty liver disease treatment to investigate the therapeutic effect of DA on the treatment of NASH versus positive control semaglutide (a GLP-1 agonist marketed drug for lowering HbA1c). This study used a positive control semaglutide, a vehicle control, study drug DA and a combination of semaglutide and DA. Mouse strain (B6 mice) was used, and the animals were already adults (23 weeks old) when the study began because the animals were already fed an AMLN diet for 17 weeks prior to the study being initiated. The study dose began at 75 mg/kg BID. However, after two weeks of dosing, it was found that this DA dose was not well tolerated, so it was lowered to 50 mg/kg BID for the remaining 10 weeks of dosing (a total of 12 weeks).

The study included 3 groups of 10 mice each, (A) vehicle control with distilled water by gavage BID, (B) DA by gavage BID, and (C) semaglutide 10 mmol/kg sc QD. Body weights and changes were measured 3× per week. Serum metabolic markers (blood glucose, blood insulin, HbA1c, HDL, LDL triglycerides and bile acids) were measured at beginning of dosing (baseline) and end of study. At the end of the study, histopathology of liver samples and serum levels of inflammatory biomarkers (IL-6, TNFα, CK-18 and TGF-β) were evaluated. Histopathology was performed blindly with a scoring scale according to NAFLD Activity Score and Fibrosis Score according to Table 13. Table 14 identifies the kits and equipment used to measure serum parameters.

TABLE 13
Nonalcoholic fatty liver disease activity score and
fibrosis score for histopathological assessment

| NAFLD Activity Score | | Lobular | Ballooning |
|---|---|---|---|
| Score | Steatosis | inflammation | degeneration |
| 0 | <5% | None | None |
| 1 | 5-33% | <2 foci/20x field | Few |
| 2 | >33-66% | 2-4 foci/20x field | Many |
| 3 | >60% | >4 foci/20x field | |

| Stage | Histological findings |
|---|---|
| 1a | Mild pericellular fibrosis (only seen on connective tissue stain) |
| 1b | Moderate pericellular fibrosis (readily seen on H&E) |
| 1c | Portal/periportal fibrosis without pericellular fibrosis |
| 2 | Pericellular and portal/periportal fibrosis |
| 3 | Bridging fibrosis |
| 4 | Cirrhosis |

TABLE 14
serum parameter kits and equipment

| Parameter | Matrix | Kit/Equipment Name | Vendor | Catalog No. |
|---|---|---|---|---|
| ALT | Serum | Alanine Aminotransferase Activity | Sigma-Aldrich | MAK052 |
| | | Assay Kit (Sigma-Aldrich) | | |
| AST | Serum | Aspartate Aminotransferase Activity | Sigma-Aldrich | MAK055 |
| | | Assay Kit | | |
| ALB | Serum | Albumin (BCG) Assay Kit | abcam | ab235628 |
| | | (Colorimetric) | | |
| BA | Serum | Mouse Total Bile Acids Kit | Crystal Chem | 80470 |
| Cytokines | Plasma | MILLIPLEX MAP Mouse | Millipore-Sigma | MCYTMAG70PMX32BK |
| | | Cytokine/Chemokine Magnetic Bead | | |
| | | Panel - Premixed 32 Plex - | | |
| | | Immunology Multiplex Assay | | |
| FFA | Liver | Free fatty acid quantitation kit | Sigma-Aldrich | MAK044 |
| GLP-1 | Serum | Mouse GLP-1 ELISA Kit | Crystal Chem | 81508 |
| GLP-2 | Serum | Mouse GLP-2 ELISA Kit | Crystal Chem | 81514 |
| HbA1c | Plasma | Mouse Hemoglobin Alc Kit | Crystal Chem | 80310 |
| HDL | Serum | Mouse HDL-Cholesterol Kit | Crystal Chem | 79990 |
| Insulin | Serum | Ultra-Sensitive Mouse Insulin ELISA | Crystal Chem | 90080 |
| | | Kit | | |
| LDL | Serum | Mouse LDL-Cholesterol Kit | Crystal Chem | 79980 |
| TC | Liver | Total Cholesterol and Cholesteryl Ester | Bio Vision | K603-100 |
| | | Colorimetric/Fluorometric Assay Kit | | |
| TGA | Liver, | Triglyceride Assay Kit | abcam | ab65336 |
| | Serum | | | |

ALB, albumin. ALT, Ala aminotransferase. AST, Asp aminotransferase. BA, bile acids. FFA, free fatty acids. GLP-1/2, glucagon-like peptide-1/2. HDL, high-density lipopeptide. LDL, low-density lipopeptide. No. Number. TC, total cholesterol TGA, triglycerides.

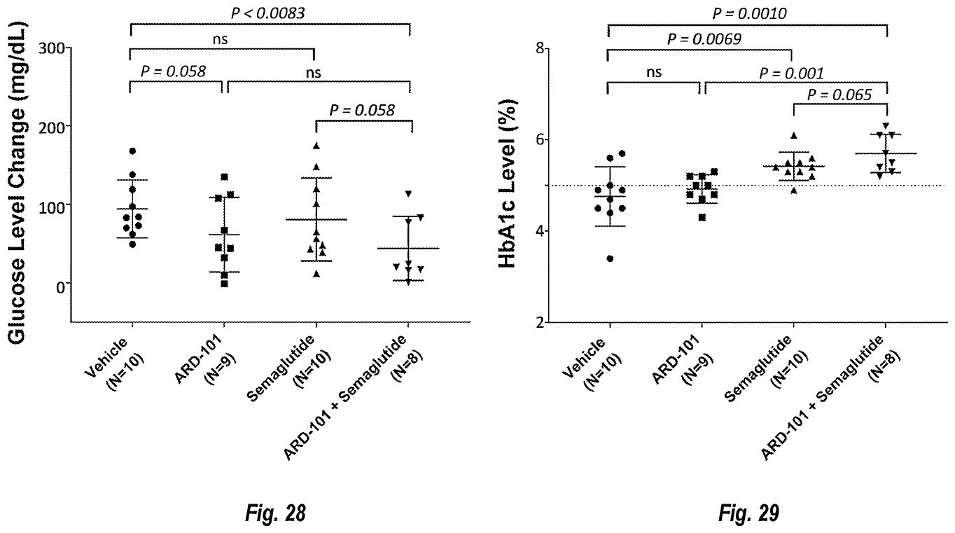

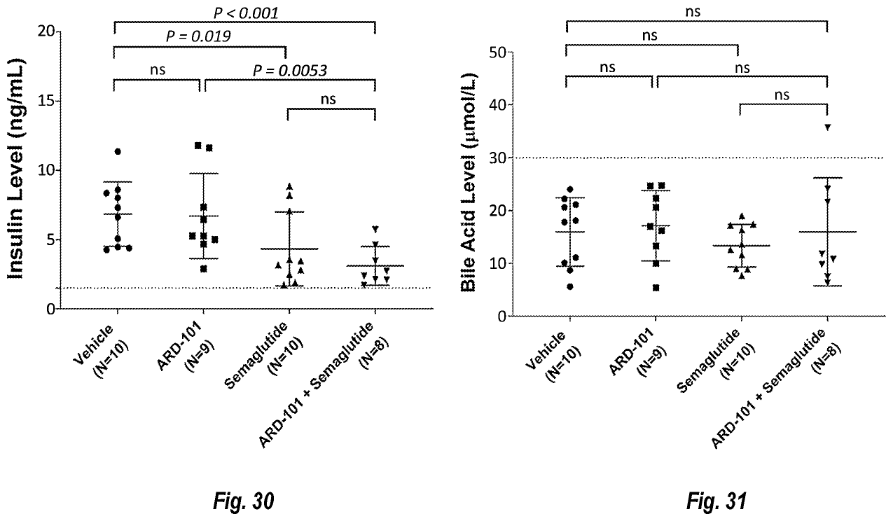

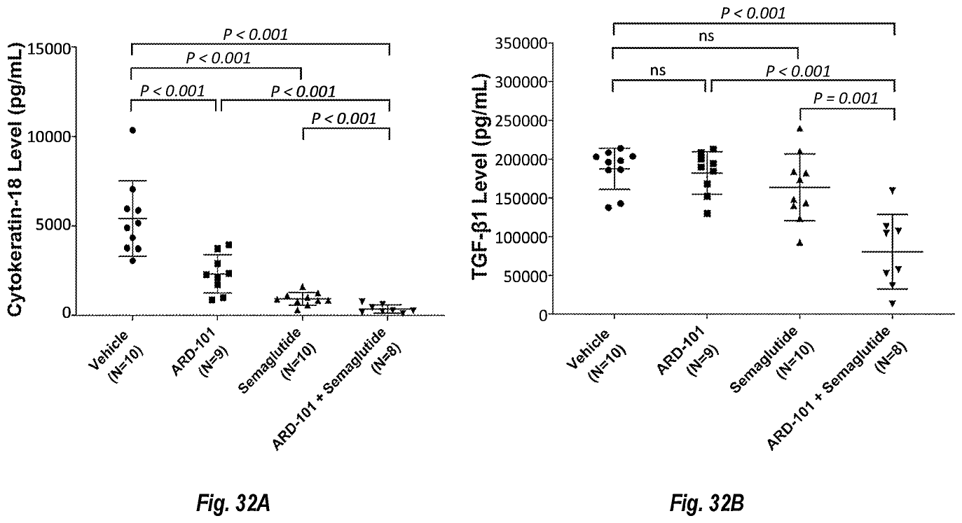

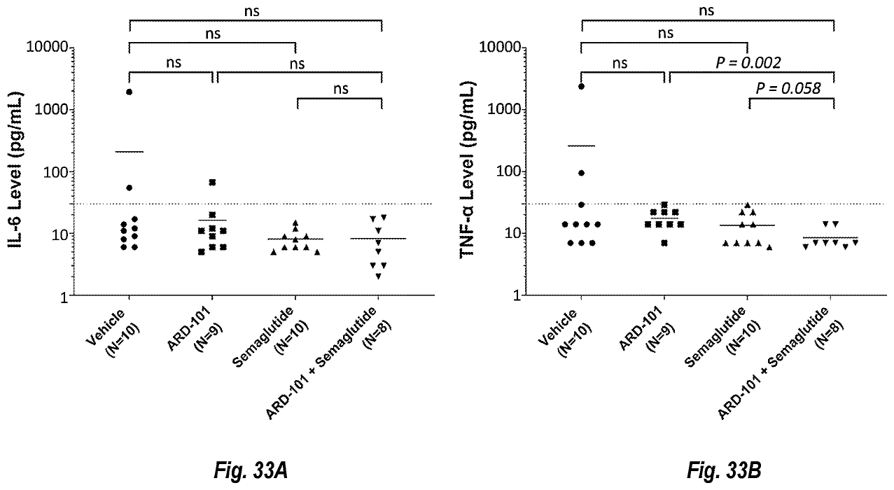

FIGS. 23 through 33B show data from this Example 6, which is further described above under the heading, Brief Description of the Figures. A summary and comparison of NASH in vivo data from multiple studies is shown below in Table 15.

TABLE 15

| | | | | NASH animal | Dose schedule | Findings of | Findings of metabolic |
|---|---|---|---|---|---|---|---|
| | | | | model(s) used in | and treatment | histopathological | biomarker |
| Drug Name | Developer | Modality | MOA | pre-clinical study | period | examination | measurements |
| DA or ARD- | Aardvark | Small | Bitter taste | Male C57BL/6 | 30 mg/kg, PO, QD | Significantly | Lowered serum levels |
| 101 | Thera- | molecule | receptor | mice fed AMLN | from beginning of | decreased liver weight | of fasting glucose |
| | peutics | | agonist | diet (40 kcal % fat, | the study for 48 | of animals (by 10%) | (−9%), HbAlc (−6.7%), |
| | | | | 20 kcal % fructose, | weeks | Significantly improved | ALT (−43%), AST |
| | | | | and 2% | | steatosis and fibrosis | (−9.5%), and bile acid |
| | | | | cholesterol) for 48 | | on histopathology | (−16.2%) |
| | | | | weeks | | | Increased serum levels |
| | | | | | | | of GLP-1 and GLP-2 |
| | | | | | | | Lowered serum levels |
| | | | | | | | of inflammatory |
| | | | | | | | markers |
| MET409 | Metacrine | Small | Farnesoid X | Male C57BL/6 | 10 mg/kg, PO, QD | Significantly improved | Lowered plasma C4, |
| | | molecule | receptor | mice fed AMLN | after 34-week | steatosis with all | liver TG, ALT, and |
| | | | (FXR) | diet for 34 weeks | NASH induction | treatment courses and | AST |
| | | | agonist | | for 2, 4, or 8 | inflammation with 4- | |
| | | | | | weeks | or 8-week treatment | |
| | | | | | | Significant | |
| | | | | | | improvement on | |
| | | | | | | fibrosis only observed | |
| | | | | | | with 8-week treatment | |
| Cenicriviroc | Takeda | Small | CCR2/CCR5 | Male C57BL/6N | 10 mg/kg/day and | Significantly improved | Inhibited intrahepatic |
| | and | molecule | Inhibitor | mice fed choline- | 30 mg/kg/day for | fibrosis on | accumulation of |
| | Tobira | | | deficient, L-amino | 4 weeks and 20 | histopathology only | inflammatory |
| | Thera- | | | acid-defined, high- | mg/kg/day and 30 | with 30 mg/kg/day for | macrophages with 30 |
| | peutics | | | fat diet | mg/kg/day for 14 | 14 weeks | mg/kg/day for 4 or 14 |
| | | | | (CDAHFD)for 4 or | weeks, IP | | weeks, and increased |
| | | | | 14 weeks | | | the frequency of |
| | | | | | | | intrahepatic anti- |
| | | | | | | | inflammatory |
| | | | | | | | macrophages with 30 |
| | | | | | | | mg/kg/day for 14 |
| | | | | | | | weeks |
| Elafibranor | Genfit | Small | PPARα and | Male C57BL/6J | 30 mg/kg, PO, QD | Significantly reduced | No effects on liver TG, |
| (GFT505) | | molecule | PPARδagonist | mice fed with the | for 8 weeks | histopathological | total cholesterol |
| | | | | AMLN diet for 50 | | scores of hepatic | contents, or plasma |
| | | | | weeks | | steatosis and | levels of ALT, AST, |
| | | | | | | inflammation, as well | TG or total cholesterol. |
| | | | | | | as fibrosis severity. | |
| The | Genfit | Small | PPARα and | Male C57BL/6J | Elafibranor (10 | The combination | A strong decrease in |
| combination | | molecule + | PPARδag- | mice fed with the | mg/kg/day, PO) + | decreased the NAS | liver triglycerides |
| of | | Recombinant | onist + | AMLN diet for 35 | Semaglutide (0.3 | score by 3 stages in | (−56%), in the number of |
| elafibranor | | protein | GLP-1 receptor | weeks | nmol/kg, SC) for | 14% of mice, and by 2 | inflammatory foci |
| and | | | agonist | | 12 weeks | stages in 44% of mice. | (−59%) and in plasma |
| semaglutide | | | | | | | ALT (−60%), was also |
| | | | | | | | observed in the |
| | | | | | | | combination group. |
| | | | | | | | Transcriptomic analysis |
| | | | | | | | revealed that both drugs |
| | | | | | | | synergized to |
| | | | | | | | specifically reduce the |
| | | | | | | | inflammatory |
| | | | | | | | infiltration in the liver |
| ALT-801 | Altim- | Recombinant | Dual GLP- | Male C57BL/6J | 5 or 10 nmol/kg, | Significantly | Significantly decreased |
| | mune | protein | 1/glucagon | mice fed with the | SC for 12 weeks | decreased liver weight | liver TG and total |
| | | | receptor | AMLN diet for 32 | | and NAS overall score | cholesterol levels, |
| | | | agonist | weeks | | at both doses | plasma ALT level, and |
| | | | | | | | liver content of fibrosis |
| | | | | | | | markers. |
| Semaglutide | Novo | Recombinant | GLP-1 receptor | Male C57BL/6J | 10 nmol/kg, SC | Significantly | Significantly decreased |
| | Nordisk | protein | agonist | mice fed with the | for 12 weeks | decreased liver weight | liver TG and total |
| | | | | AMLN diet for 32 | | and NAS overall score | cholesterol levels, |
| | | | | weeks | | at 10 nmol/kg | plasma ALT level, and |
| | | | | | | | liver content of fibrosis |
| | | | | | | | markers. |
| Obeticholic | Intercept | Small | FXR agonist | Male C57BL/6J | 30 mg/kg, PO, QD | Significantly reduced | Significantly reduced |
| acid (INT- | Pharma- | molecule | | mice fed with the | for 8 weeks | histopathological | total liver TG and total |
| 747) | ceuticals | | | AMLN diet for 50 | | scores of hepatic | cholesterol, collagen |
| | Inc. | | | weeks | | steatosis and | 1a1, and galectin-3 |
| | | | | | | inflammation. | content. Significantly |
| | | | | | | Significantly reduced | reduced plasma total |
| | | | | | | liver weight. | cholesterol |
| Selonsertib | Gilead | Small | Apoptosis | Male C57BL/6 | Treatment started | Significantly reduced | Reduced serum and |
| (GS-4997) | Sciences | molecule | signal- | mice fed a fast food | after FF diet | liver steatosis, fibrosis, | liver cholesterol by |
| | | | regulating | diet (FF diet) high | induction for 240 | and insulin resistance | 14% and 45%, |
| | | | kinase 1 | in fat, cholesterol, | days. Dose | | respectively, and |
| | | | (ASK1) | and sugar for 330 | unknown | | strongly reduced serum |
| | | | inhibitor | days | | | bile acids including |
| | | | | | | | cholate (91% reduction) |
| | | | | | | | and deoxycholate (90% |
| | | | | | | | reduction). |
| Aramchol | Galmed | Small | Stearoyl-CoA | C57BL/6 male | 5 mg/kg/day PO | Reduced features of | No effects on ALT, |
| | Pharma- | molecule | desaturase-1 | mice fed with the | for the last 2 | steatohepatitis and | AST, or TG. But |
| | ceuticals | | (SCD-1) | methionine- and | weeks | fibrosis | decreased protein |
| | | | inhibitor | choline-deficient | | | expression of COL1A1 |
| | | | | (MCD) diet for 4 | | | in the liver |
| | | | | weeks | | | |
| Pegbelfermin | Bristol- | Recombinant | PEGylated | STAM model: | 3 mg/kg, twice | Significantly | Significantly improved |
| (BMS- | Myers | protein | human | Streptozotocin- | weekly, SC for 2 | decreased mean grades | whole blood glucose |
| 986036) | Squibb | | fibroblast | injected 2-day old | weeks (start at | for steatosis, lobular | (−30%), body weight |
| | | | growth | male C57BL/6 | Week 7) | inflammation, and | (−7.9%), and liver/body |
| | | | factor 21 | mice fed high-fat | | hepatocellular | weight ratio (−20%), as |
| | | | (FGF21) | diet for over 7 | | ballooning | well as liver and plasma |
| | | | analogue | weeks | | | triglycerides. Also |
| | | | | | | | significantly decreased |
| | | | | | | | the mean serum fibrosis |
| | | | | | | | biomarker Pro-C3 by |
| | | | | | | | 44% |
| Emricasan | Conatus | Small | Pan-caspase | Male C57BL/6J | 0.3 mg/kg/day PO | Improved | Decreased serum levels |
| | Pharma- | molecule | inhibitor | mice fed high fat | for 20 weeks | inflammation, | of glucose, HOMA-IR, |
| | ceuticals/ | | | diet for 20 weeks | | ballooning, and | cholesterol, ALT, and |
| | Novartis | | | | | fibrosis on | AST. |
| | | | | | | histopathology | Decreased fibrotic and |
| | | | | | | | inflammatory gene |
| | | | | | | | signature in mice |
| EDP-305 | Enanta | Small | FXR agonist | Male C57BL/6 | 10 mg/kg or 30 | High-dose (30 mg/kg) | High dose significantly |
| | Pharma- | molecule | | mice fed a | mg/kg daily PO | halted fibrosis | decreased fibrogenic |
| | ceuticals | | | CDAHFD diet for | starting from the | progression on | gene expression in |
| | | | | 12 weeks | beginning of week | histopathology in | CDAHFD mice |
| | | | | | 6 | CDAHFD mice | |
| Tropifexor | Novartis | Small | FXR agonist | STAM model: | STAM model: | STAM model: | AMLN model: |
| | | molecule | | Streptozotocin- | 0.03-0.3 mg/kg | tropifexor treatment | tropifexor showed |
| | | | | injected 2-day old | from Week 9 | showed | dose-dependent |
| | | | | C57BL/6J mice fed | AMLN model: | significant decrease in | reduction in ALT/AST |
| | | | | high-fat diet for | 0.03-0.9 mg/kg | NAS at doses ≥0.1 | levels relative to |
| | | | | weeks 4-12 | for the last 4 | mg/kg | controls and |
| | | | | AMLN model: | weeks | AMLN model: | dramatically reduced |
| | | | | C57BL/6J mice | | tropifexor reduced | mRNA levels of |
| | | | | placed on AMLN | | inflammation, | fibrogenic markers and |
| | | | | diet for 30 weeks | | steatosis, and fibrosis | hepatic inflammation |
| | | | | | | in AMLN mice. | cell populations |
| | | | | | | Steatosis and | |
| | | | | | | inflammation were | |
| | | | | | | completely resolved at | |
| | | | | | | doses ≥0.3 mg/kg. | |
| Saroglitazar | Zydus- | Small | PPARα and | Male C57BL/6 | 3 mg/kg PO for 12 | Saroglitazar (3 mg/kg) | Saroglitazar at 3 mg/kg |
| | Cadila | molecule | PPAR γ | mice fed a | weeks (starting | induced reversal of | reduced serum levels of |
| | | | agonist | CDAHFD diet for | from 8 weeks after | hepatic steatosis, | liver damage and |
| | | | | 20 weeks | initiation of the | reduced or no | inflammation markers, |
| | | | | | CDAHFD diet) | vacuolation and | including ALT (60%), |
| | | | | | | ballooning and there | AST (43%), and MCP1 |
| | | | | | | was significant | (45%). Liver lipid (TG) |
| | | | | | | reduction in the | accumulation and |
| | | | | | | severity of | collagen content were |
| | | | | | | inflammation | also significantly (79% |
| | | | | | | | and 41% respectively) |
| | | | | | | | attenuated by |
| | | | | | | | saroglitazar treatment. |
| | | | | | | | Saroglitazar also |
| | | | | | | | reduced liver TNFα |
| | | | | | | | levels and hepatic |
| | | | | | | | fibrogenic and |
| | | | | | | | inflammatory gene |
| | | | | | | | expression in CDAHFD |
| | | | | | | | mice |
| Belapectin | Galectin | Polysac- | Galectin 3 | STAM model: | 60 mg/kg IV twice | Decreased fat | Treatment with GR- |
| (GR-MD-02) | Thera- | charide | inhibitor | Streptozotocin- | a week for 4 | deposition, | MD-02 reduced the |
| | peutics | polymer | | injected 2-day old | weeks starting | hepatocellular | expression of |
| | | | | male C57BL/6 | from Week 6 | ballooning, and | pathological indicators |
| | | | | mice fed high-fat | (early treatment | inflammatory | including iNOS, CD36, |
| | | | | diet for weeks 10- | cohort) or Week 9 | infiltrate, and | and a-smooth muscle |
| | | | | 13 weeks | (late treatment | significantly improved | actin |
| | | | | | cohort). | NAS score in the early | |
| | | | | | | treatment cohort. The | |
| | | | | | | late treatment cohort | |
| | | | | | | GR-MD-02 group | |
| | | | | | | showed improvement | |
| | | | | | | in the NAS versus | |
| | | | | | | vehicle control, | |
| | | | | | | although the value did | |
| | | | | | | not reach significance. | |
| | | | | | | Treatment with GR- | |
| | | | | | | MD-02 markedly | |
| | | | | | | reduced the deposition | |
| | | | | | | of collagen in both the | |
| | | | | | | early and late | |
| | | | | | | treatment cohorts. | |
| Firsocostat | Gilead | Small | Acetyl-CoA | Male C57BL/6 | GS-9674: 10 | The combination | The combination |
| (GS-0976) + | Sciences | molecule | carboxylase | mice fed a fast- | mg/kg, QD, PO | significantly reduced | significantly reduced |
| Cilofexor | | | (ACC) | food diet (FFD) | GS-0976: 0.5 | steatosis on | liver TG and |
| (GS-9674) | | | inhibitor + | enriched in fat, | mg/kg, BID, PO (a | histopathology. | cholesterol levels. |
| | | | Farnesoid X- | cholesterol, and | structural analog | | The combination |
| | | | activated | sugar for 6 months | was used in the | | significantly reduced |
| | | | receptor | | study | | hepatic gene expression |
| | | | agonist | | Treatment started | | of fibrosis and liver |
| | | | | | from Month 5 and | | injury markers, and |
| | | | | | continued for 28 | | plasma level of bile |
| | | | | | days | | acids. |
| Lanifibranor | Inventiva | Small | Pan-PPAR | C57Bl6/J mice fed | 10 or 30 mg/kg, | IVA337 prevented | IVA337 also |
| (IVA337) | Pharma | molecule | (peroxisome | a MCD diet for 3 | PO, QD for 3 | steatosis and | significantly reduced |
| | | | proliferator- | weeks | weeks | inflammation on | plasma alanine |
| | | | activated | | | histopathology. | aminotransferase levels, |
| | | | receptors) | | | | decreased serum as |
| | | | agonist | | | | well as liver |
| | | | | | | | triglyceride levels, and |
| | | | | | | | inhibited the induction |
| | | | | | | | of profibrotic and |
| | | | | | | | fibrotic genes. |
| Resmetirom | Madrigal | Small | Thyroid | C57BL/6 mice | 0.3-3 mg/kg/day | No detectable | Improved insulin |
| (MGL-3196) | Pharma- | molecule | hormone | treated with 60% | PO for 24 days | steatosis, fibrosis or | sensitivity, and reduced |
| | ceuticals | | receptor beta | high fat diet for 36 | | inflammation in | serum levels of ALT |
| | | | agonist | weeks | | MGL-3196 treated | (46%), free fatty acids |
| | | | | | | livers | (30%), and cholesterol |
| | | | | | | | (67%). |
| Azemigli- | Cirius | Small | Mitochondrial | Male | 30 mg/kg/day PO | Treatment with | MSDC-0602K |
| tazone | Thera- | molecule | membrane | DIAMOND ™ | QD for the last 8 | MSDC-0602K | dramatically reduced |
| (MSDC | peutics | | transport | mice fed high fat | weeks (16-24 | significantly decreased | serum levels of fasting |
| 0602K) | | | protein | sugar water diet | weeks) | ballooning; the | insulin, and AST and |
| | | | modulator | (WDSW) for 24 | | steatosis-activity- | ALT. |
| | | | | weeks | | fibrosis (SAF) score | |
| | | | | | | was significantly | |
| | | | | | | lower in the MSDC- | |
| | | | | | | 0602K-treated group | |
| | | | | | | and was reduced to the | |
| | | | | | | level of baseline | |
| | | | | | | controls. The NAFLD | |
| | | | | | | Activity Score (NAS) | |
| | | | | | | trended lower in the | |
| | | | | | | MSDC-0602K-treated | |
| | | | | | | group. The NASH | |
| | | | | | | CRN fibrosis score in | |
| | | | | | | the MSDC-0602K- | |
| | | | | | | treated group was 0. | |
| | | | | | | Significantly fewer | |
| | | | | | | MSDC-0602K-treated | |
| | | | | | | mice progressed from | |
| | | | | | | simple steatosis to | |
| | | | | | | NASH compared to | |
| | | | | | | the control group. | |
| Aldafermin | NGM | Recombinant | FGF19 | FXR-deficient mice | NGM282 | NGM282 reduced | NGM282 reduced |
| (NGM282) | Bio- | protein | analogue | placed on a high- | delivered by | liver weight and | serum levels of ALT |
| | pharma- | | | fat, high-fructose, | adeno-associated | spleen weight in | and AST, and |
| | ceuticals | | | highcholesterol diet | viral vector (AAV) | HFFCD-fed, FXR- | decreased hepatic |
| | | | | (HFFCD) for 50 | at Week 16 | deficient mice | expression of |
| | | | | weeks | | NGM282 did not | inflammatory and |
| | | | | | | impact steatosis in | fibrotic biomarkers |
| | | | | | | HFFCD-fed, FXR- | |
| | | | | | | deficient mice, but | |
| | | | | | | reduced hepatocellular | |
| | | | | | | fibrosis | |
| VK2809 | Viking | Small | Thyroid | A mouse model of | 10 mg/kg/day PO | VK2809 significantly | VK2809 significantly |
| | Thera- | molecule | hormone | diet-induced | for eight weeks | improved NAS score | improved liver contents |
| | peutics | | receptor beta | NASH | | (a 40% mean | of triglyceride, |
| | | | agonist | | | improvement) and | cholesterol, collagen, |
| | | | | | | liver fibrosis on | and hydroxyproline. It |
| | | | | | | histopathology. | also significantly |
| | | | | | | Improvements in all | reduced plasma |
| | | | | | | subcomponents of | triglycerides and |
| | | | | | | NAS (inflammation, | cholesterol. |
| | | | | | | ballooning, and | |
| | | | | | | steatosis) were | |
| | | | | | | observed in VK2809- | |
| | | | | | | treated animals | |

Table 15 shows a wide range of different results in widely different NASH in vivo models. This makes it difficult to do direct comparisons of the data. The study corresponding to the first row (called Aardvark Therapeutics) is provided in PCT Patent application PCT/US2022/014550, filed Jan. 31, 2022.

The studies that emphasized weight loss as a model for NASH treatment seem to be more directed toward treating existing NASH conditions. Therefore, the dosage range of denatonium salt for a method of treatment of NASH and related liver diseases in some embodiments is from about 1.0 mg/kg/day to about 60 mg/kg/day; in some embodiments, from about 2 mg/kg/day to about 40 mg/kg/day; and optionally, from about 4 mg/kg/day to about 20 mg/kg/day. In another embodiment, the dosage range of denatonium salt for a method of prevention and a method of slowing progression of NASH and related liver diseases is from about 0.1 mg/kg/day to about 32 mg/kg/day, preferably from about 0.25 mg/kg/day to about 16 mg/kg/day; and most preferably from about 0.5 mg/kg/day to about 8 mg/kg/day.

### FDA Guidance for Clinical Trials for Treating NASH

On 29 Jan. 2021, the Food and Drug Administration (FDA) gave a short seminar on NASH with fibrosis how treatment drug candidates can show efficacy in animal models and clinical trials. The FDA confirmed that NASH (with fibrosis, hereinafter, NASH) is a serious condition and that a clinical use of surrogate endpoints can predict clinical benefit. Although in animal studies (such as provided in Example 1, herein) histopathological examination is a better proof of treatment, prevention and progression of disease benefit (depending on the length of the animal study). Therefore, in clinical trials, the FDA will accept surrogate endpoints and liver biopsy as means for showing clinical benefit (or lack thereof). The FDA recognized that NASH drug development challenges are due to a gradual and slow progression of chronic inflammatory changes in the liver, and that any NASH drug for prevention of full NASH (advanced liver fibrosis) or treatment or slowing progression are potential lifelong treatments. As for a surrogate endpoint, the FDA has suggested histopathology as “reasonably likely to predict clinical benefit.” The FDA indicated that NASH advanced liver “fibrosis stage, but no other histologic feature of steatohepatitis, has been associated independently with increased mortality, transplantation, and liver-related events.” (citing Angulo et al. Gastroenterology, 149:389-397, 2015).

In conducting clinical trials, the FDA suggests that early-stage trials use noninvasive disease-specific biomarkers (e.g., an aminotransferase), total bilirubin, and radiographic modalities (such as elastography, MRI-PDFF) to assess liver stiffness. For approvals, the FDA will accept improvement in liver histology. “Liver biopsy is a surrogate based on research demonstrating that improvement in histology is likely predictive of an improved clinical outcome in NASH patients.” Liver fibrosis is graded as stage 0 (none), stage, stage 2, stage 3 and stage 4 (cirrhosis). The NASH recommended endpoints are (1) resolution of steatohepatitis AND no worsening of liver fibrosis; OR (2) improvement in liver fibrosis AND no worsening of steatohepatitis; OR (3) both resolution of steatohepatitis and improvement in fibrosis.

We claim:

1. A pharmaceutical composition comprising a combination of a bitter receptor agonist and a gut-signaling compound.

2. The pharmaceutical composition of claim 1, wherein the bitter receptor agonist is a denatonium salt selected from the group consisting of denatonium acetate (DA), denatonium citrate, denatonium maleate, denatonium saccharide, and denatonium tartrate, or is chlorpheniramine, diphenidol, famotidine, haloperidol, quinine, parthenolide, or aristolochic acid.

3. The pharmaceutical composition of claim 1 or 2, wherein the gut-signaling compound is a gut-signaling peptide analog or a gut-signaling hormone enhancer selected from a GLP-1 receptor agonist, a GLP-2 analog, a GLP-IRA analog, a PYY analog, a DPP-4 inhibitor, a GIP analog, and a CCK analog.

4. The pharmaceutical composition of any one of claims 1 to 3, wherein the gut-signaling compound is selected from a salt of a medium chain fatty acid, a salt of N-(8-(2-hydroxybenzoyl)amino)caprylic acid, sodium N-(8-(2-hydroxybenzoyl)amino)caprylate (SNAC), sitagliptin, saxagliptin, linagliptin, alogliptin, semaglutide, glyburide, liraglutide, dulaglutide, albiglutide, NN-9775, JNJ-9321, sitagliptin phosphate, vildagliptin, linagliptin, alogliptin, saxagliptin, P93/01, SYR322, GSK 823093, Roche 0730699, TS021, E3024, PHX-1149, teduglutide, glepaglutide, apraglutide, elsiglutide, HM-15912, ZP-7570, GLP-2-ELP, MOD-1501, or HL-06.

5. The pharmaceutical composition of claims 1 to 4, wherein the bitter receptor agonist is DA.

6. The pharmaceutical composition of any one of claims 1 to 5, wherein the gut-signaling compound is sitagliptin, semaglutide, or liraglutide.

7. The pharmaceutical composition of any one of claims 1 to 6, further comprising a pharmaceutically acceptable carrier.

8. A method for treating a glucagon-related disease, disorder or condition, comprising administering to a subject a combination of a bitter receptor agonist and a gut-signaling compound.

9. Use of a compound comprising a bitter receptor agonist selected from a denatonium salt, chlorpheniramine, diphenidol, famotidine, haloperidol, quinine, parthenolide, and/or aristolochic, the compound being administered as a racemic mixture or as enantiomers, diastereoisomers, or pharmaceutically acceptable salts thereof in combination with a gut-signaling compound, for preparation of a medicament for treatment or prevention of a glucagon-related disease, disorder or condition.

10. The method or use according to claim 8 or 9, wherein the glucagon-related disease, disorder or condition is selected from diabetes, prediabetes syndrome, obesity, weight and/or appetite control, hyperlipidemia, and hyperglycemia.

11. The method or use according to claim 8 or 9, comprising administering to a subject a pharmaceutical composition according to any one of claims 1 to 6.

12. A method for preventing progression and/or treating a fatty liver disease with or without liver fibrosis, comprising administering a combination comprising a bitter receptor agonist comprising a denatonium salt and a GLP-1 receptor agonist.

13. The method of claim 12, wherein the dosage range of the denatonium salt for the method of treatment of NASH and related liver diseases in a human adult is from about 50 mg/day to about 3000 mg/day or from about 100 mg/day to about 2000 mg/day.

14. The method of claim 12, wherein the dosage range of the denatonium salt for the method of treatment of NASH and related liver diseases in a human adult is from about 0.5 mg/kg BID to about 30 mg/kg BID or from about 1 mg/kg BID to about 20 mg/kg BID.

15. Use of a compound comprising a bitter receptor agonist comprising a denatonium salt, wherein the denatonium salt is selected from the group consisting of denatonium acetate (DA), denatonium citrate, denatonium maleate, denatonium saccharide, and denatonium tartrate, wherein the compounds are administered as a racemic mixture or as enantiomers, diastereoisomers, or pharmaceutically acceptable salts, for preparation of a medicament for treatment or prevention of progression of NAFLD, NASH, or ASH in combination with a GLP-1 receptor agonist.

16. The use of claim 15, wherein the dosage range of the denatonium salt for the use for treatment of NASH and related liver in a human adult is from about 50 mg/day to about 3000 mg/day or from about 100 mg/day to about 2000 mg/day.

17. The use of claim 15, wherein the dosage range of the denatonium salt for the method of treatment of NASH and related liver diseases in a human adult is from about 0.5 mg/kg BID to about 30 mg/kg BID or from about 1 mg/kg BID to about 20 mg/kg BID.

18. The use of any one of claim 9 or claims 15 to 17, wherein the daily dose of the denatonium salt is administered once per day, twice per day or three times per day.

19. The method or use of any one of claims 11 to 17, wherein the fatty liver disease is selected from NASH, ASH, NAFLD, HIV-associated steatohepatitis, and liver fibrosis.

20. The method or use of any one of claims 11 to 17, wherein the fatty liver disease does not include liver fibrosis.

21. The method, use or pharmaceutical composition of any one of claims 1 to 4 and 6 to 20, wherein the denatonium salt is denatonium citrate, denatonium tartrate, denatonium acetate, denatonium maleate, or denatonium saccharide.

22. The method, use, or pharmaceutical composition of any one of claims 1 to 5 and 7 to 21, wherein the gut signaling compound is a GLP-1 receptor agonist selected from semaglutide, glyburide, liraglutide, dulaglutide, and albiglutide, a glucagon, exenatide, or lixisenatide.

23. The method, use or pharmaceutical composition of claim 22, wherein the gut-signaling compound is semaglutide.

24. The method, use or pharmaceutical composition of claim 22, wherein the gut-signaling compound is sitagliptin.

25. The method, use or pharmaceutical composition of claim 22, wherein the gut-signaling compound is liraglutide.

26. The method, use or pharmaceutical composition of any of the claims 1 to 4 or claims 6 to 24, wherein the bitter receptor agonist is DA.
